(19) United States
(12) Patent Application Publication
(10) Pub. No.: US 2019/0326518 A1
(43) Pub. Date: Oct. 24, 2019

(54) ORGANIC OPTOELECTRONIC DEVICE AND DISPLAY DEVICE

(71) Applicant: SAMSUNG SDI CO., LTD., Yongin-si, Gyeonggi-do (KR)

(72) Inventors: Jinhyun LUI, Suwon-si, Gyeonggi-do (KR); Ho Kuk JUNG, Suwon-si, Gyeonggi-do (KR); Dong-Yeong KIM, Suwon-si, Gyeonggi-do (KR); Kipo JANG, Suwon-si, Gyeonggi-do (KR); Dal-Ho HUH, Suwon-si, Gyeonggi-do (KR); Eun Sun YU, Suwon-si, Gyeonggi-do (KR); Sung-Hyun JUNG, Suwon-si, Gyeonggi-do (KR)

(21) Appl. No.: 16/349,719

(22) PCT Filed: Sep. 15, 2017
(86) PCT No.: PCT/KR2017/010143

§ 371 Date: May 14, 2019

(30) Foreign Application Priority Data
Nov. 24, 2016 (KR) 10-2016-0157490

(51) Int. Cl. H01L 51/00; C07D 405/14; C09K 11/06; C07D 409/14; C07D 209/86

(52) U.S. Cl. CPC H01L 51/0067; C07D 405/14; C09K 11/06; H01L 51/0073; H01L 51/5016; H01L 51/0074; C07D 209/86; H01L 51/0072; C07D 409/14

(57) ABSTRACT

Disclosed is an organic optoelectronic device including a cathode and an anode facing each other; a light emitting layer between the cathode and the anode; and an electron transport layer between the cathode and the light emitting layer, wherein the light emitting layer includes at least one of a first compound for an organic optoelectronic device represented by Chemical Formula 1 and at least one of a second compound for an organic optoelectronic device represented by Chemical Formula 2, and the electron transport layer includes at least one of a third compound for an organic optoelectronic device represented by Chemical Formula 3.

Details of Chemical Formulae 1 to 3 are the same as described in the specification.

### TECHNICAL FIELD

An organic optoelectronic device and a display device are disclosed.

### BACKGROUND ART

An organic optoelectronic device is a device that converts electrical energy into photoenergy, and vice versa.

An organic optoelectronic device may be classified as follows in accordance with its driving principles. One is a photoelectric device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectronic device may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode is a device converting electrical energy into light by applying current to an organic light emitting material, and has a structure in which an organic layer is disposed between an anode and a cathode. Herein, the organic layer may include a light emitting layer and optionally an auxiliary layer, and the auxiliary layer may be, for example at least one layer selected from a hole injection layer, a hole transport layer, an electron blocking layer, an electron transport layer, an electron injection layer, and a hole blocking layer.

Performance of an organic light emitting diode may be affected by characteristics of the organic layer, and among them, may be mainly affected by characteristics of an organic material of the organic layer.

Particularly, development for an organic material being capable of increasing hole and electron mobility and simultaneously increasing electrochemical stability is needed so that the organic light emitting diode may be applied to a large-size flat panel display.

### DISCLOSURE

### Technical Problem

An embodiment provides an organic optoelectronic device capable of realizing high efficiency and long life-span characteristics.

Another embodiment provides a display device including the organic optoelectronic device.

### Technical Solution

According to an embodiment, an organic optoelectronic device includes a cathode and an anode facing each other; a light emitting layer between the cathode and the anode; and an electron transport layer between the cathode and the light emitting layer, wherein the light emitting layer includes at least one of a first compound for an organic optoelectronic device represented by Chemical Formula 1 and at least one of a second compound for an organic optoelectronic device represented by Chemical Formula 2, and the electron transport layer includes at least one of a third compound for an organic optoelectronic device represented by Chemical Formula 3.

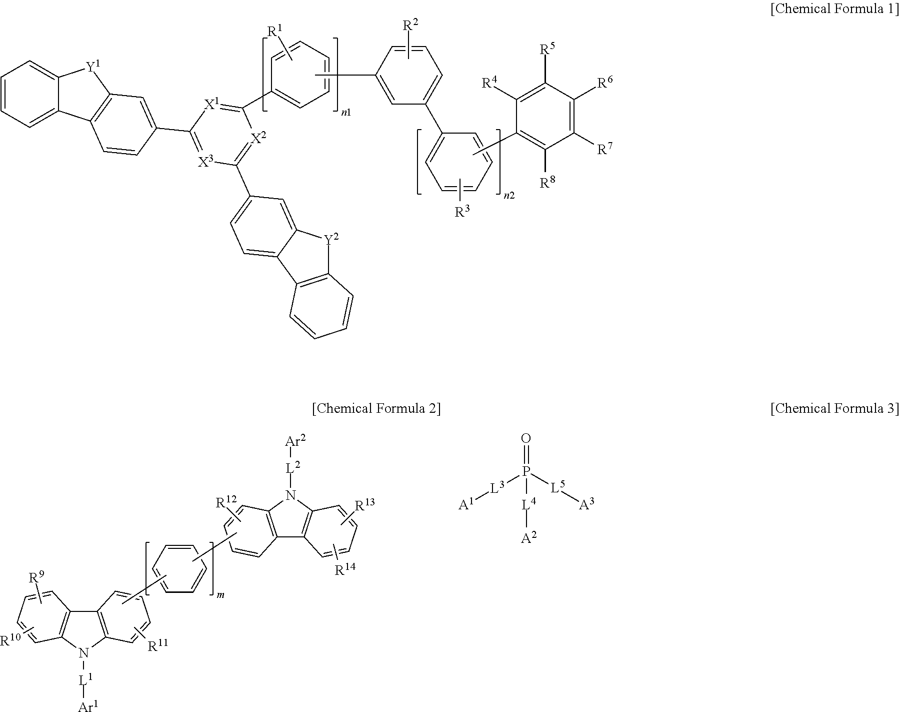

In Chemical Formula 1,

X¹ to X³ are independently N or CR^{a},

at least two of X¹ to X³ are N,

Y¹ and Y² are independently O or S,

n1 and n2 are independently an integer of 0 or 1, and

R^{a} and R¹ to R⁸ are independently hydrogen, deuterium, a cyano group, a nitro group, a substituted or unsubstituted C1 to C10 alkyl group, a substituted or unsubstituted C6 to C30 aryl group, a substituted or unsubstituted C2 to C30 heterocyclic group, or a combination thereof;

wherein, in Chemical Formula 2,

L¹ and L² are independently a single bond, a substituted or unsubstituted C6 to C30 arylene group, a substituted or unsubstituted C2 to C30 heteroarylene group, or a combination thereof,

Ar¹ and Ar² are independently a substituted or unsubstituted C6 to C30 aryl group, a substituted or unsubstituted carbazolyl group, a substituted or unsubstituted dibenzofuranyl group, a substituted or unsubstituted dibenzothiophenyl group, or a combination thereof,

R⁹ to R¹⁴ are independently hydrogen, deuterium, a substituted or unsubstituted C1 to C20 alkyl group, a substituted or unsubstituted C6 to C30 aryl group, a substituted or unsubstituted C2 to C30 heterocyclic group, or a combination thereof,

m is an integer of 0 to 2;

wherein, in Chemical Formula 3,

L³ to L⁵ are independently a single bond, a substituted or unsubstituted C6 to C30 arylene group, a substituted or unsubstituted C2 to C30 heteroarylene group, or a combination thereof,

A¹ to A³ are independently a substituted or unsubstituted C6 to C30 aryl group, a substituted or unsubstituted C2 to C30 heterocyclic group, or a combination thereof,

A¹ to A³ are independently present or adjacent groups are linked with each other to form at least one of a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring,

when A¹ to A³ are independently present, at least one of A¹ to A³ is a substituted or unsubstituted fused aryl group or a substituted or unsubstituted fused heterocyclic group, and

“substituted” of Chemical Formulae 1 to 3 refers to replacement of at least one hydrogen by deuterium, a C1 to C4 alkyl group, a C6 to C18 aryl group, or a C2 to C30 heteroaryl group.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIGS. 1 and 2 are cross-sectional views showing organic light emitting diodes according to embodiments.

### MODE FOR INVENTION

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, or a C2 to C30 heteroaryl group. In addition, in a specific example of the present invention, “substituted” refers to replacement of at least one hydrogen of a substituent or a compound by deuterium, a C1 to C20 alkyl group, a C6 to C30 aryl group, or a C2 to C30 heteroaryl group. In addition, in a specific example of the present invention, “substituted” refers to replacement of at least one hydrogen of a substituent or a compound by deuterium, a C1 to C5 alkyl group, a C6 to C18 aryl group, a dibenzofuranyl group, a dibenzothiophenyl group, or a carbazolyl group. In addition, in a specific example of the present invention, “substituted” refers to replacement of at least one hydrogen of a substituent or a compound by deuterium, a methyl group, an ethyl group, a propanyl group, a butyl group, a phenyl group, a biphenyl group, a terphenyl group, a naphthyl group, a triphenyl group, a fluorenyl group, a carbazolyl group, a dibenzofuranyl group, or a dibenzothiophenyl group.

In the present specification, when a definition is not otherwise provided, “hetero” refers to one including one to three heteroatoms selected from N, O, S, P, and Si, and remaining carbons in one functional group.

In the present specification, when a definition is not otherwise provided, “an alkyl group” refers to an aliphatic hydrocarbon group. The alkyl group may be “a saturated alkyl group” without any double bond or triple bond.

The alkyl group may be a C1 to C30 alkyl group. More specifically, the alkyl group may be a C1 to C20 alkyl group or a C1 to C10 alkyl group. For example, a C1 to C4 alkyl group may have one to four carbon atoms in the alkyl chain, and may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring. For example, it may be a fluorenyl group.

An aryl group may include a monocyclic, polycyclic or fused ring polycyclic (i.e., rings sharing adjacent pairs of carbon atoms) functional group.

In the present specification, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

Specific examples of the heterocyclic group may be a quinolinyl group, an isoquinolinyl group, a quinazolinyl group, a carbazolyl group, a dibenzofuranyl group, a dibenzothiophenyl group, and the like.

More specifically, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted benzoquinolinyl group, a substituted or unsubstituted benzoisoquinolinyl group, a substituted or unsubstituted benzoquinazolinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but are not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

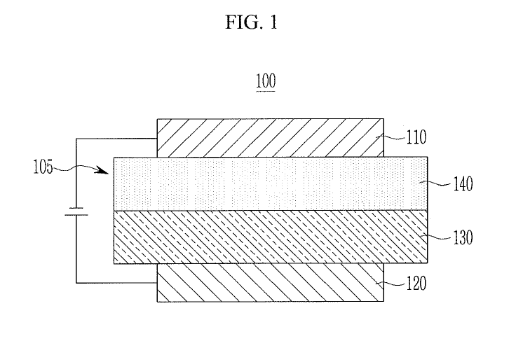

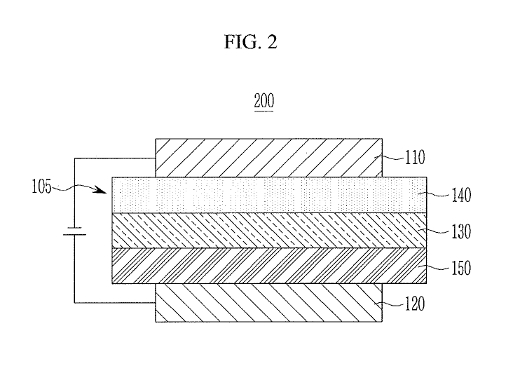

Hereinafter, an organic optoelectronic device according to an embodiment is described with reference to FIGS. 1 and 2.

An organic light emitting diode as one example of an organic optoelectronic device is described, but the present invention may be applied to other organic optoelectronic devices in the same way.

FIGS. 1 and 2 are schematic cross-sectional views of organic light emitting diodes.

Referring to FIG. 1, an organic light emitting diode according to an embodiment includes a cathode 110 and an anode 120 facing each other; and an organic layer 105 between the cathode 110 and the anode 120.

The organic layer 105 includes a light emitting layer 130 and an electron transport layer 140 between the cathode 110 and the light emitting layer 130.

According to an embodiment of the present invention, the light emitting layer may include at least one of a first compound for an organic optoelectronic device represented by Chemical Formula 1 and at least one of a second compound for an organic optoelectronic device represented by Chemical Formula 2, and the electron transport layer may include at least one of a third compound for an organic optoelectronic device represented by Chemical Formula 3.

In the organic layer, low driving and high efficiency characteristics may be maximized by including at least one of the first compound for an organic optoelectronic device represented by Chemical Formula 1 and at least one of the second compound for an organic optoelectronic device represented by Chemical Formula 2 in the light emitting layer, and simultaneously at least one of the third compound for an organic optoelectronic device represented by Chemical Formula 3 in the electron transport layer.

Specifically, the first compound for an organic optoelectronic device and the second compound for an organic optoelectronic device are used together in the light emitting layer and thus mobility and stability of charges are increased and luminous efficiency and life-span characteristics may be improved and the third compound for an organic optoelectronic device having a large dipole moment is simultaneously applied to the electron transport layer, and thus a driving voltage may be particularly lowered while maintaining a long life-span and high efficiency.

The light emitting layer 130 is an organic layer emitting light and includes a host and a dopant when a doping system is adopted. Herein, the host mainly promotes a recombination of electrons and confines excitons in a light emitting layer, while the dopant efficiently emits light from the excitons obtained from the recombination.

The light emitting layer 130 includes at least two kinds of hosts and dopants, and the hosts include a first compound for an organic optoelectronic device having relatively strong electron characteristics and a second compound for an organic optoelectronic device having strong hole characteristics.

The first compound for an organic optoelectronic device is represented by Chemical Formula 1.

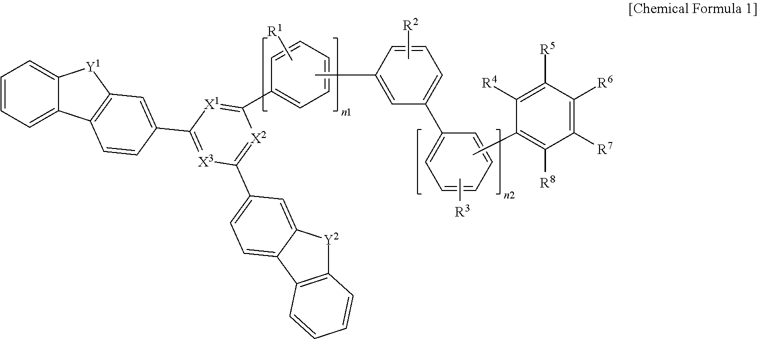

In Chemical Formula 1,

X¹ to X³ are independently N or CR^{a},

at least two of X¹ to X³ are N,

Y¹ and Y² are independently O or S,

n1 and n2 are independently an integer of 0 or 1,

R^{a} and R¹ to R⁸ are independently hydrogen, deuterium, a cyano group, a nitro group, a substituted or unsubstituted C1 to C10 alkyl group, a substituted or unsubstituted C6 to C30 aryl group, a substituted or unsubstituted C2 to C30 heterocyclic group, or a combination thereof, and

the “substituted” refers to replacement of at least one hydrogen by deuterium, a C1 to C20 alkyl group, a C6 to C30 aryl group, or a C2 to C30 heteroaryl group.

In one example of the present invention, the “substituted” in Chemical Formula 1 may refer to replacement of at least one hydrogen by deuterium, a C1 to C4 alkyl group, a C6 to C20 aryl group, or a C2 to C20 heteroaryl group, and specifically the “substituted” may refer to replacement of at least one hydrogen by deuterium, a C1 to C4 alkyl group, a phenyl group, a biphenyl group, a terphenyl group, a dibenzofuranyl group, or a dibenzothiophenyl group.

The first compound for an organic optoelectronic device includes an ET core including an N-containing 6-membered ring that is directly linked with at least two dibenzofuran or dibenzothiophene at the position No. 3 without a linking group, and thereby a LUMO energy band is effectively expanded and planarity of molecular structure is increased, the first compound has a structure easily to accept electrons when an electric field is applied, and thus an organic optoelectronic device including the compound for an organic optoelectronic device has a lowered driving voltage. In addition, such an expansion of LUMO and fusion of rings increase stability for electrons of the ET core and improves life-span effectively.

In addition, interactions with neighboring molecules may be suppressed and crystallization may be decreased due to steric hindrance characteristics by including at least one meta-bound arylene, and thus an organic optoelectronic device including the compound for an organic optoelectronic device may improve efficiency and life-span characteristics.

Furthermore, when a kinked moiety such as a meta-bound arylene is included, a compound may have an increased glass transition temperature (Tg) and stability and may suppress degradation during application of a device.

In addition, in an example embodiment of the present invention, the number of the phenyl groups linked with a central 6-membered ring of Chemical Formula 1 of Chemical Formula 1 may be at least three, which may exhibit more improved effects. Herein, at least one of three phenyl groups may be desirably meta-bound and the three phenyl groups may be linear or branched.

In an example embodiment of the present invention, an ET core consisting of X¹ to X³ may be pyrimidine or triazine, and may be for example represented by Chemical Formula 1-I, Chemical Formula 1-II, or Chemical Formula 1-III. More specifically, it may be represented by Chemical Formula 1-I or Chemical Formula 1-II.

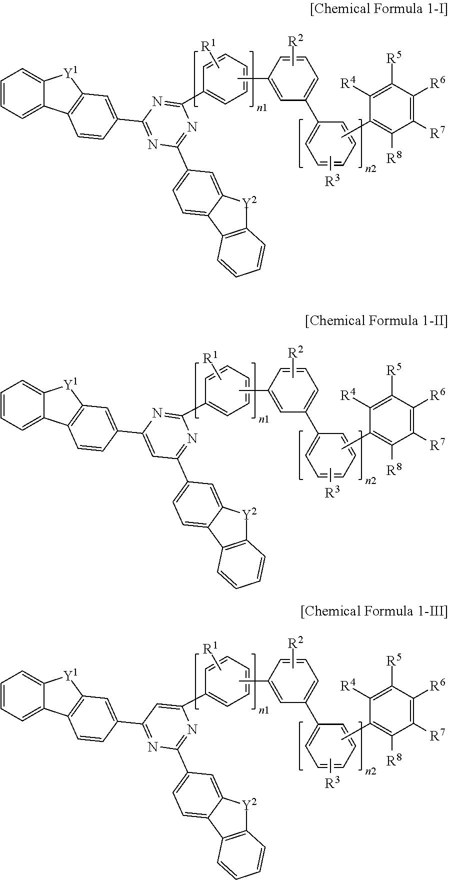

In Chemical Formula 1-I, Chemical Formula 1-I, and Chemical Formula 1-I, Y and Y², n1 and n2 and R¹ to R⁸ are the same as described above.

In an example embodiment of the present invention, R¹ to R⁸ may be independently hydrogen or a substituted or unsubstituted C6 to C30 aryl group, specifically hydrogen, a substituted or unsubstituted phenyl group, a substituted or unsubstituted biphenyl group, a substituted or unsubstituted naphthyl group, a substituted or unsubstituted p-terphenyl group, a substituted or unsubstituted m-terphenyl group, a substituted or unsubstituted o-terphenyl group, a substituted or unsubstituted anthracenyl group, a substituted or unsubstituted phenanthrenyl group, a substituted or unsubstituted triphenylenyl group, or a substituted or unsubstituted fluorenyl group, and more specifically hydrogen, a phenyl group, a biphenyl group, a terphenyl group, or a naphthyl group.

For example, R¹ to R³ may independently be hydrogen, deuterium, a phenyl group, a biphenyl group, or a naphthyl group.

In addition, in one example of the present invention, one of R⁴ to R⁸ may be deuterium, a phenyl group, a biphenyl group, or a terphenyl group and the rest may be hydrogen.

In addition, in one example of the present invention, one of R⁵ and R⁷ or one of R⁵ and R⁷ may be deuterium, hydrogen, a phenyl group, a biphenyl group, or a terphenyl group and all R⁴, R⁶, and R⁸ may be hydrogen.

For example, R¹ may be hydrogen or a phenyl group, all R² and R³ may be hydrogen, and all R⁴ to R⁸ may be hydrogen or one of R⁴ to R⁸ may be a phenyl group, a biphenyl group, or a terphenyl group and the rest may be hydrogen.

In one example of the present invention, R¹ may be a phenyl group.

Chemical Formula 1 may be for example represented by Chemical Formula 1A, Chemical Formula 1B, or Chemical Formula 1C.

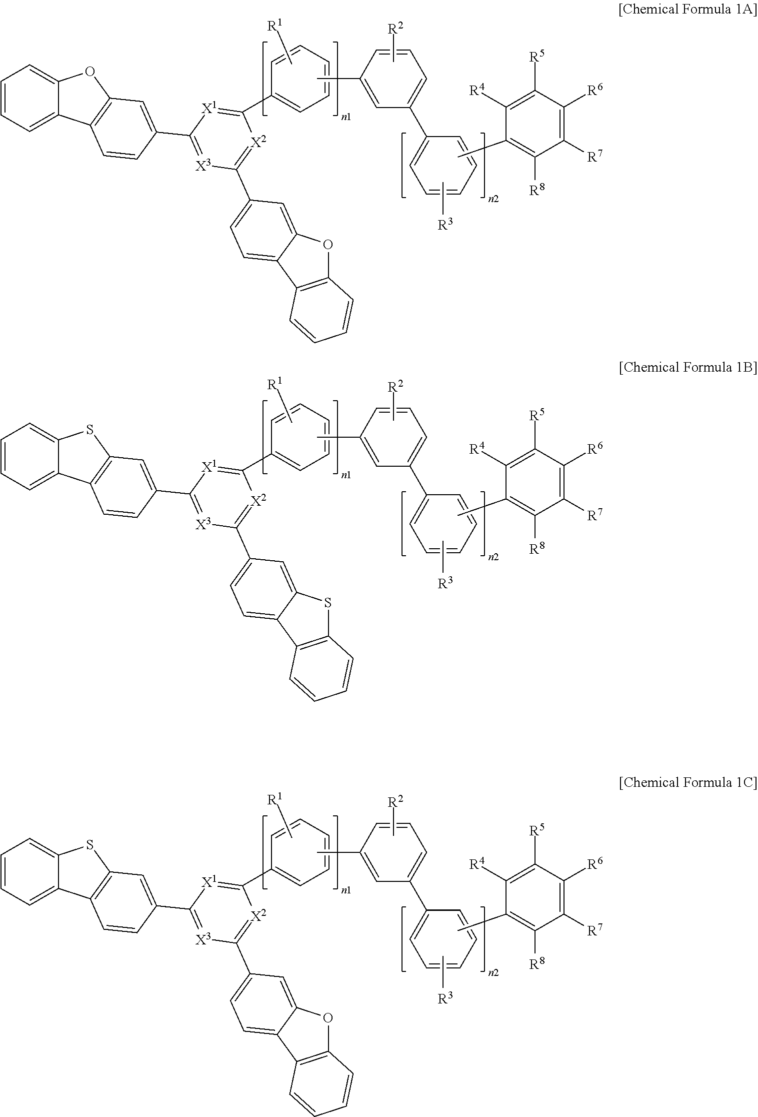

In Chemical Formula 1A, Chemical Formula 1B, and Chemical Formula 1C, n1 and n2, and R¹ to R⁸ are the same as above, and

X¹ to X³ may independently be N or CH and at least two of X¹ to X³ may be N.

As in Chemical Formulae 1A to 1C, when the dibenzofuranyl group and/or the dibenzothiophenyl group is directly linked with the N-containing 6-membered ring at the position No. 3 without a linking group, a LUMO phore may be positioned in one plane to maximize the expansion effect, and optimal effects in terms of low driving and an increase of a life-span may be obtained. When the dibenzofuran and/or dibenzothiophene is linked with the N-containing 6-membered ring at other positions except No. 3 or an arylene linker is included between the N-containing 6-membered ring and the dibenzofuran and/or dibenzothiophene, a driving decrease through the LUMO expansion and an increase of stability through fusion of rings may be reduced.

In an example embodiment of the present invention, Chemical Formula 1 may be represented by Chemical Formula 1A, or Chemical Formula 1B, and may be for example represented by Chemical Formula 1A.

In an example embodiment of the present invention, the n1 and n2 may be 0, n1=1 and n2=0; or n1=0 and n2=1, Chemical Formula 1 has a structure including a meta-bound arylene, and may be for example represented by Chemical Formula 1-1 or Chemical Formula 1-2, and may be more specifically represented by Chemical Formula 1-1.

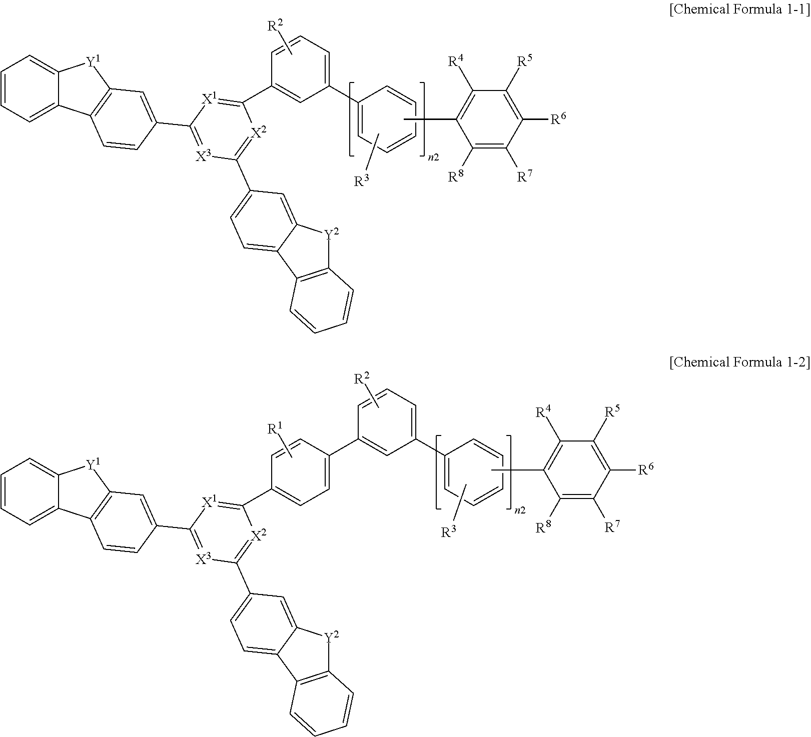

In Chemical Formulae 1-1 to 1-2, X¹ to X³, Y¹ and Y², n2 and R¹ to R⁸ are the same as described above.

Particularly, R² of Chemical Formulae 1-1 and 1-2 may be a substituted or unsubstituted C1 to C10 alkyl group, a substituted or unsubstituted C6 to C30 aryl group, or a substituted or unsubstituted C2 to C30 heterocyclic group, and more specifically R² is bound at a meta position wherein Chemical Formula 1 may be represented by Chemical Formula 1-1a or Chemical Formula 1-2a. Herein, R²-bound phenylene may include a kinked terphenyl group.

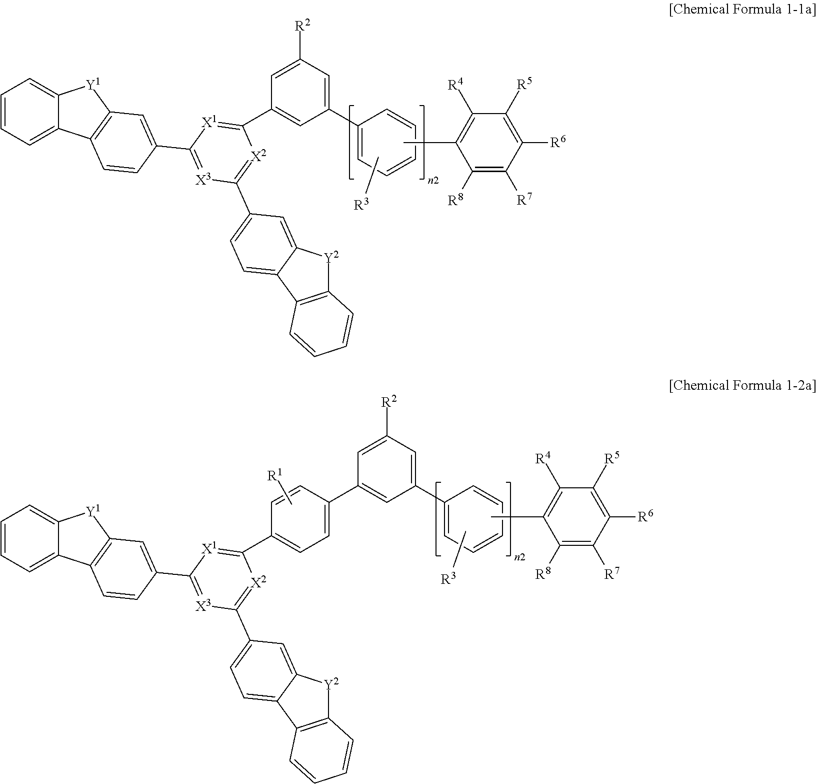

In an example embodiment of the present invention, R² may be a substituted or unsubstituted C1 to C4 alkyl group or a substituted or unsubstituted C6 to C30 aryl group, and may be for example a phenyl group, a biphenyl group, a terphenyl group, or a naphthyl group and more specifically a substituted or unsubstituted phenyl group.

That is, when the substituted or unsubstituted C6 to C30 aryl group of R² includes a substituted the kinked terphenyl group, a glass transition temperature (Tg) may be increased very effectively, a compound having a low molecular weight and a high glass transition temperature (Tg) may be designed, and thereby thermal characteristics may be improved and stability may be ensured.

The glass transition temperature (Tg) may be related with thermal stability of a compound and a device including the compound. That is, when a compound for an organic optoelectronic device having a high glass transition temperature (Tg) is applied to an organic light emitting diode in a form of a thin film, degradation by the temperature may be suppressed in a subsequent process, for example an encapsulation process after depositing the compound for an organic optoelectronic device, life-span characteristics of the organic compound and a device may be ensured.

On the other hand, in Chemical Formulae 1-1 and 1-2, a linking group represented by

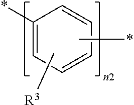

may be meta-bound or para-bound.

The compound for an organic optoelectronic device represented by Chemical Formula 1 may be for example selected from compounds of Group 1, but is not limited thereto.

### [Group 1]

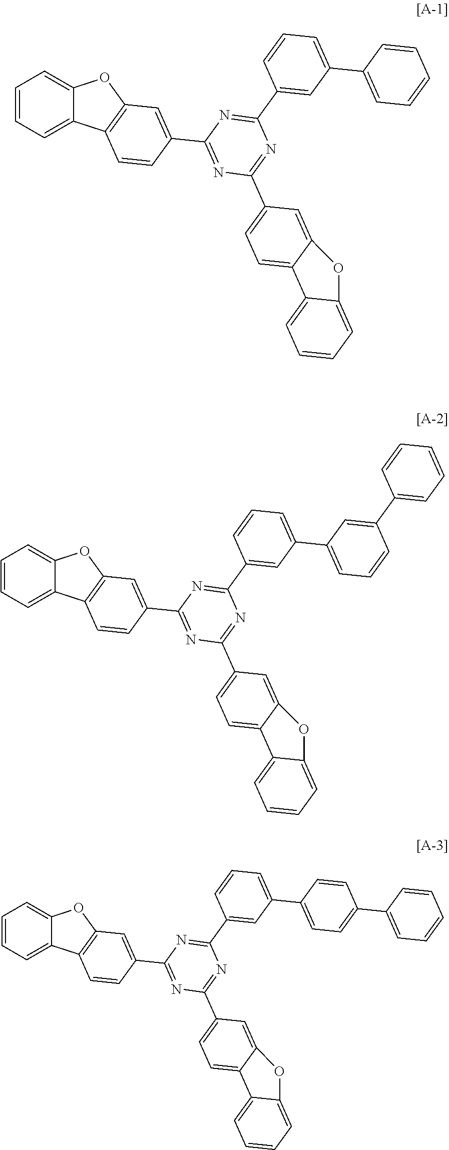 [SMILES: c1ccc(-c2cccc(-c3nc(-c4ccc5c(c4)oc4ccccc45)nc(-c4ccc5c(c4)oc4ccccc45)n3)c2)cc1 ; c1ccc(-c2cccc(-c3cccc(-c4nc(-c5ccc6c(c5)oc5ccccc56)nc(-c5ccc6c(c5)oc5ccccc56)n4)c3)c2)cc1 ; c1ccc(-c2ccc(-c3cccc(-c4nc(-c5ccc6c(c5)oc5ccccc56)nc(-c5ccc6c(c5)oc5ccccc56)n4)c3)cc2)cc1]

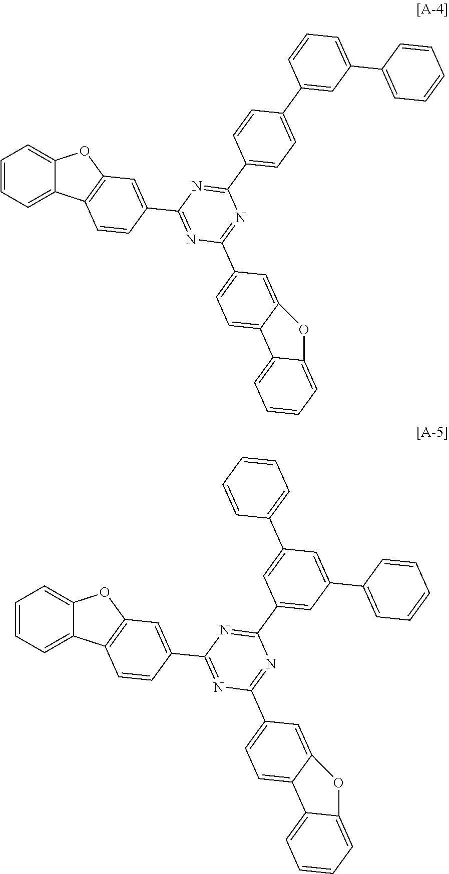 [SMILES: c1ccc(-c2cccc(-c3ccc(-c4nc(-c5ccc6c(c5)oc5ccccc56)nc(-c5ccc6c(c5)oc5ccccc56)n4)cc3)c2)cc1 ; c1ccc(-c2cc(-c3ccccc3)cc(-c3nc(-c4ccc5c(c4)oc4ccccc45)nc(-c4ccc5c(c4)oc4ccccc45)n3)c2)cc1]

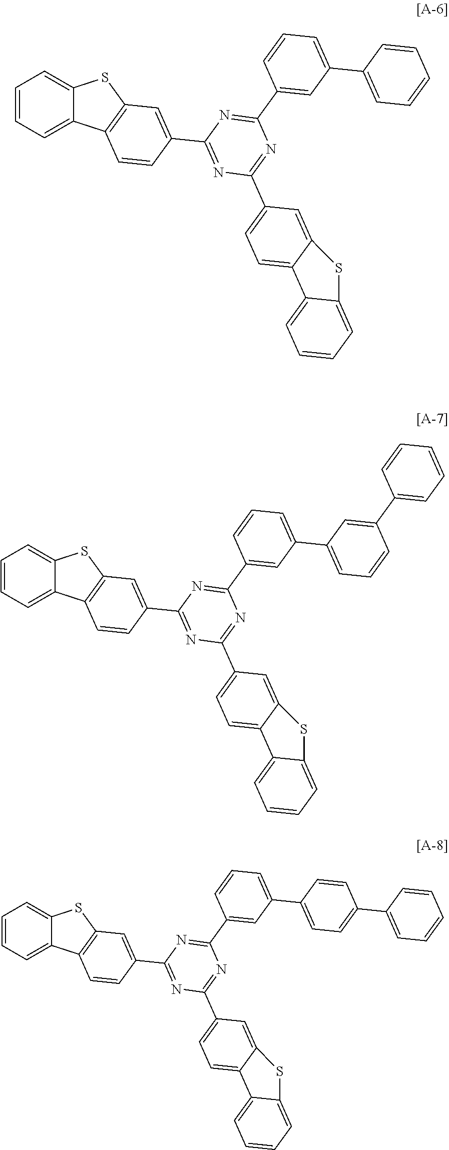 [SMILES: c1ccc(-c2cccc(-c3nc(-c4ccc5c(c4)sc4ccccc45)nc(-c4ccc5c(c4)sc4ccccc45)n3)c2)cc1 ; c1ccc(-c2cccc(-c3cccc(-c4nc(-c5ccc6c(c5)sc5ccccc56)nc(-c5ccc6c(c5)sc5ccccc56)n4)c3)c2)cc1 ; c1ccc(-c2ccc(-c3cccc(-c4nc(-c5ccc6c(c5)sc5ccccc56)nc(-c5ccc6c(c5)sc5ccccc56)n4)c3)cc2)cc1]

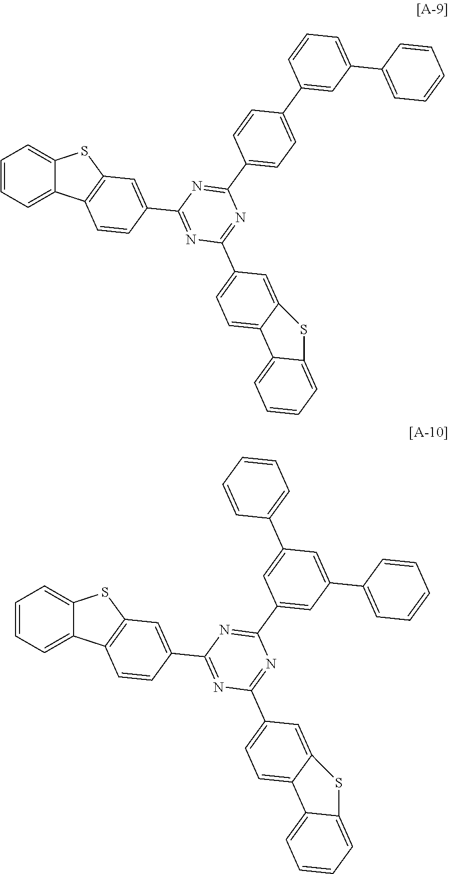 [SMILES: c1ccc(-c2cccc(-c3ccc(-c4nc(-c5ccc6c(c5)sc5ccccc56)nc(-c5ccc6c(c5)sc5ccccc56)n4)cc3)c2)cc1 ; c1ccc(-c2cc(-c3ccccc3)cc(-c3nc(-c4ccc5c(c4)sc4ccccc45)nc(-c4ccc5c(c4)sc4ccccc45)n3)c2)cc1]

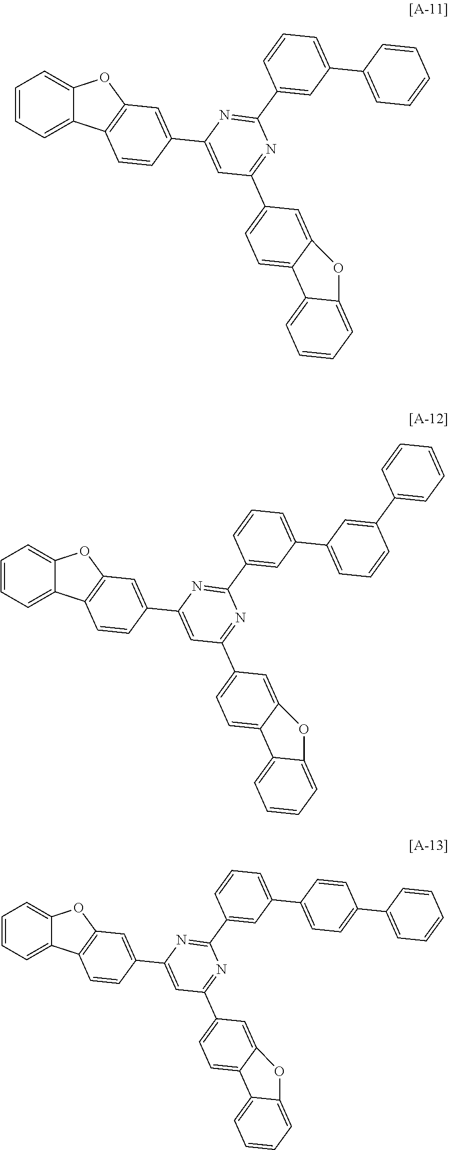 [SMILES: c1ccc(-c2cccc(-c3nc(-c4ccc5c(c4)oc4ccccc45)cc(-c4ccc5c(c4)oc4ccccc45)n3)c2)cc1 ; c1ccc(-c2cccc(-c3cccc(-c4nc(-c5ccc6c(c5)oc5ccccc56)cc(-c5ccc6c(c5)oc5ccccc56)n4)c3)c2)cc1 ; c1ccc(-c2ccc(-c3cccc(-c4nc(-c5ccc6c(c5)oc5ccccc56)cc(-c5ccc6c(c5)oc5ccccc56)n4)c3)cc2)cc1]

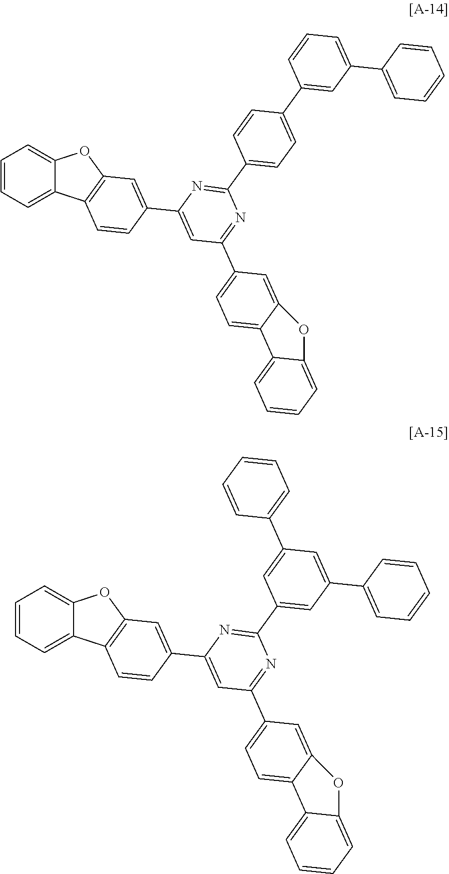 [SMILES: c1ccc(-c2cccc(-c3ccc(-c4nc(-c5ccc6c(c5)oc5ccccc56)cc(-c5ccc6c(c5)oc5ccccc56)n4)cc3)c2)cc1 ; c1ccc(-c2cc(-c3ccccc3)cc(-c3nc(-c4ccc5c(c4)oc4ccccc45)cc(-c4ccc5c(c4)oc4ccccc45)n3)c2)cc1]

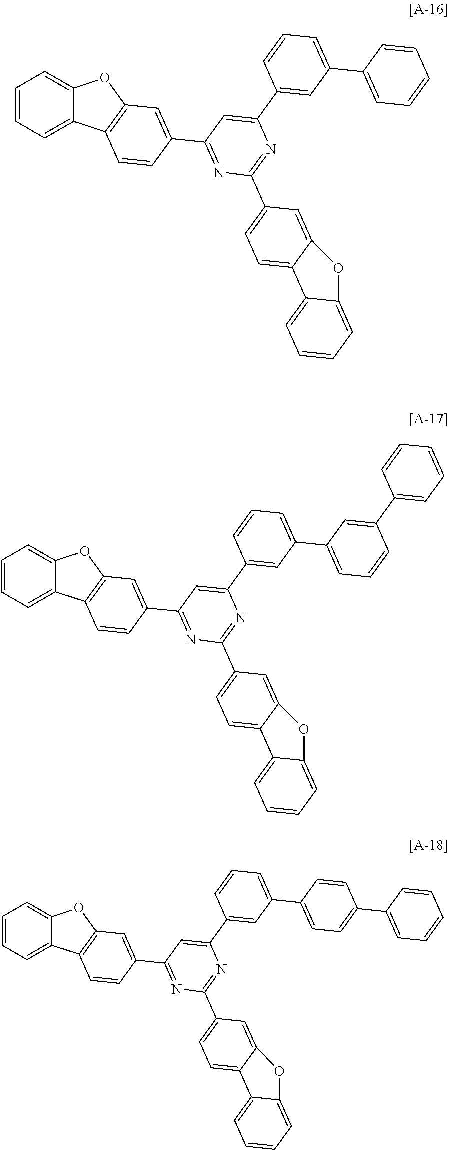 [SMILES: c1ccc(-c2cccc(-c3cc(-c4ccc5c(c4)oc4ccccc45)nc(-c4ccc5c(c4)oc4ccccc45)n3)c2)cc1 ; c1ccc(-c2cccc(-c3cccc(-c4cc(-c5ccc6c(c5)oc5ccccc56)nc(-c5ccc6c(c5)oc5ccccc56)n4)c3)c2)cc1 ; c1ccc(-c2ccc(-c3cccc(-c4cc(-c5ccc6c(c5)oc5ccccc56)nc(-c5ccc6c(c5)oc5ccccc56)n4)c3)cc2)cc1]

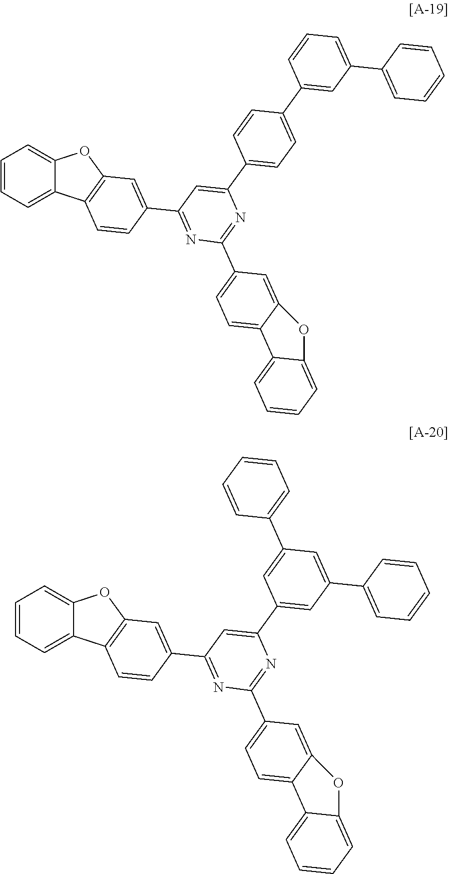 [SMILES: c1ccc(-c2cccc(-c3ccc(-c4cc(-c5ccc6c(c5)oc5ccccc56)nc(-c5ccc6c(c5)oc5ccccc56)n4)cc3)c2)cc1 ; c1ccc(-c2cc(-c3ccccc3)cc(-c3cc(-c4ccc5c(c4)oc4ccccc45)nc(-c4ccc5c(c4)oc4ccccc45)n3)c2)cc1]

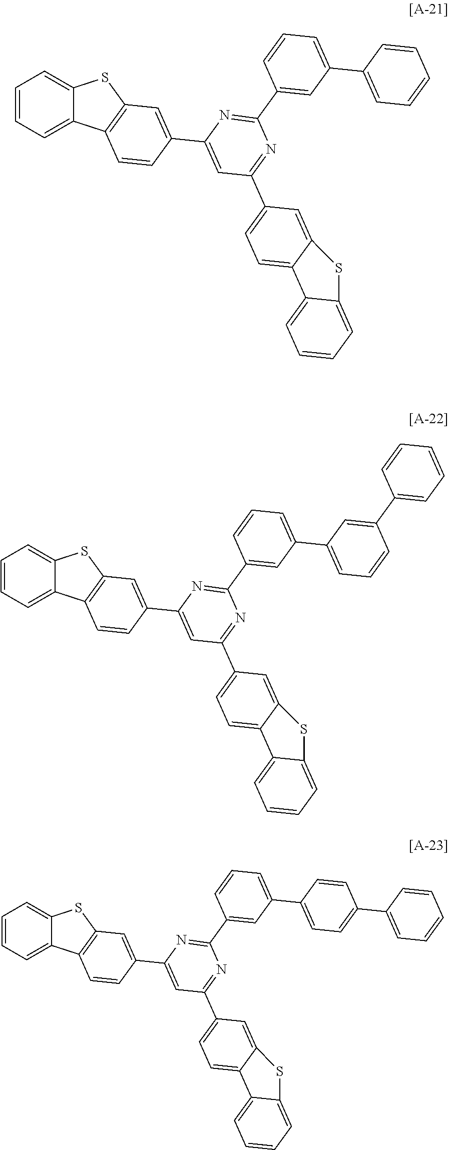 [SMILES: c1ccc(-c2cccc(-c3nc(-c4ccc5c(c4)sc4ccccc45)cc(-c4ccc5c(c4)sc4ccccc45)n3)c2)cc1 ; c1ccc(-c2cccc(-c3cccc(-c4nc(-c5ccc6c(c5)sc5ccccc56)cc(-c5ccc6c(c5)sc5ccccc56)n4)c3)c2)cc1 ; c1ccc(-c2ccc(-c3cccc(-c4nc(-c5ccc6c(c5)sc5ccccc56)cc(-c5ccc6c(c5)sc5ccccc56)n4)c3)cc2)cc1]

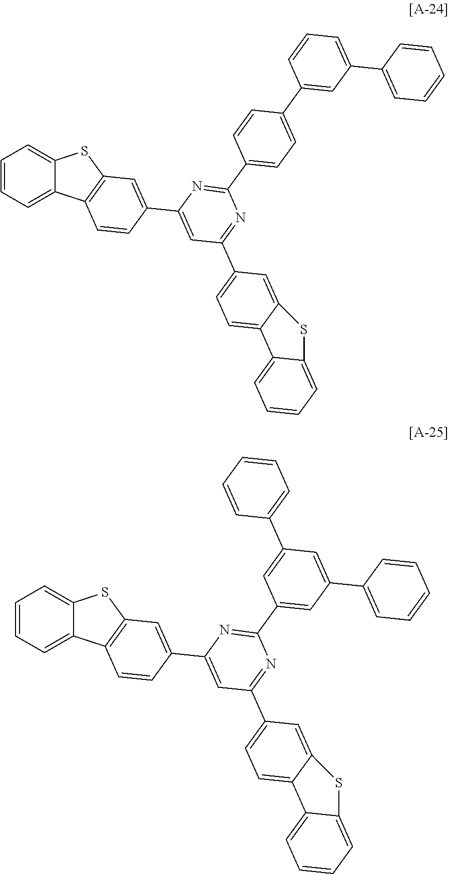 [SMILES: c1ccc(-c2cccc(-c3ccc(-c4nc(-c5ccc6c(c5)sc5ccccc56)cc(-c5ccc6c(c5)sc5ccccc56)n4)cc3)c2)cc1 ; c1ccc(-c2cc(-c3ccccc3)cc(-c3nc(-c4ccc5c(c4)sc4ccccc45)cc(-c4ccc5c(c4)sc4ccccc45)n3)c2)cc1]

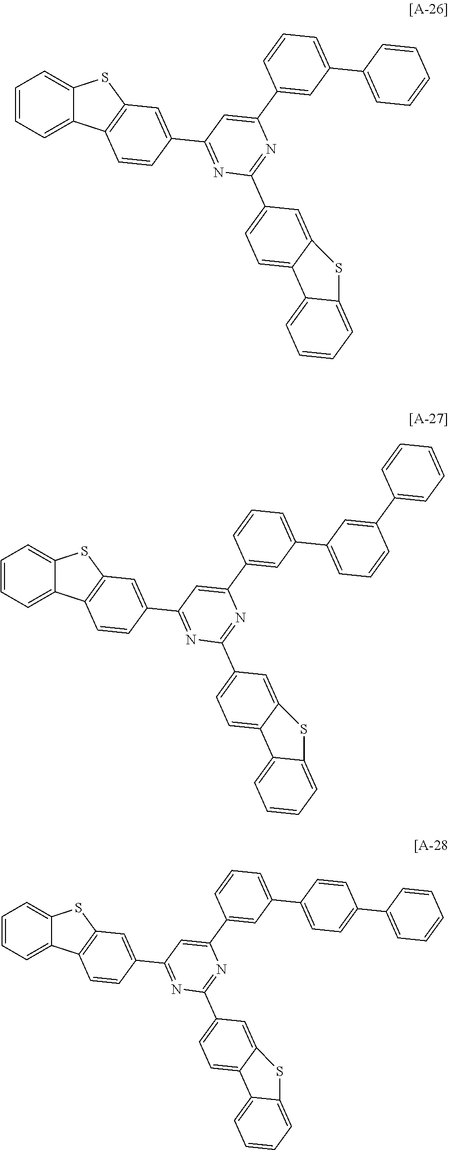 [SMILES: c1ccc(-c2cccc(-c3cc(-c4ccc5c(c4)sc4ccccc45)nc(-c4ccc5c(c4)sc4ccccc45)n3)c2)cc1 ; c1ccc(-c2cccc(-c3cccc(-c4cc(-c5ccc6c(c5)sc5ccccc56)nc(-c5ccc6c(c5)sc5ccccc56)n4)c3)c2)cc1 ; c1ccc(-c2ccc(-c3cccc(-c4cc(-c5ccc6c(c5)sc5ccccc56)nc(-c5ccc6c(c5)sc5ccccc56)n4)c3)cc2)cc1]

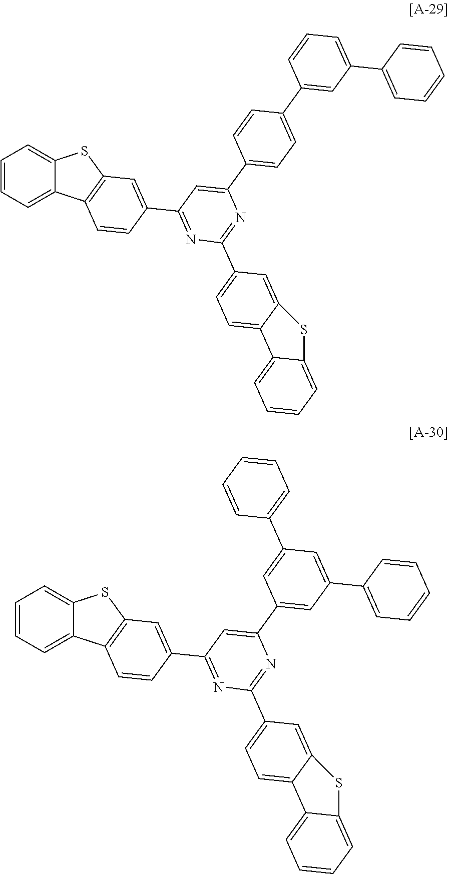 [SMILES: c1ccc(-c2cccc(-c3ccc(-c4cc(-c5ccc6c(c5)sc5ccccc56)nc(-c5ccc6c(c5)sc5ccccc56)n4)cc3)c2)cc1 ; c1ccc(-c2cc(-c3ccccc3)cc(-c3cc(-c4ccc5c(c4)sc4ccccc45)nc(-c4ccc5c(c4)sc4ccccc45)n3)c2)cc1]

The second compound for an organic optoelectronic device may be represented by Chemical Formula 2.

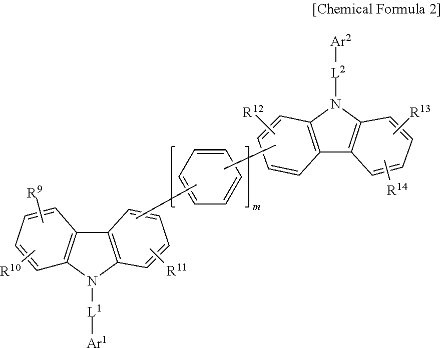

In Chemical Formula 2. L¹ and L² are independently a single bond, a substituted or unsubstituted C6 to C30 arylene group, a substituted or unsubstituted C2 to C30 heteroarylene group, or a combination thereof.

Ar¹ and Ar² are independently a substituted or unsubstituted C6 to C30 aryl group, a substituted or unsubstituted carbazolyl group, a substituted or unsubstituted dibenzofuranyl group, a substituted or unsubstituted dibenzothiophenyl group, or a combination thereof,

R⁹ to R¹⁴ are independently hydrogen, deuterium, a substituted or unsubstituted C1 to C20 alkyl group, a substituted or unsubstituted C6 to C30 aryl group, a substituted or unsubstituted C2 to C30 heterocyclic group, or a combination thereof, and

m is an integer of 0 to 2;

wherein the “substituted” refers to replacement of at least one hydrogen by deuterium, a C1 to C4 alkyl group, a C6 to C18 aryl group, or a C2 to C30 heteroaryl group.

In one example of the present invention, the “substituted” of Chemical Formula 2 may refer to replacement of at least one hydrogen by deuterium, a C1 to C4 alkyl group, a C6 to C20 aryl group, or a C2 to C20 heteroaryl group, and specifically the “substituted” may refer to replacement of at least one hydrogen by deuterium, a C1 to C4 alkyl group, a phenyl group, a biphenyl group, a terphenyl group, a fluorenyl group, a triphenylene group, a carbazolyl group, a dibenzofuranyl group, or a dibenzothiophenyl group.

In an example embodiment of the present invention, L¹ and L² of Chemical Formula 2 may independently be a single bond, or a substituted or unsubstituted C6 to C18 arylene group.

In an example embodiment of the present invention, Ar¹ and Ar² of Chemical Formula 2 may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted dibenzothiophenyl group, a substituted or unsubstituted dibenzofuranyl group, a substituted or unsubstituted carbazolyl group, a substituted or unsubstituted fluorenyl group, or a combination thereof.

In an example embodiment of the present invention, R⁹ to R¹⁴ of Chemical Formula 2 may independently be hydrogen, deuterium, or a substituted or unsubstituted C6 to C12 aryl group.

In an example embodiment of the present invention, m of Chemical Formula 2 may be 0 or 1.

In a specific example embodiment of the present invention, Chemical Formula 2 may be one of structures of Group I and *-L¹-Ar¹ and *-L²-Ar² may be one of substituents of Group II.

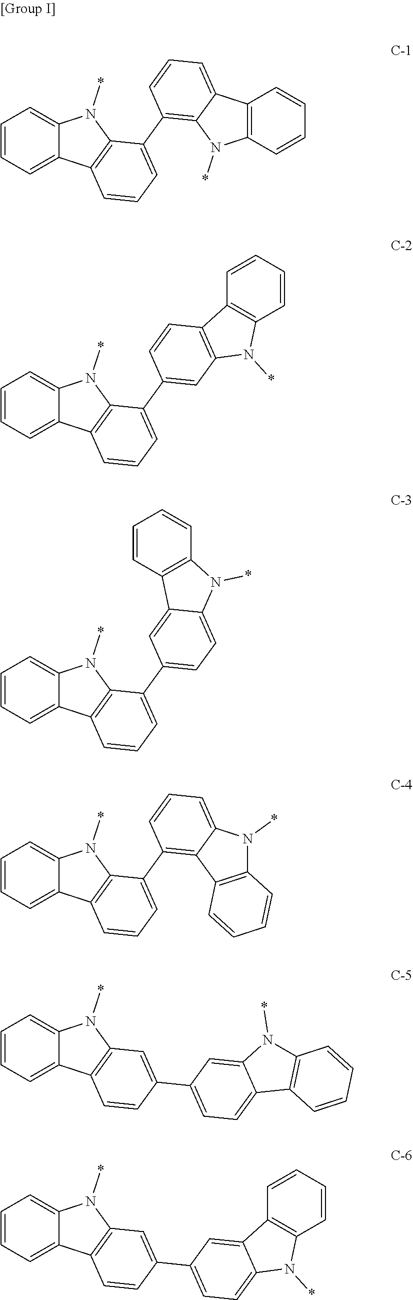 [SMILES: *n1c2ccccc2c2cccc(-c3cccc4c5ccccc5n(*)c34)c21 ; *n1c2ccccc2c2ccc(-c3cccc4c5ccccc5n(*)c34)cc21 ; *n1c2ccccc2c2cc(-c3cccc4c5ccccc5n(*)c34)ccc21 ; *n1c2ccccc2c2c(-c3cccc4c5ccccc5n(*)c34)cccc21 ; *n1c2ccccc2c2ccc(-c3ccc4c5ccccc5n(*)c4c3)cc21 ; *n1c2ccccc2c2cc(-c3ccc4c5ccccc5n(*)c4c3)ccc21]

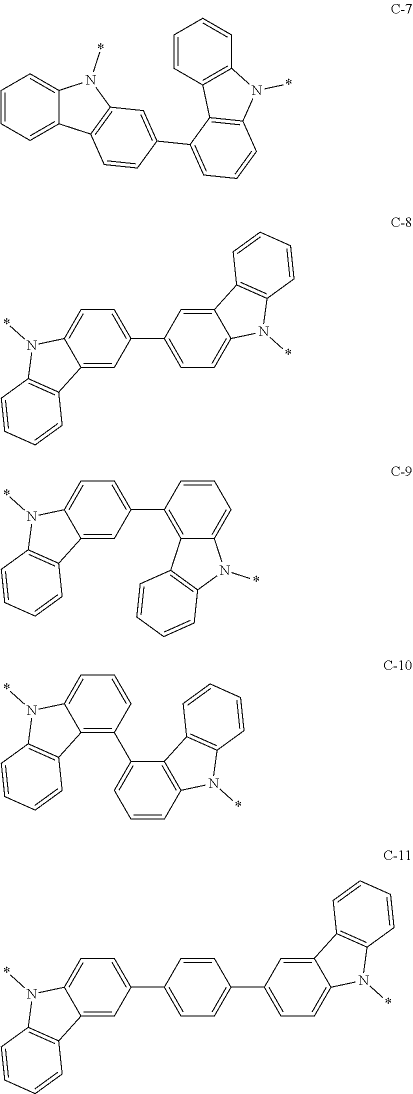 [SMILES: *n1c2ccccc2c2ccc(-c3cccc4c3c3ccccc3n4*)cc21 ; *n1c2ccccc2c2cc(-c3ccc4c(c3)c3ccccc3n4*)ccc21 ; *n1c2ccccc2c2cc(-c3cccc4c3c3ccccc3n4*)ccc21 ; *n1c2ccccc2c2c(-c3cccc4c3c3ccccc3n4*)cccc21 ; *n1c2ccccc2c2cc(-c3ccc(-c4ccc5c(c4)c4ccccc4n5*)cc3)ccc21]

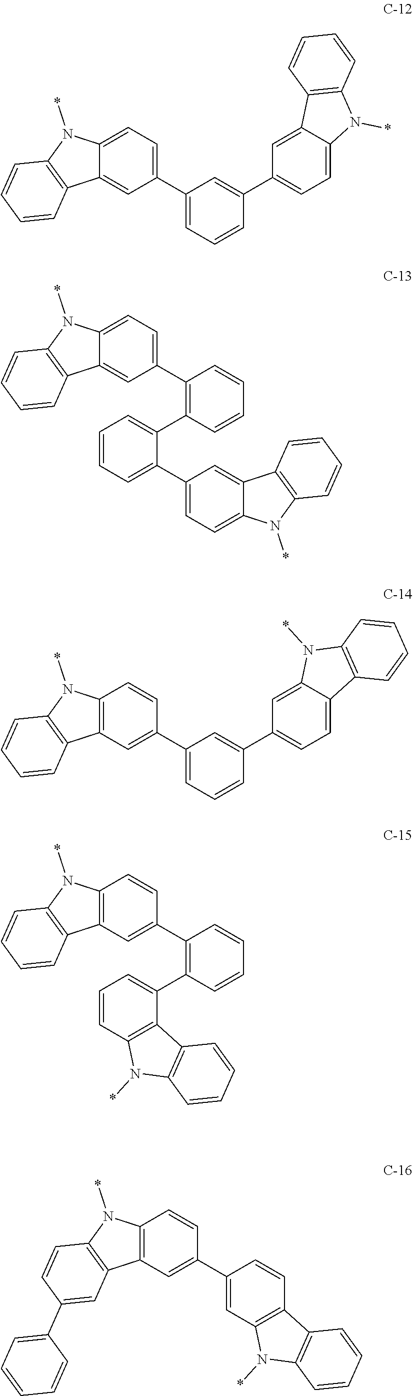 [SMILES: *n1c2ccccc2c2cc(-c3cccc(-c4ccc5c(c4)c4ccccc4n5*)c3)ccc21 ; *n1c2ccccc2c2cc(-c3ccccc3-c3ccccc3-c3ccc4c(c3)c3ccccc3n4*)ccc21 ; *n1c2ccccc2c2cc(-c3cccc(-c4ccc5c6ccccc6n(*)c5c4)c3)ccc21 ; *n1c2ccccc2c2cc(-c3ccccc3-c3cccc4c3c3ccccc3n4*)ccc21 ; *n1c2ccc(-c3ccccc3)cc2c2cc(-c3ccc4c5ccccc5n(*)c4c3)ccc21]

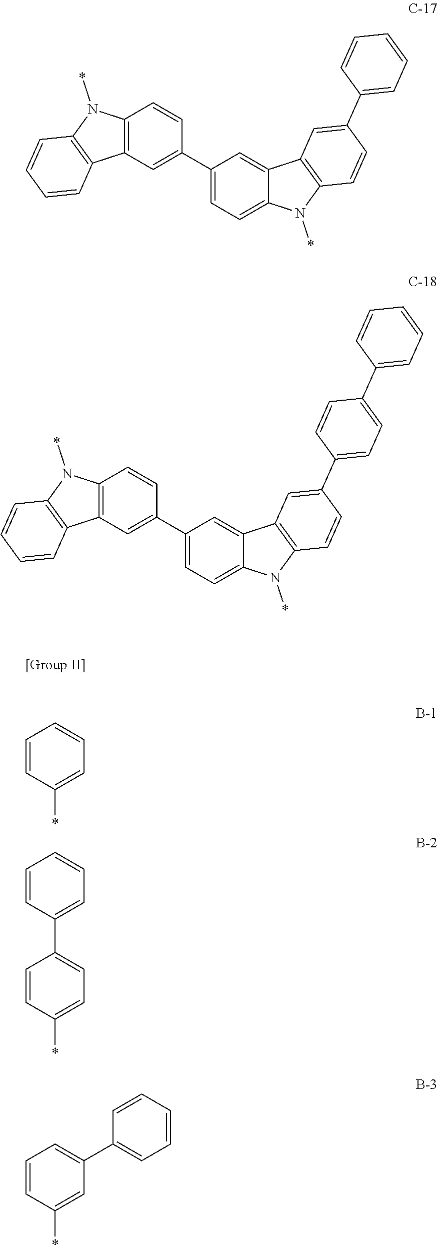

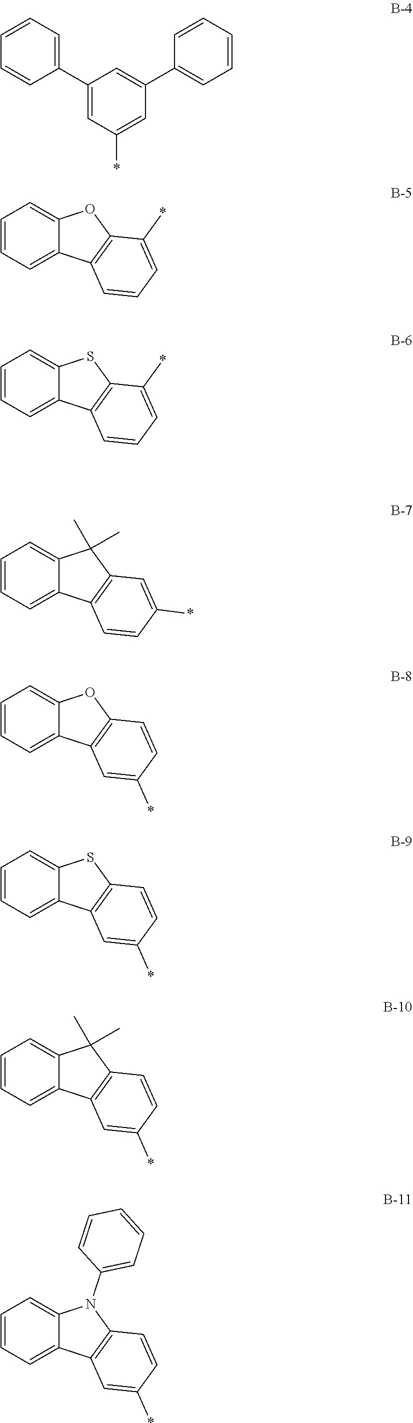

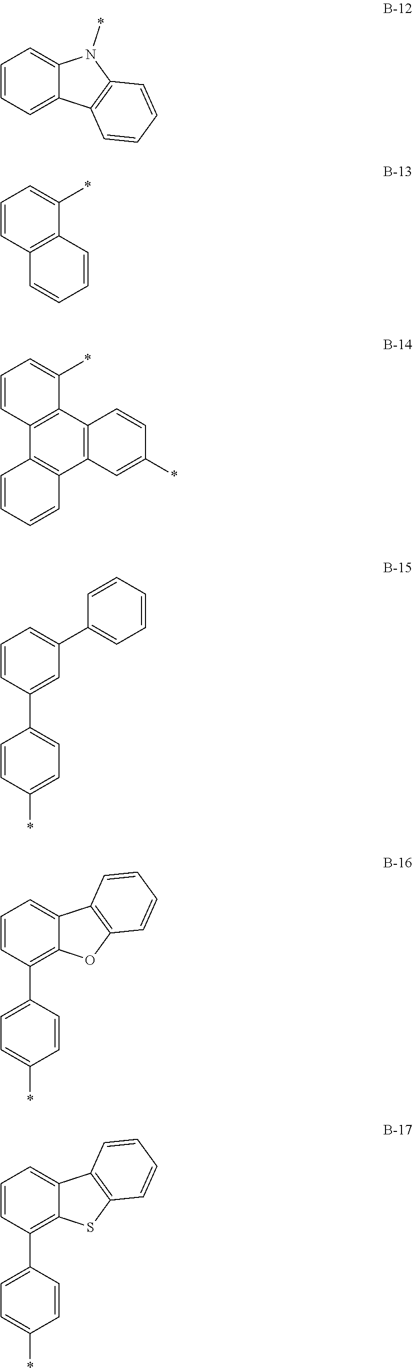 [SMILES: *n1c2ccccc2c2ccccc21 ; *c1cccc2ccccc12 ; *c1ccc2c(c1)c1ccccc1c1cccc(*)c12 ; *c1ccc(-c2cccc(-c3ccccc3)c2)cc1 ; *c1ccc(-c2cccc3c2oc2ccccc23)cc1 ; *c1ccc(-c2cccc3c2sc2ccccc23)cc1]

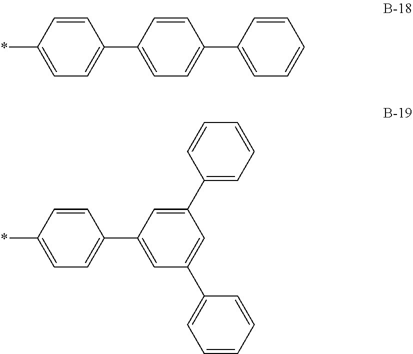 [SMILES: *c1ccc(-c2ccc(-c3ccccc3)cc2)cc1 ; *c1ccc(-c2cc(-c3ccccc3)cc(-c3ccccc3)c2)cc1]

In Groups I and II, * is a linking point.

The compound for an organic optoelectronic device represented by Chemical Formula 2 may be for example selected from compounds of Group 2, but is not limited thereto.

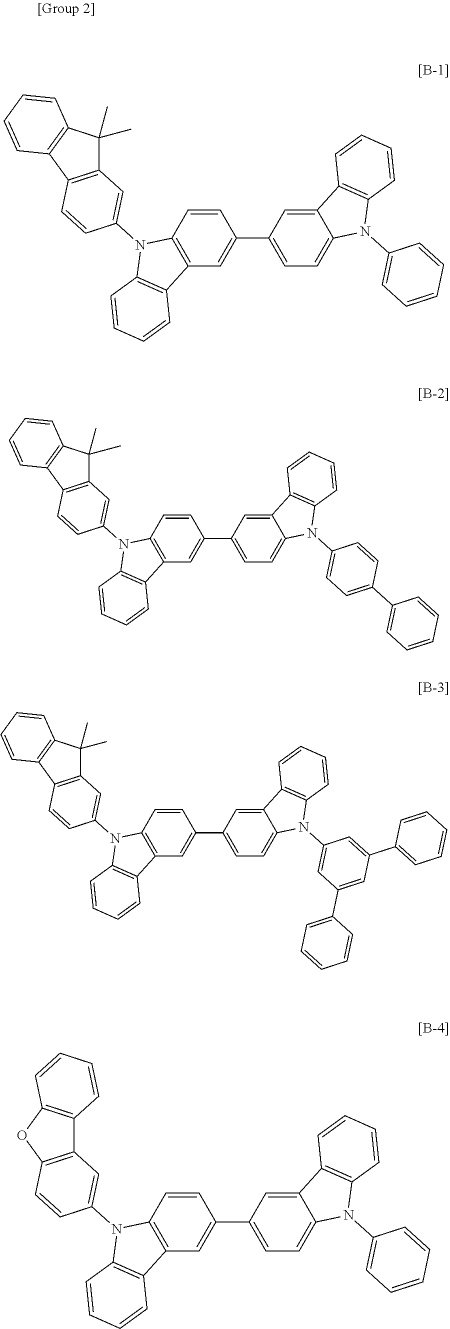 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1]

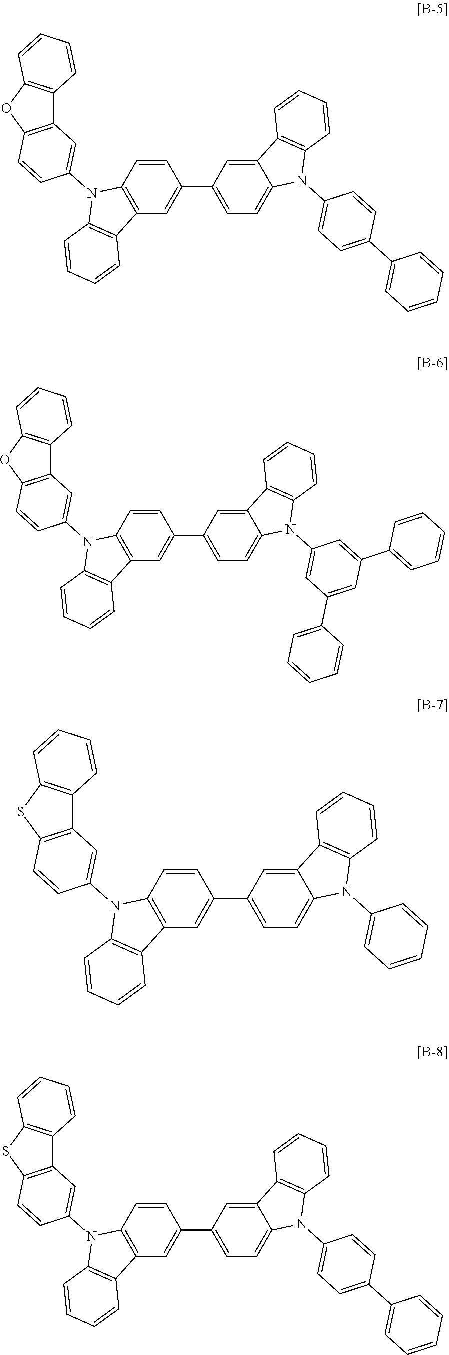 [SMILES: c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

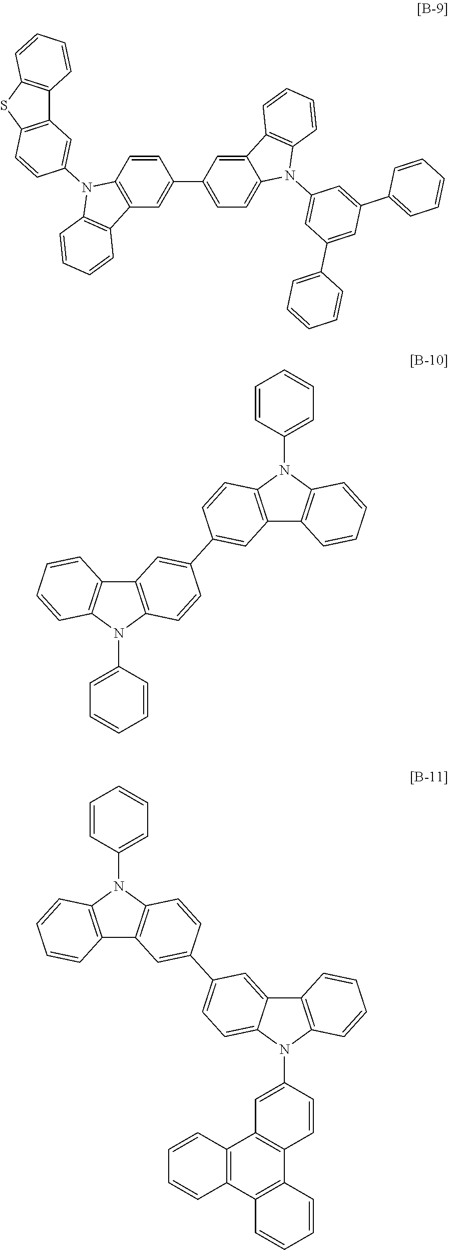 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

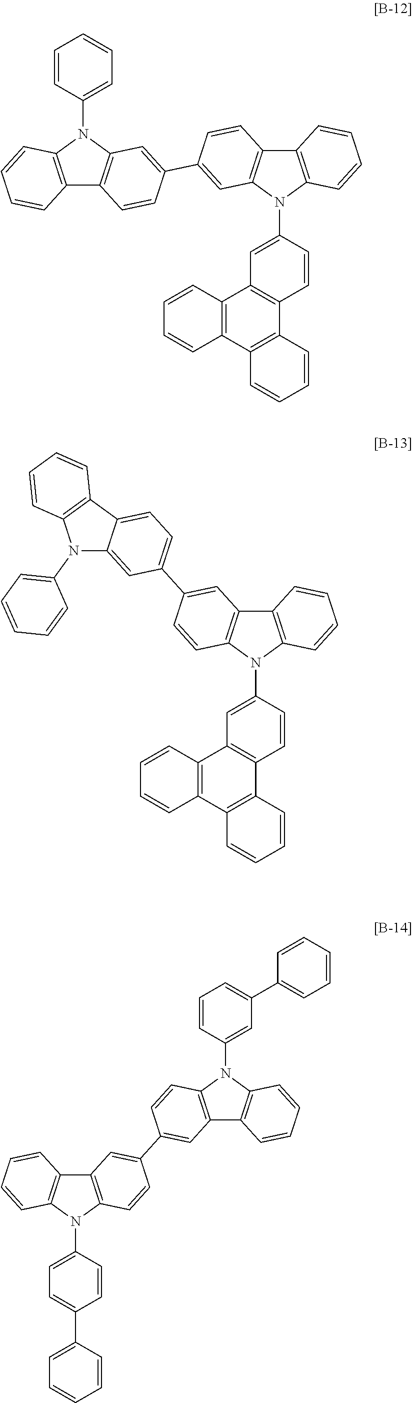 [SMILES: c1ccc(-n2c3ccccc3c3ccc(-c4ccc5c6ccccc6n(-c6ccc7c8ccccc8c8ccccc8c7c6)c5c4)cc32)cc1 ; c1ccc(-n2c3ccccc3c3ccc(-c4ccc5c(c4)c4ccccc4n5-c4ccc5c6ccccc6c6ccccc6c5c4)cc32)cc1 ; c1ccc(-c2ccc(-n3c4ccccc4c4cc(-c5ccc6c(c5)c5ccccc5n6-c5cccc(-c6ccccc6)c5)ccc43)cc2)cc1]

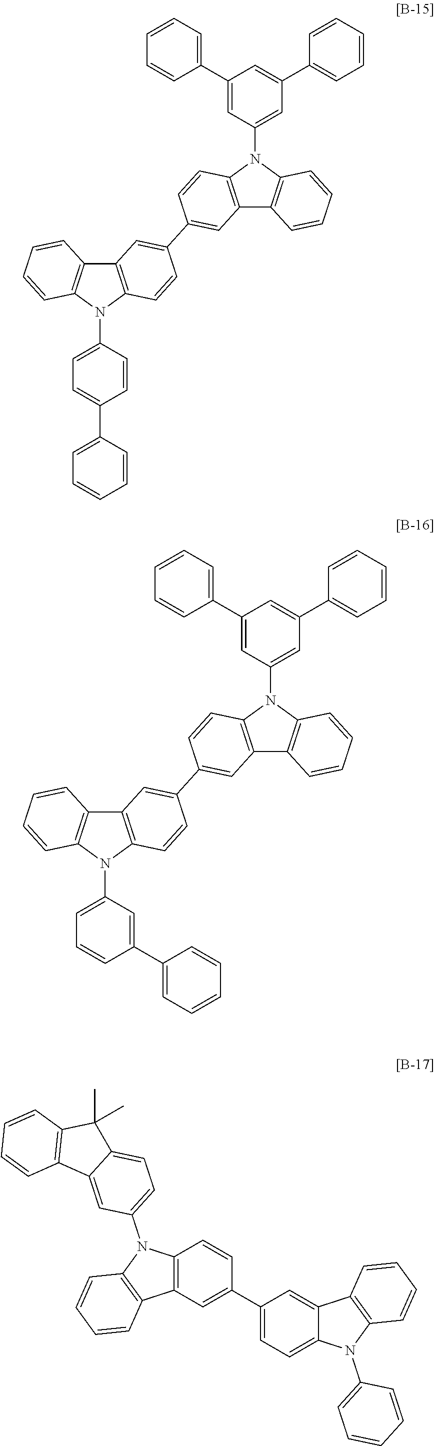 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21]

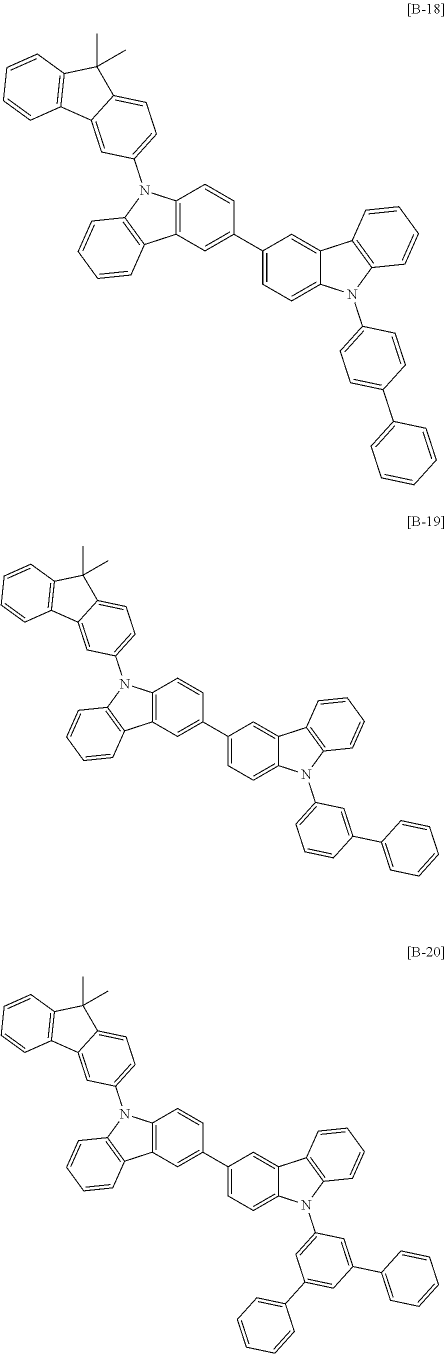 [SMILES: CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21]

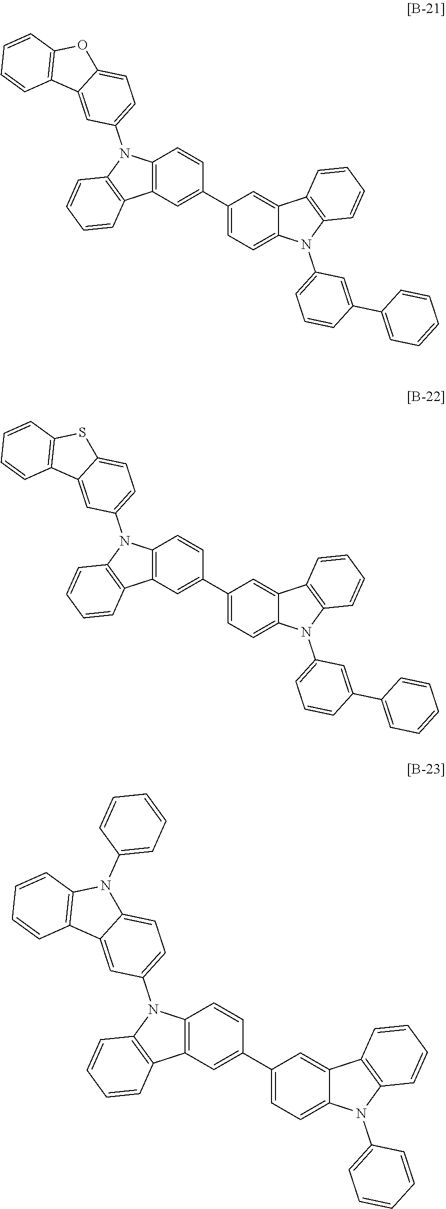 [SMILES: c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

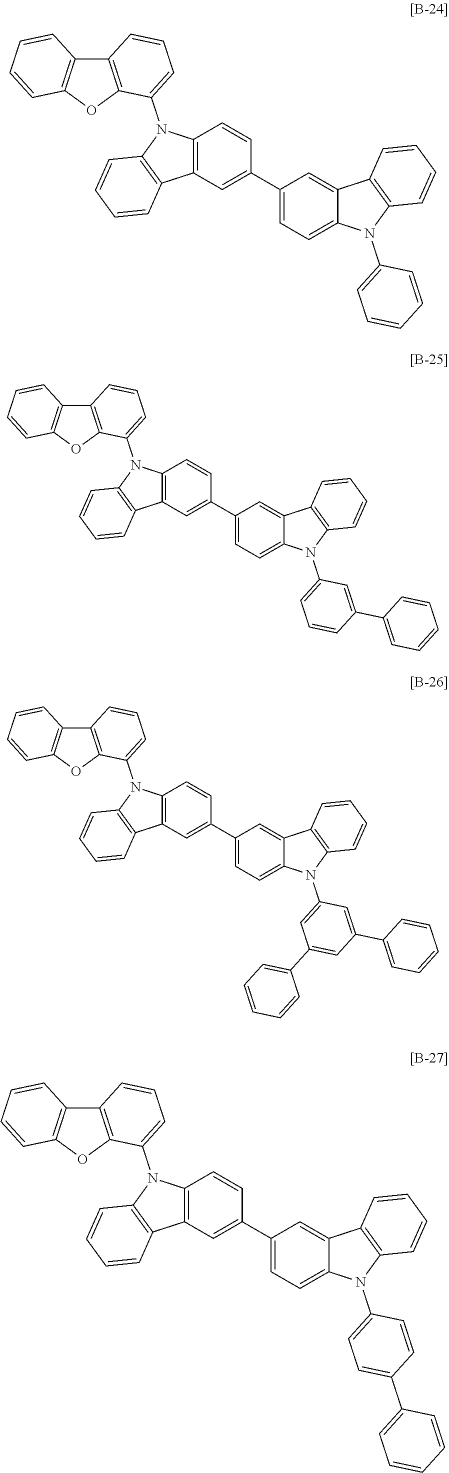 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1]

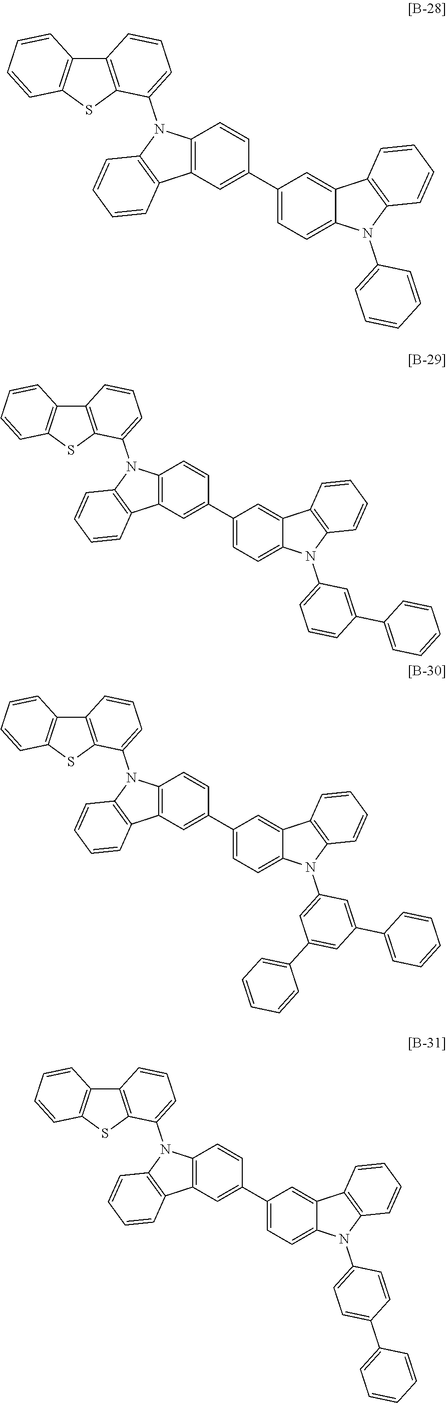 [SMILES: c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

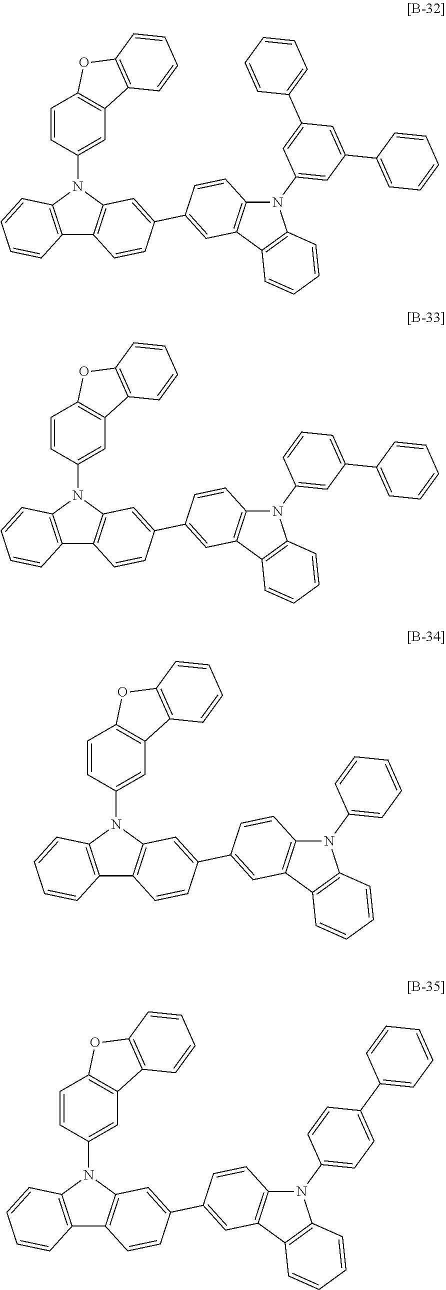 [SMILES: c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1]

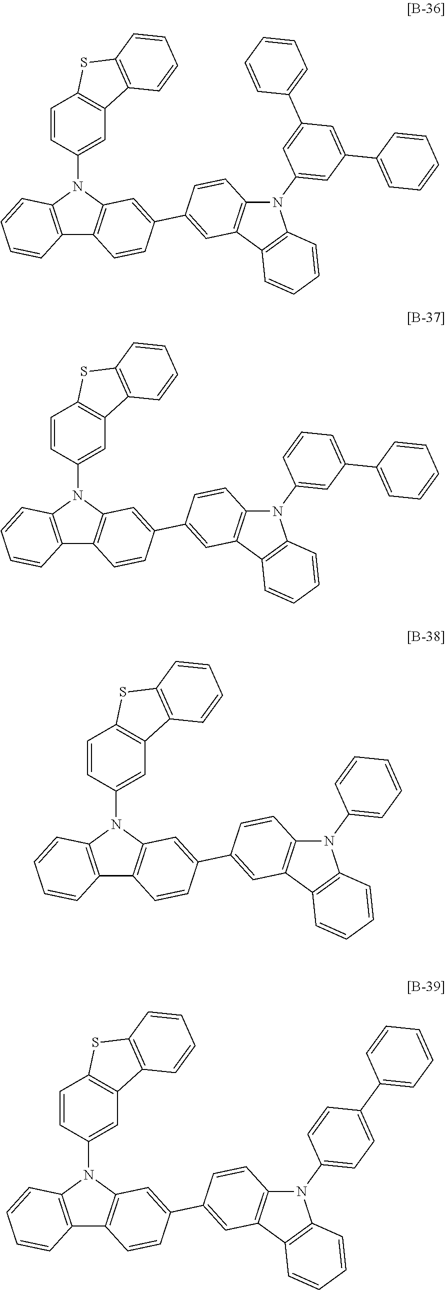 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1]

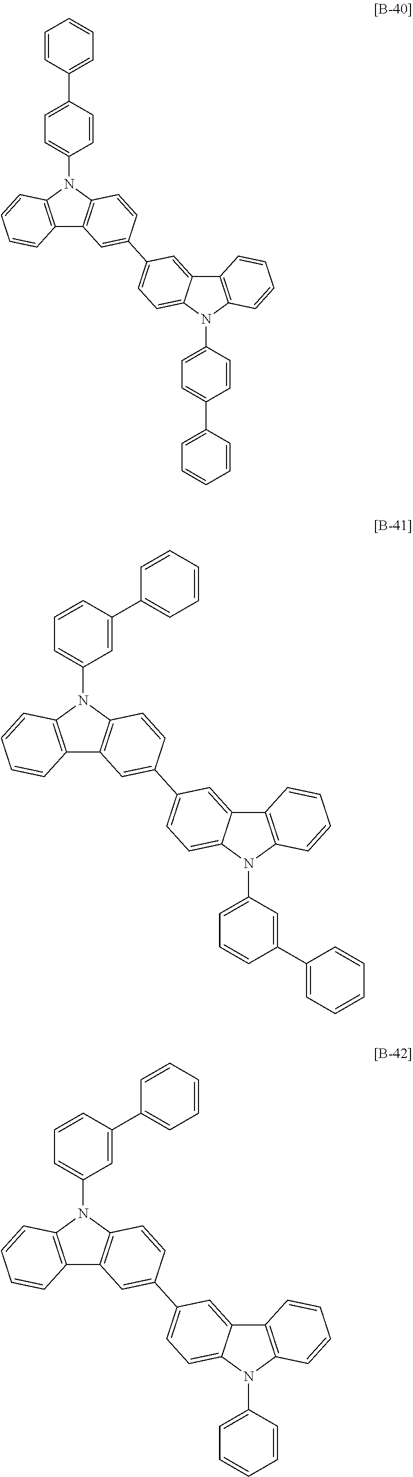 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1]

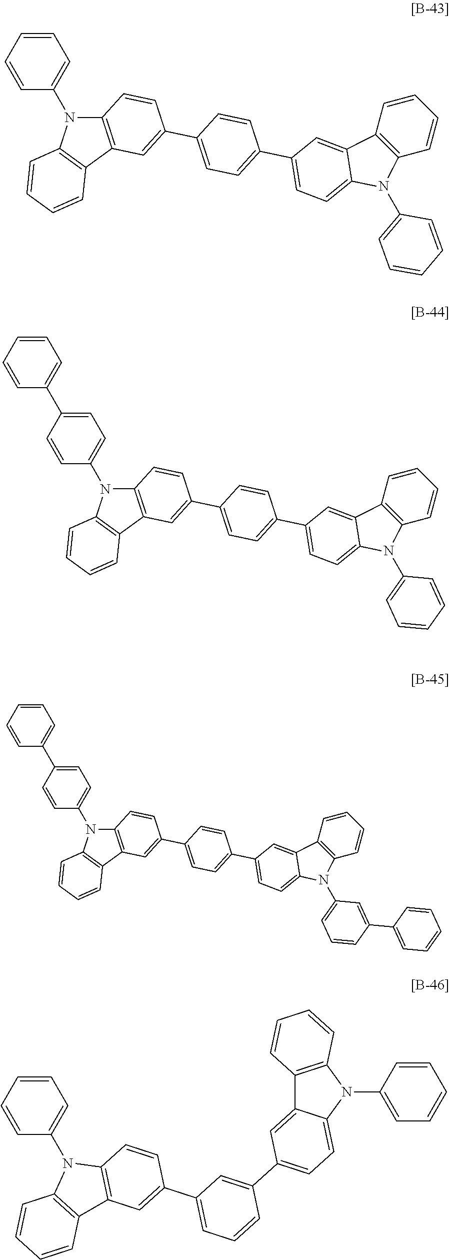 [SMILES: c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1]

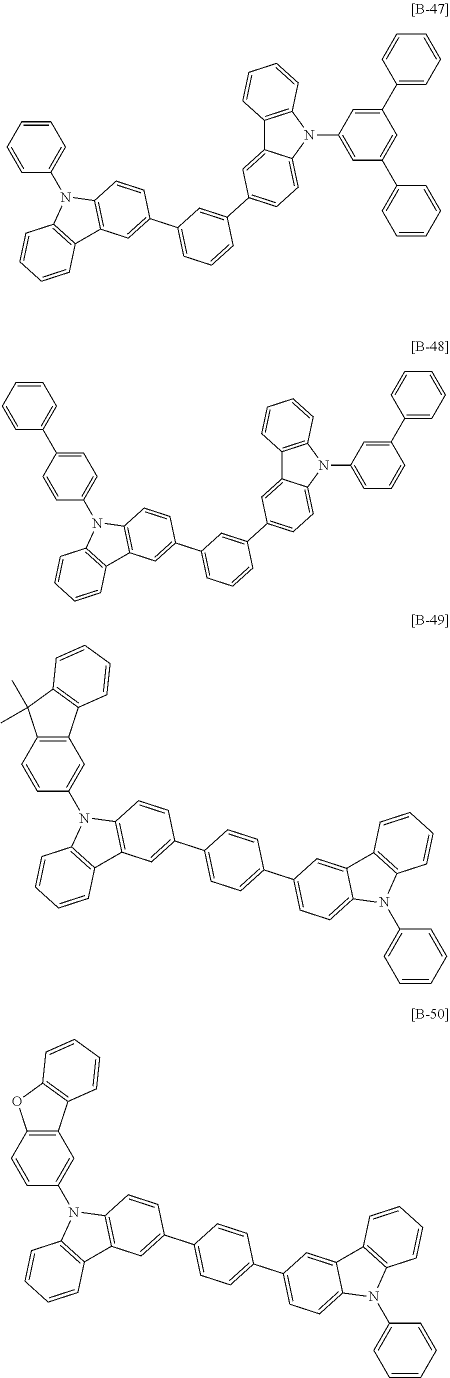 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21 ; c1ccc(-n2c3ccccc3c3cc(-c4ccc(-c5ccc6c(c5)c5ccccc5n6-c5ccc6oc7ccccc7c6c5)cc4)ccc32)cc1]

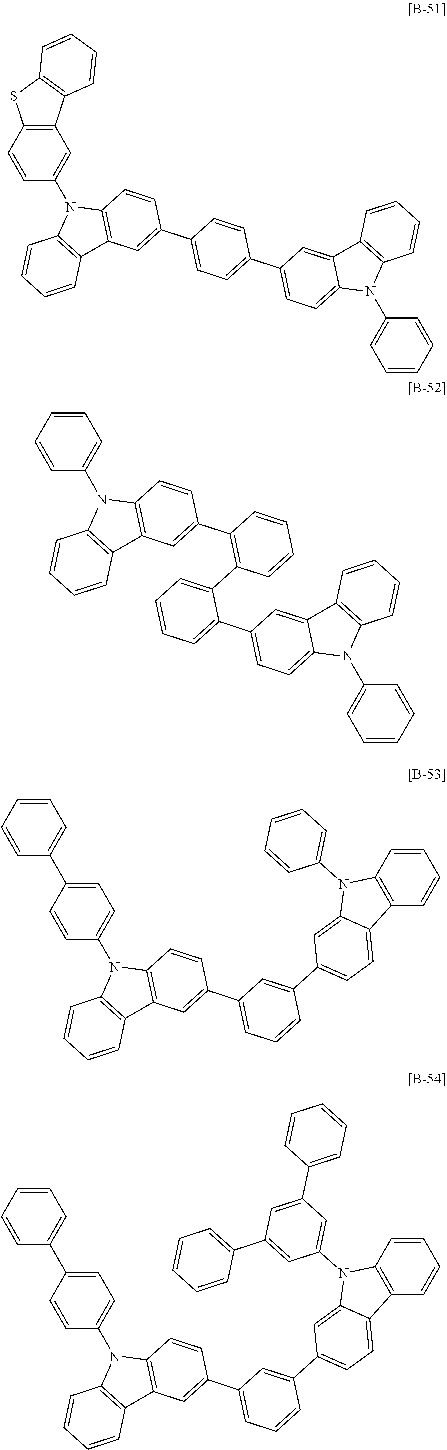 [SMILES: c1ccc(-n2c3ccccc3c3cc(-c4ccc(-c5ccc6c(c5)c5ccccc5n6-c5ccc6sc7ccccc7c6c5)cc4)ccc32)cc1 ; c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1]

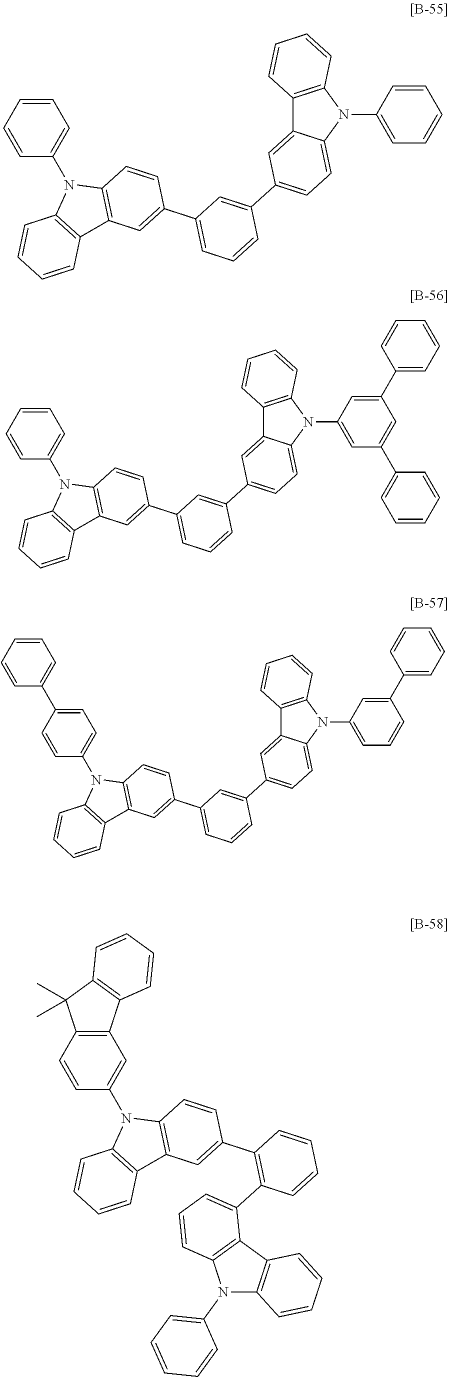 [SMILES: c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21]

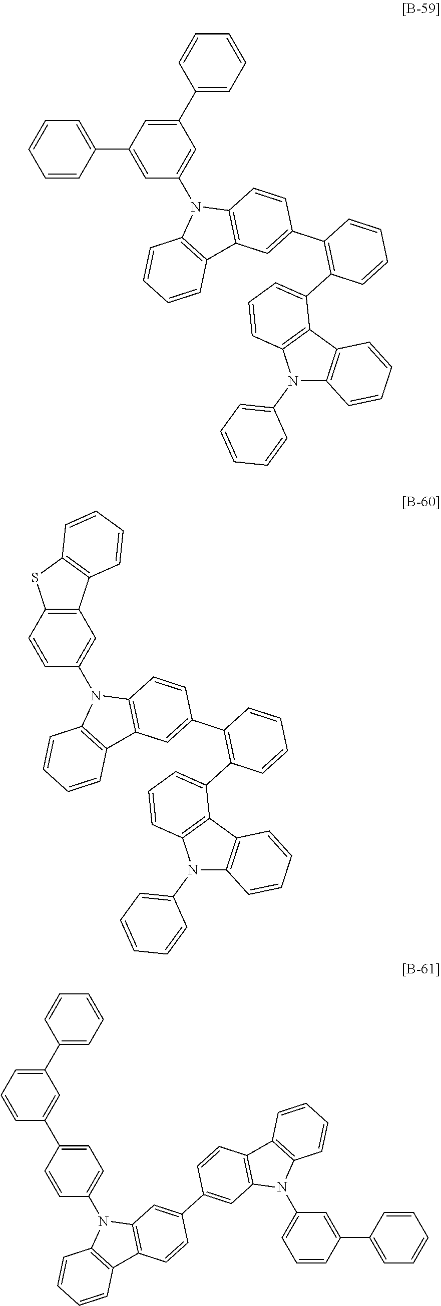 [SMILES: c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

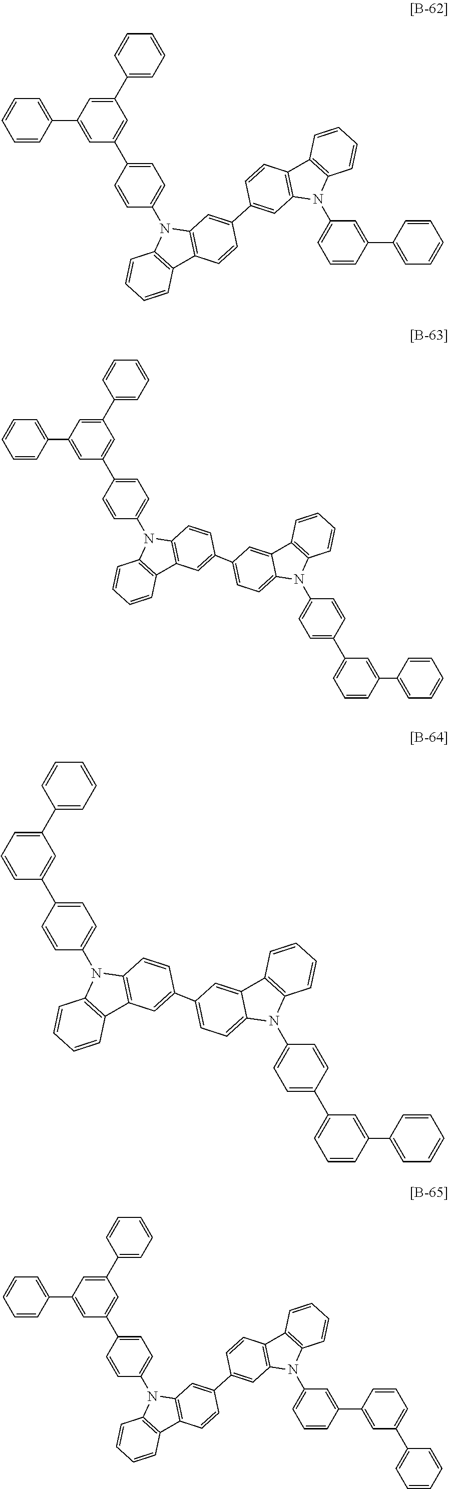 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cccc(-c3ccc(-n4c5ccccc5c5cc(-c6ccc7c(c6)c6ccccc6n7-c6ccc(-c7cccc(-c8ccccc8)c7)cc6)ccc54)cc3)c2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

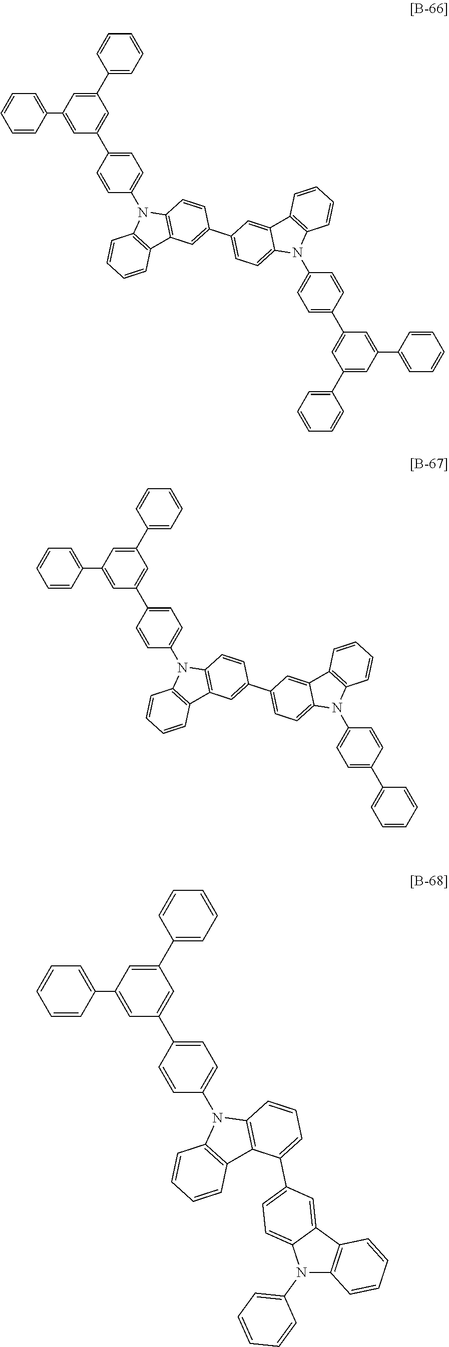 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

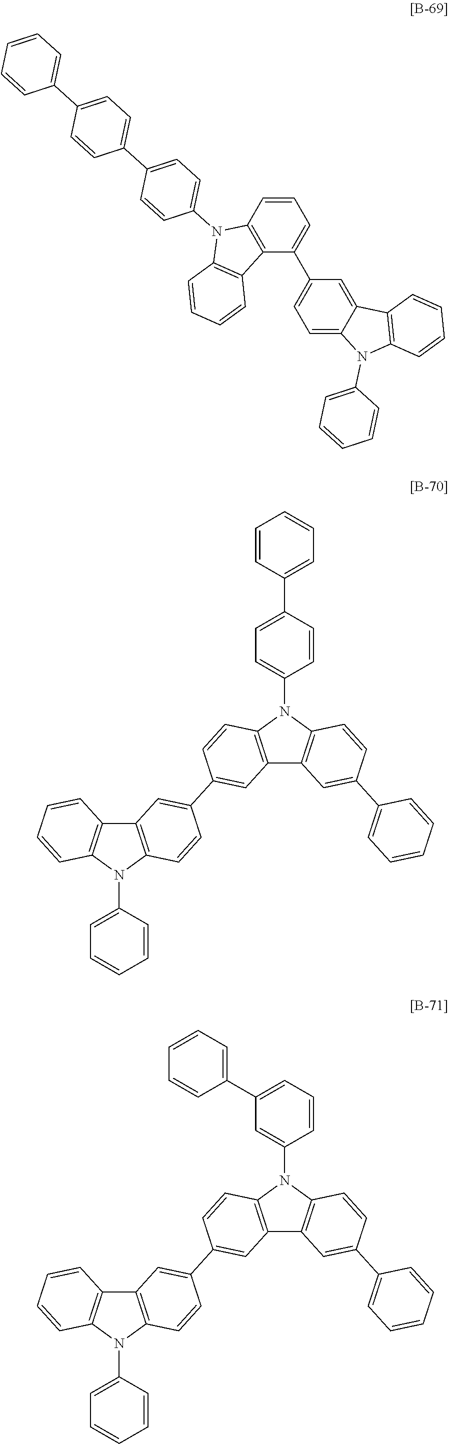 [SMILES: c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

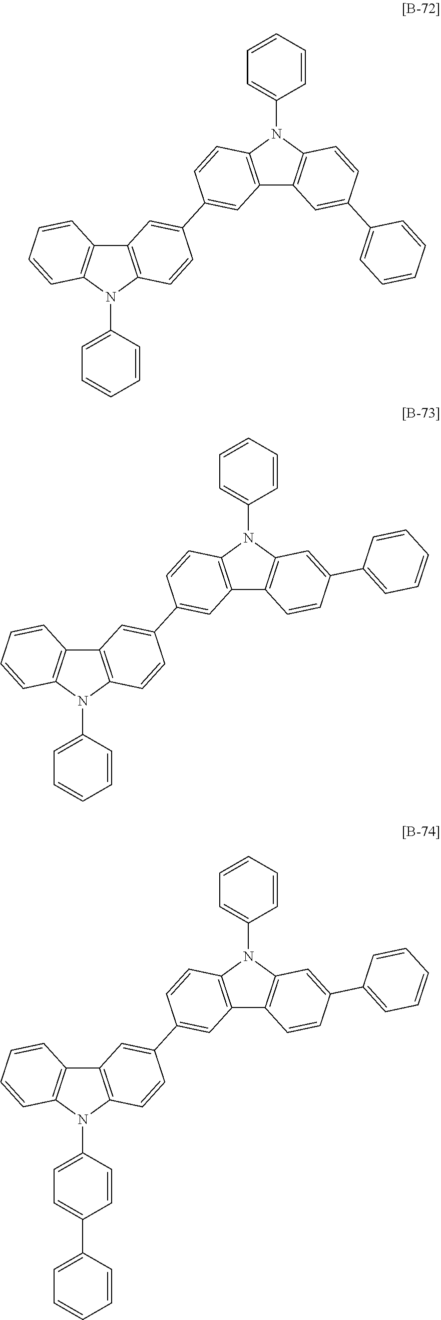 [SMILES: c1ccc(-c2ccc3c(c2)c2cc(-c4ccc5c(c4)c4ccccc4n5-c4ccccc4)ccc2n3-c2ccccc2)cc1 ; c1ccc(-c2ccc3c4cc(-c5ccc6c(c5)c5ccccc5n6-c5ccccc5)ccc4n(-c4ccccc4)c3c2)cc1 ; c1ccc(-c2ccc(-n3c4ccccc4c4cc(-c5ccc6c(c5)c5ccc(-c7ccccc7)cc5n6-c5ccccc5)ccc43)cc2)cc1]

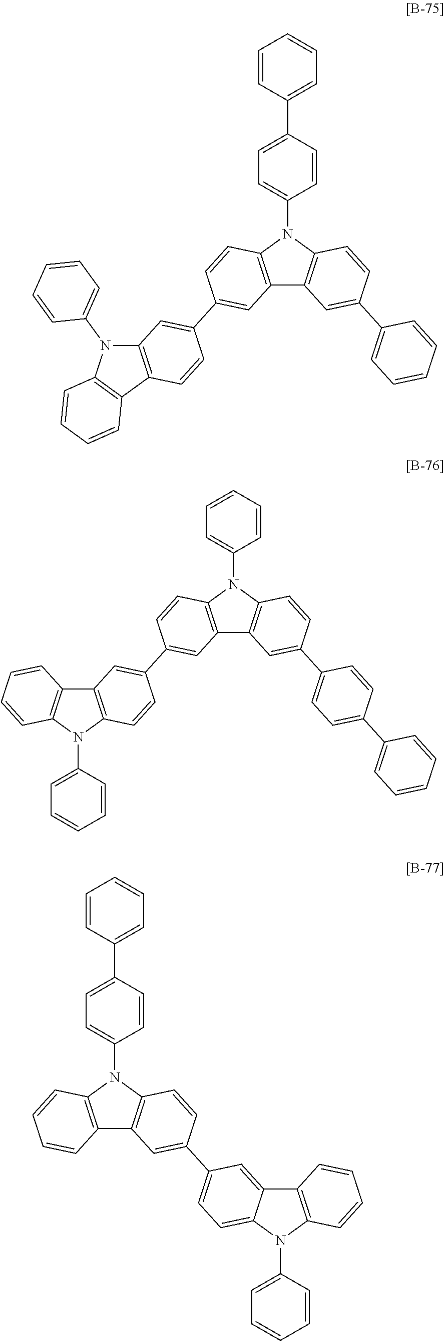 [SMILES: c1ccc(-c2ccc(-n3c4ccc(-c5ccccc5)cc4c4cc(-c5ccc6c7ccccc7n(-c7ccccc7)c6c5)ccc43)cc2)cc1 ; c1ccc(-c2ccc(-c3ccc4c(c3)c3cc(-c5ccc6c(c5)c5ccccc5n6-c5ccccc5)ccc3n4-c3ccccc3)cc2)cc1 ; c1ccc(-c2ccc(-n3c4ccccc4c4cc(-c5ccc6c(c5)c5ccccc5n6-c5ccccc5)ccc43)cc2)cc1]

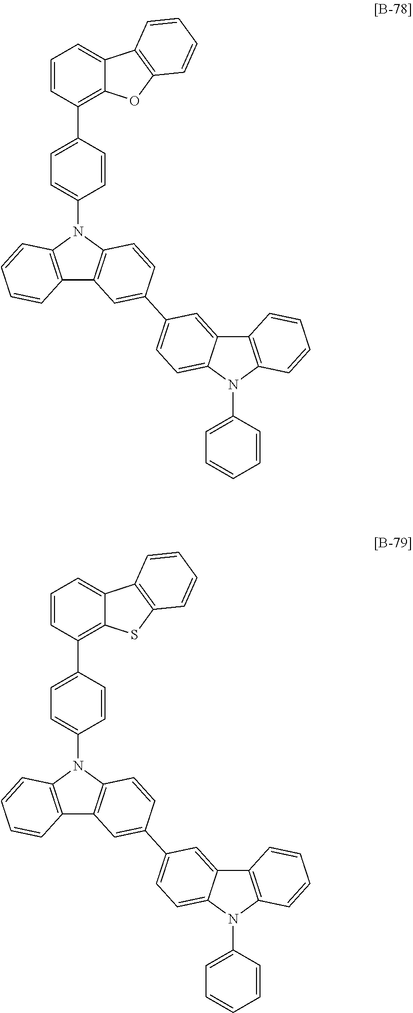 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

The first compound for an organic optoelectronic device and the second compound for an organic optoelectronic device may be prepared in various compositions by various combinations.

For example, when the composition of the present invention is used as a host of the light emitting layer 130, specifically a green phosphorescent host, a combination thereof ratio may be different depending on kinds or tendency of used dopants, and may be for example a weight ratio of about 1:9 to 9:1, specifically 1:9 to 8:2, 1:9 to 7:3, 1:9 to 6:4, 1:9 to 5:5, 2:8 to 8:2, 2:8 to 7:3, 2:8 to 6:4, or 2:8 to 5:5.

Specifically, the first compound for an organic optoelectronic device and the second compound for an organic optoelectronic device may be included in a weight ratio of 1:9 to 5:5, 2:8 to 5:5, or 3:7 to 5:5, and for example the first compound for an organic optoelectronic device and the second compound for an organic optoelectronic device may be included in a weight ratio of 5:5. Within the ranges, efficiency and life-span may be simultaneously improved.

Within the ranges, bipolar characteristics may be effectively embodied and thus efficiency and life-span may be simultaneously improved.

A composition according to an example embodiment of the present invention includes the compound represented by Chemical Formula 1-I or Chemical Formula 1-II as a first host and the compound represented by Chemical Formula C-8 or Chemical Formula C-17 of Group I as a second host.

In addition, the first host represented by Chemical Formula 1A, or Chemical Formula 1B and the second host represented by Chemical Formula C-8 or Chemical Formula C-17 of Group I may be included.

In addition, the first host represented by Chemical Formula 1-1 and the second host represented by Chemical Formula C-8 or Chemical Formula C-17 of Group I may be included.

For example, *-L¹-Ar¹ and *-L²-Ar² of Chemical Formula 2 may be selected from B-1, B-2, B-3, and B-16 of Group II.

The light emitting layer 130 may further include a dopant. The dopant is mixed with the host in a small amount to cause light emission, and may be generally a material such as a metal complex that emits light by multiple excitation into a triplet or more. The dopant may be for example an inorganic, organic, or organic/inorganic compound and one or more kinds thereof may be used.

The dopant may be a red, green, or blue dopant, for example a phosphorescent dopant. Examples of the phosphorescent dopant may be an organometallic compound including Ir, Pt, Os, Ti, Zr, Hf, Eu, Tb, Tm, Fe, Co, Ni, Ru, Rh, Pd, or a combination thereof. The phosphorescent dopant may be for example a compound represented by Chemical Formula Z, but is not limited thereto.

L₂MX [Chemical Formula Z]

In Chemical Formula Z, M is a metal, and L and X are the same or different, and are a ligand to form a complex compound with M.

M may be for example Ir, Pt, Os, Ti, Zr, Hf, Eu, Tb, Tm, Fe, Co, Ni, Ru, Rh, Pd, or a combination thereof and L and X may be, for example a bidendate ligand.

The electron transport layer 140 is a layer that facilitates electron transport from the cathode 110 into the light emitting layer 130 and may include the compound represented by Chemical Formula 3.

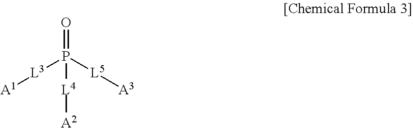

In Chemical Formula 3,

L³ to L⁵ are independently a single bond, a substituted or unsubstituted C6 to C30 arylene group, a substituted or unsubstituted C2 to C30 heteroarylene group, or a combination thereof,

A¹ to A³ are independently a substituted or unsubstituted C6 to C30 aryl group, a substituted or unsubstituted C2 to C30 heterocyclic group, or a combination thereof,

A¹ to A³ are independently present or adjacent groups are linked with each other to form at least one of a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring, and

when A¹ to A³ are independently present, at least one of A¹ to A³ is a substituted or unsubstituted fused aryl group or a substituted or unsubstituted fused heterocyclic group.

When A¹ to A³ are independently present in Chemical Formula 3 of the present invention, at least one of A¹ to A³ may be a substituted or unsubstituted fused aryl group or a substituted or unsubstituted fused heterocyclic group and thereby, electron characteristics of a phosphorus atom may be expanded to a fused substituent moiety and thus electron injection and transport characteristics may be effectively improved compared with a structure having a non-fused substituent.

The “substituted” refers to replacement of at least one hydrogen by deuterium, a C1 to C4 alkyl group, a C6 to C18 aryl group, or a C2 to C30 heteroaryl group.

In one example of the present invention, “substituted” in Chemical Formula 3 may refer to replacement of at least one hydrogen by deuterium, a C1 to C4 alkyl group, a C6 to C20 aryl group, or C2 to C20 heteroaryl group, specifically the “substituted” may refer to replacement of at least one hydrogen by deuterium, a C1 to C4 alkyl group, a phenyl group, a biphenyl group, a naphthyl group, a terphenyl group, an anthracenyl group, a phenanthrenyl group, a fluorenyl group, a triphenylene group, a fluoranthenyl group, a carbazolyl group, a dibenzofuranyl group, a dibenzothiophenyl group, a pyridinyl group, a pyrimidinyl group, a triazinyl group, a quinolinyl group, or an isoquinolinyl group, an azaphenanthrenyl group, or a phenanthrolinyl group.

In one example of the present invention, when A¹ to A³ are independently present, at least one of A¹ to A³ may be a substituted or unsubstituted fused aryl group or a substituted or unsubstituted fused heterocyclic group, and the substituted or unsubstituted fused aryl group or the substituted or unsubstituted fused heterocyclic group may be selected from substituents of Group III.

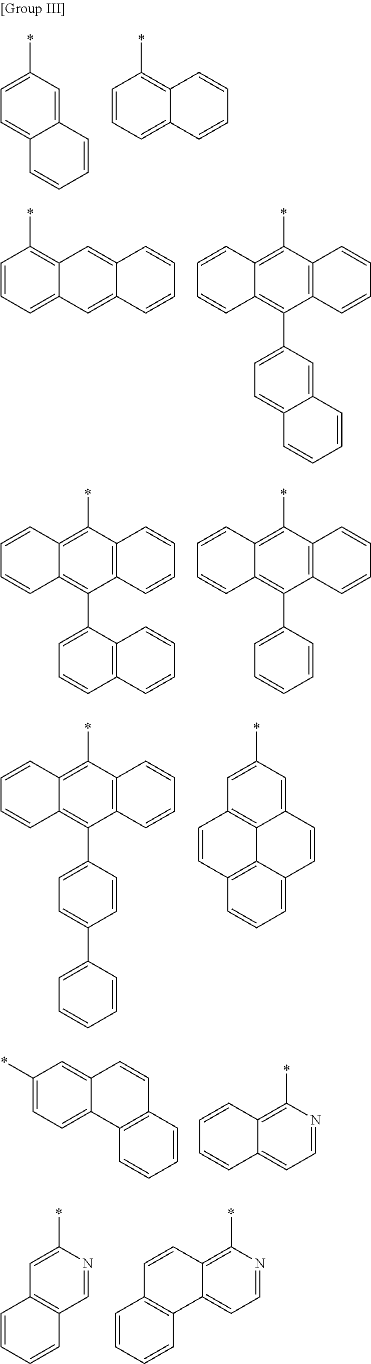

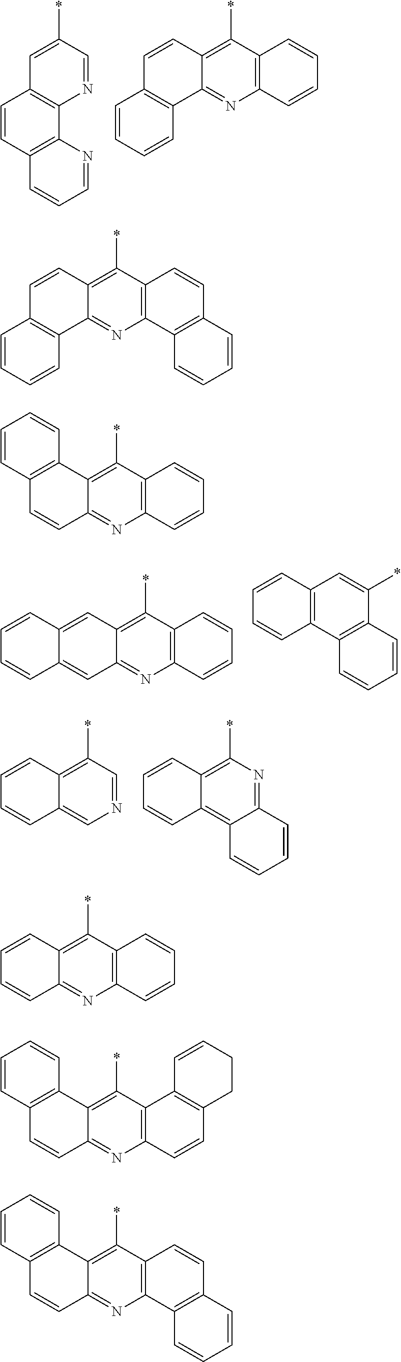

In addition, in one example of the present invention, adjacent groups of A¹ to A³ may be linked with each other to form at least one of a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring, and for example to form a substituted or unsubstituted aromatic monocyclic 7-membered ring as follows.

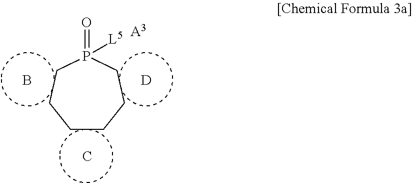

In Chemical Formula 3a, L⁵ and A³ are the same as described above, and B, C, and D may be formed from L³, L⁴, A¹, and A² and may be a substituted or unsubstituted C6 to C30 aryl or a substituted or unsubstituted C2 to C30 heterocyclic while sharing a septangular core and two carbons.

In one example of the present invention, B, C, and D may independently be a substituted or unsubstituted phenyl, a substituted or unsubstituted naphthyl, a substituted or unsubstituted phenanthrene, a substituted or unsubstituted triphenylene, a substituted or unsubstituted pyridine, a substituted or unsubstituted pyrimidine, a substituted or unsubstituted quinoline, or a substituted or unsubstituted isoquinoline, and in more specific examples, they may be selected from substituted or unsubstituted moieties of Group IV.

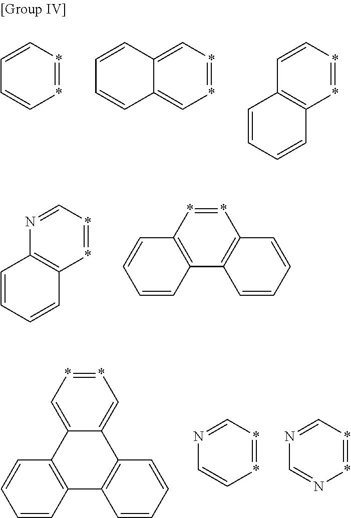

In Group IV, *'s indicate carbon shared with the septangular core of Chemical Formula 3a.

In specific examples of the present invention, B, C, and D may independently be a substituted or unsubstituted phenyl or a substituted or unsubstituted naphthyl.

In one example of the present invention, L³ to L⁵ may independently be a single bond, a substituted or unsubstituted phenylene group, a substituted or unsubstituted biphenylene group, or a substituted or unsubstituted pyridylene group.

The compound for an organic optoelectronic device represented by Chemical Formula 3 may be for example compounds of Group 3, but is not limited thereto.

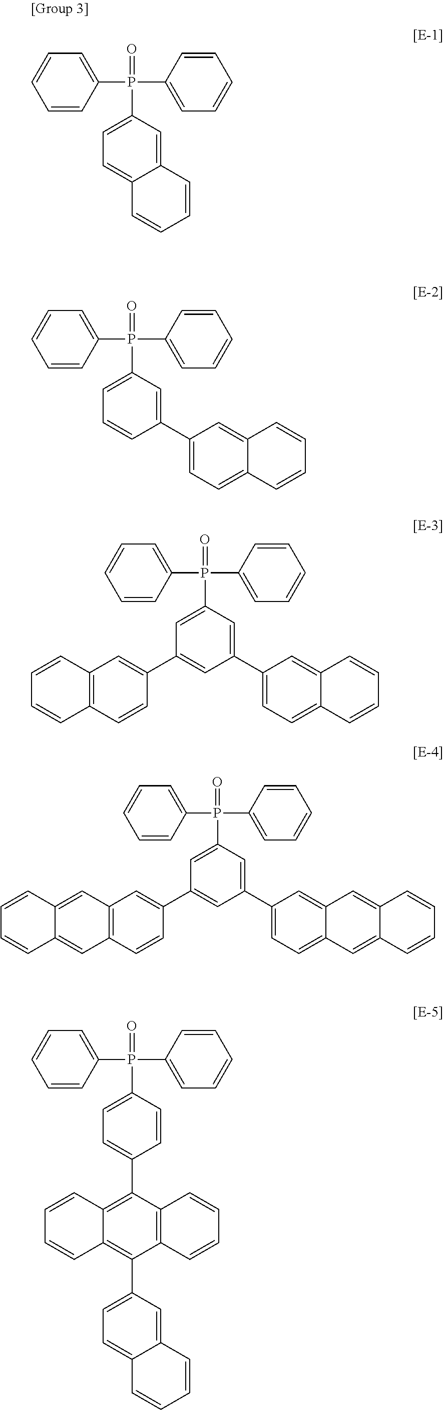 [SMILES: O=P(c1ccccc1)(c1ccccc1)c1ccc2ccccc2c1 ; O=P(c1ccccc1)(c1ccccc1)c1cccc(-c2ccc3ccccc3c2)c1 ; O=P(c1ccccc1)(c1ccccc1)c1cc(-c2ccc3ccccc3c2)cc(-c2ccc3ccccc3c2)c1 ; O=P(c1ccccc1)(c1ccccc1)c1cc(-c2ccc3cc4ccccc4cc3c2)cc(-c2ccc3cc4ccccc4cc3c2)c1 ; O=P(c1ccccc1)(c1ccccc1)c1ccc(-c2c3ccccc3c(-c3ccc4ccccc4c3)c3ccccc23)cc1]

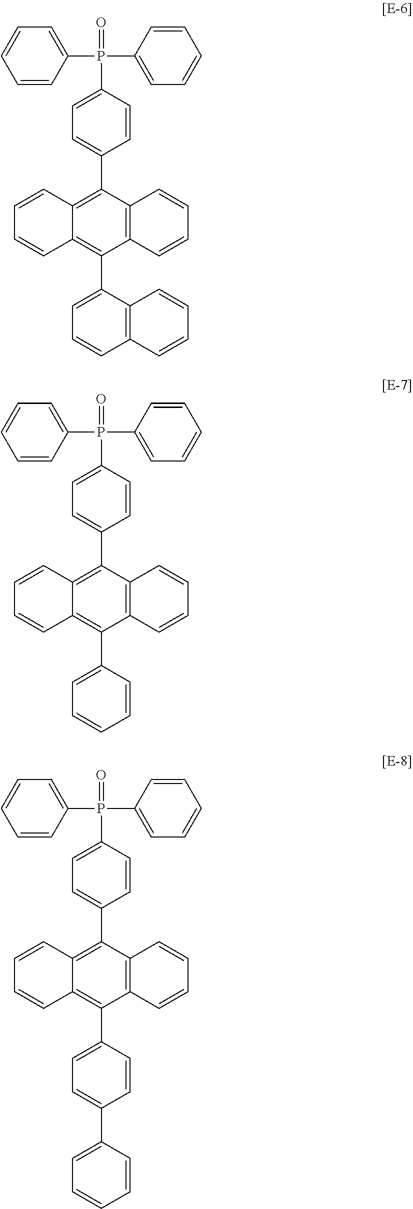

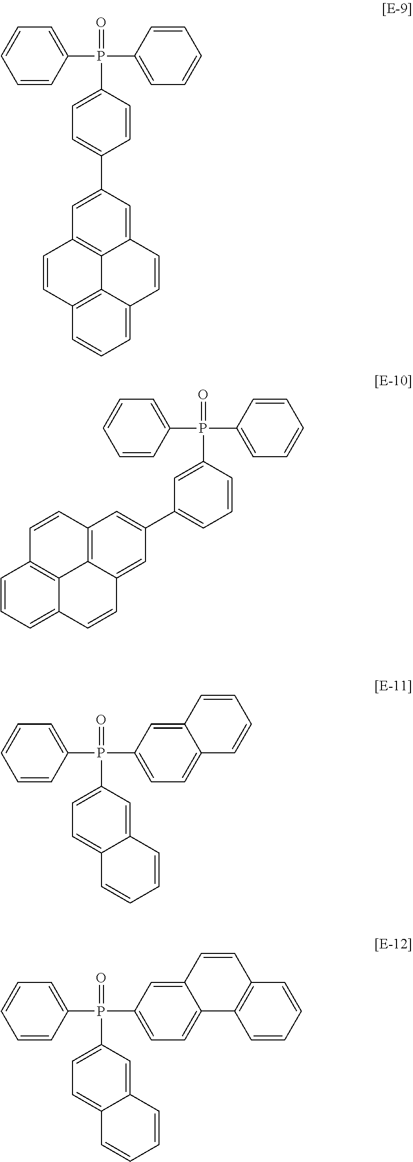 [SMILES: O=P(c1ccccc1)(c1ccccc1)c1ccc(-c2cc3ccc4cccc5ccc(c2)c3c45)cc1 ; O=P(c1ccccc1)(c1ccccc1)c1cccc(-c2cc3ccc4cccc5ccc(c2)c3c45)c1 ; O=P(c1ccccc1)(c1ccc2ccccc2c1)c1ccc2ccccc2c1 ; O=P(c1ccccc1)(c1ccc2ccccc2c1)c1ccc2c(ccc3ccccc32)c1]

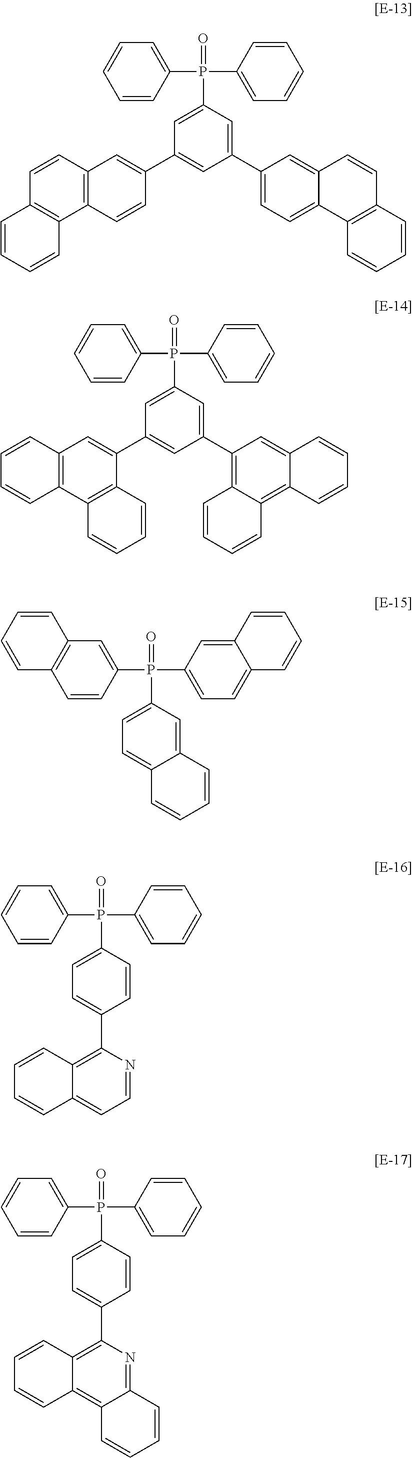 [SMILES: O=P(c1ccccc1)(c1ccccc1)c1cc(-c2ccc3c(ccc4ccccc43)c2)cc(-c2ccc3c(ccc4ccccc43)c2)c1 ; O=P(c1ccccc1)(c1ccccc1)c1cc(-c2cc3ccccc3c3ccccc23)cc(-c2cc3ccccc3c3ccccc23)c1 ; O=P(c1ccc2ccccc2c1)(c1ccc2ccccc2c1)c1ccc2ccccc2c1 ; O=P(c1ccccc1)(c1ccccc1)c1ccc(-c2nccc3ccccc23)cc1 ; O=P(c1ccccc1)(c1ccccc1)c1ccc(-c2nc3ccccc3c3ccccc23)cc1]

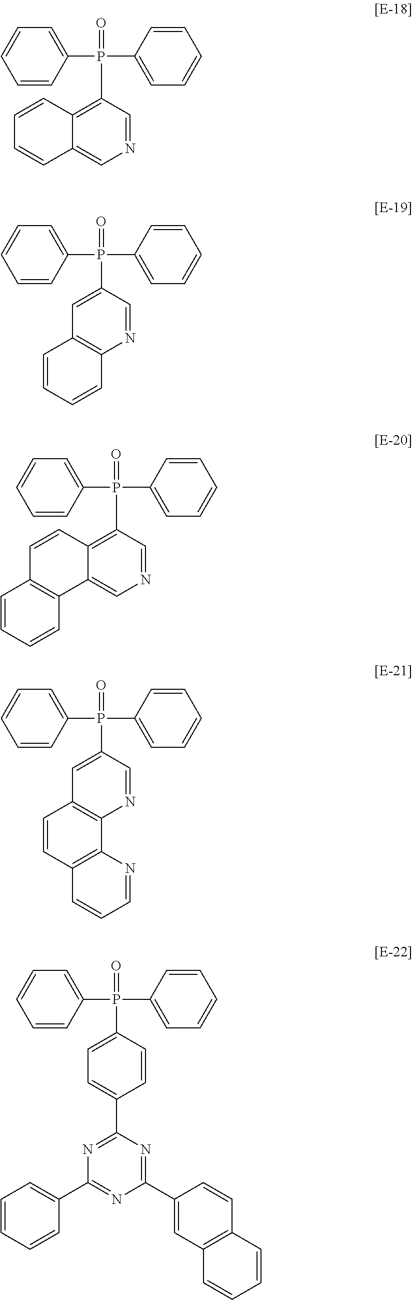

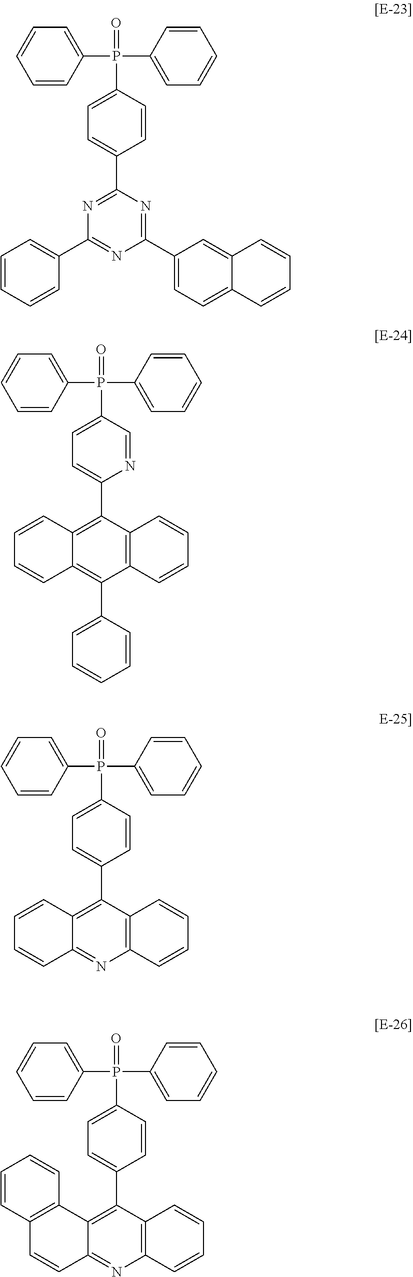 [SMILES: O=P(c1ccccc1)(c1ccccc1)c1ccc(-c2nc(-c3ccccc3)nc(-c3ccc4ccccc4c3)n2)cc1 ; O=P(c1ccccc1)(c1ccccc1)c1ccc(-c2c3ccccc3c(-c3ccccc3)c3ccccc23)nc1 ; O=P(c1ccccc1)(c1ccccc1)c1ccc(-c2c3ccccc3nc3ccccc23)cc1 ; O=P(c1ccccc1)(c1ccccc1)c1ccc(-c2c3ccccc3nc3ccc4ccccc4c23)cc1]

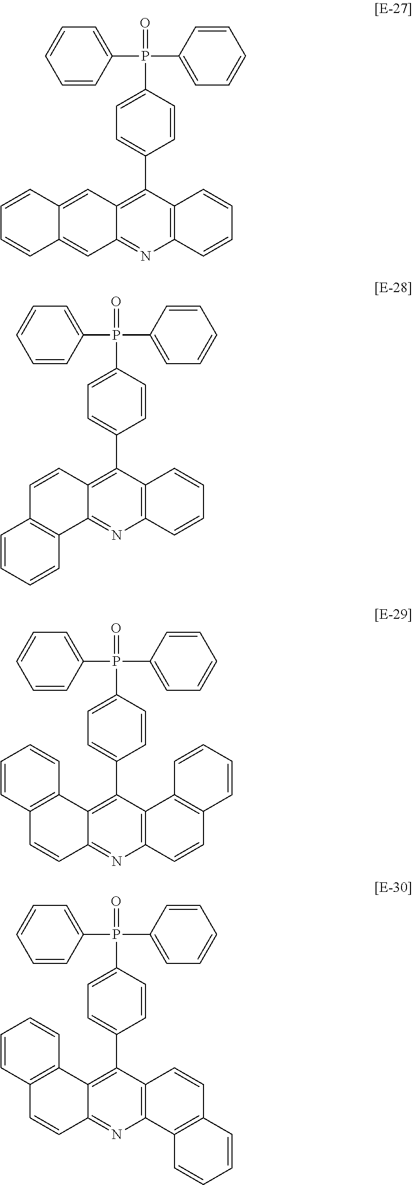

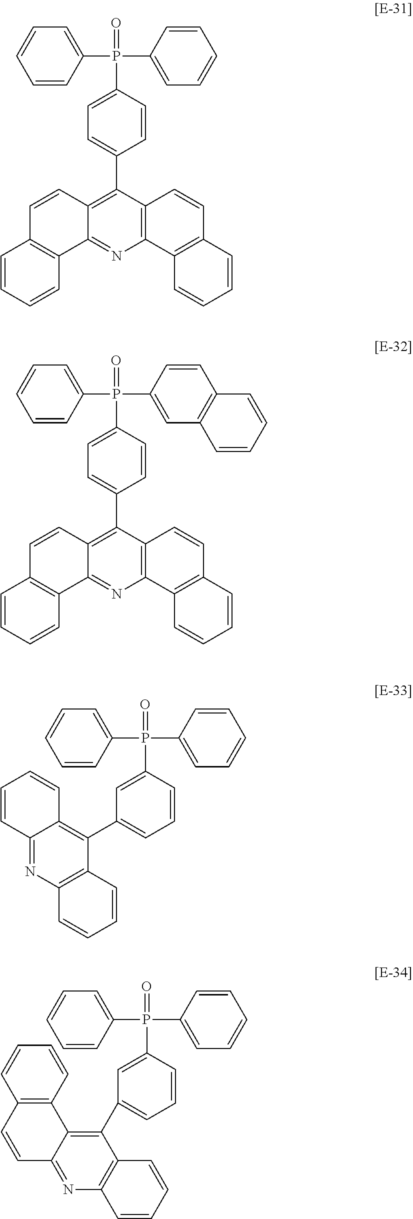 [SMILES: O=P(c1ccccc1)(c1ccccc1)c1ccc(-c2c3ccc4ccccc4c3nc3c2ccc2ccccc23)cc1 ; O=P(c1ccccc1)(c1ccc(-c2c3ccc4ccccc4c3nc3c2ccc2ccccc23)cc1)c1ccc2ccccc2c1 ; O=P(c1ccccc1)(c1ccccc1)c1cccc(-c2c3ccccc3nc3ccccc23)c1 ; O=P(c1ccccc1)(c1ccccc1)c1cccc(-c2c3ccccc3nc3ccc4ccccc4c23)c1]

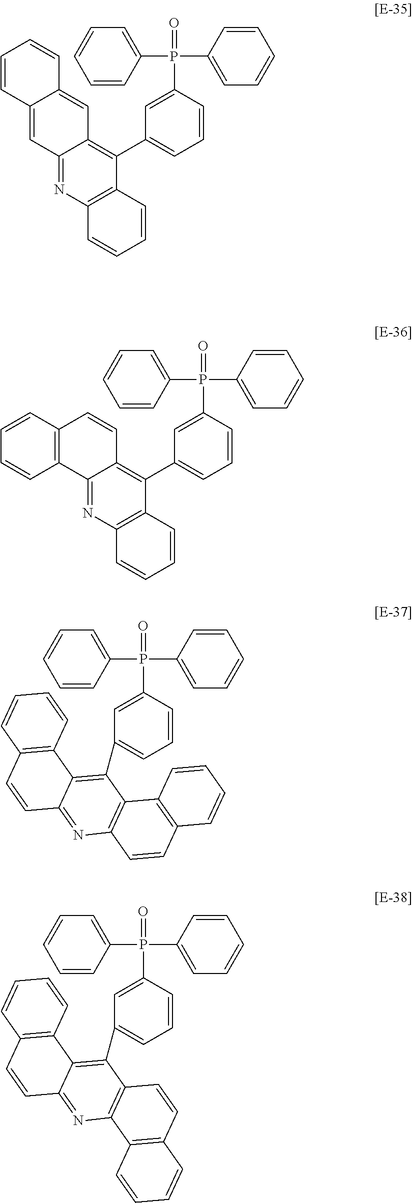

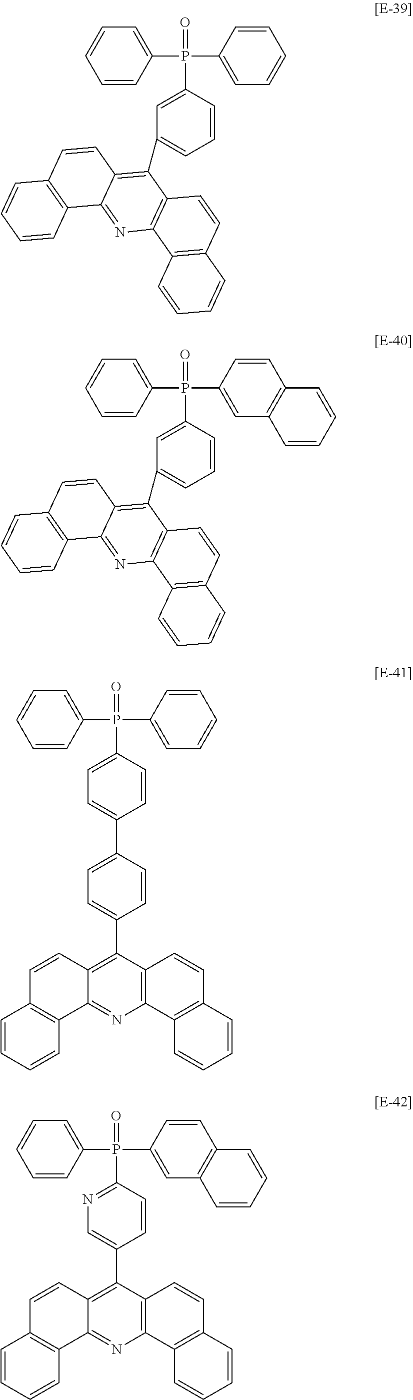

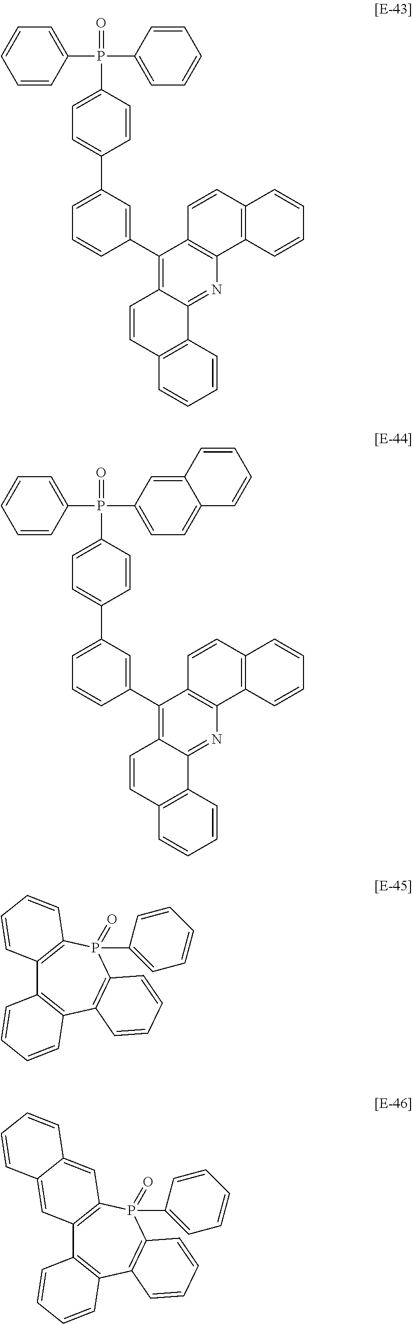

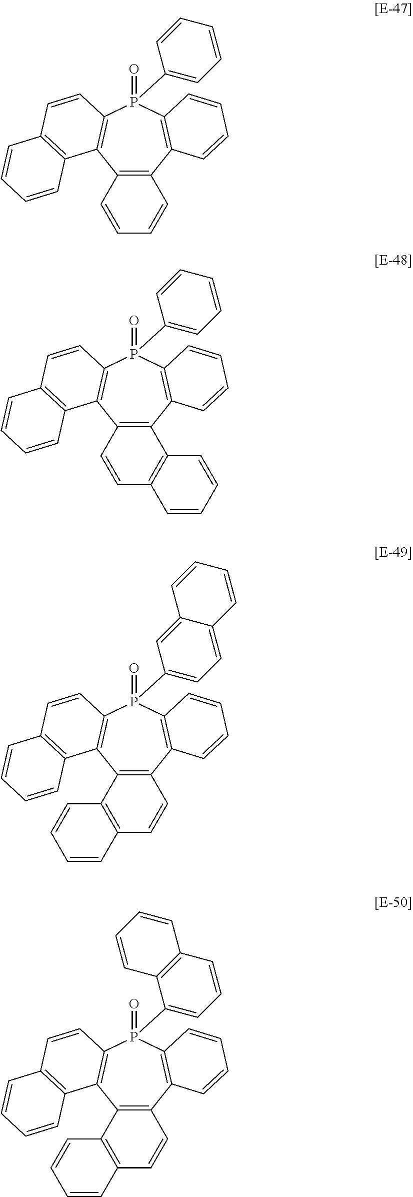 [SMILES: O=P1(c2ccccc2)c2ccccc2-c2ccccc2-c2c1ccc1ccccc21 ; O=P1(c2ccccc2)c2ccccc2-c2c(ccc3ccccc23)-c2c1ccc1ccccc21 ; O=P1(c2ccc3ccccc3c2)c2ccccc2-c2ccc3ccccc3c2-c2c1ccc1ccccc21 ; O=P1(c2cccc3ccccc23)c2ccccc2-c2ccc3ccccc3c2-c2c1ccc1ccccc21]

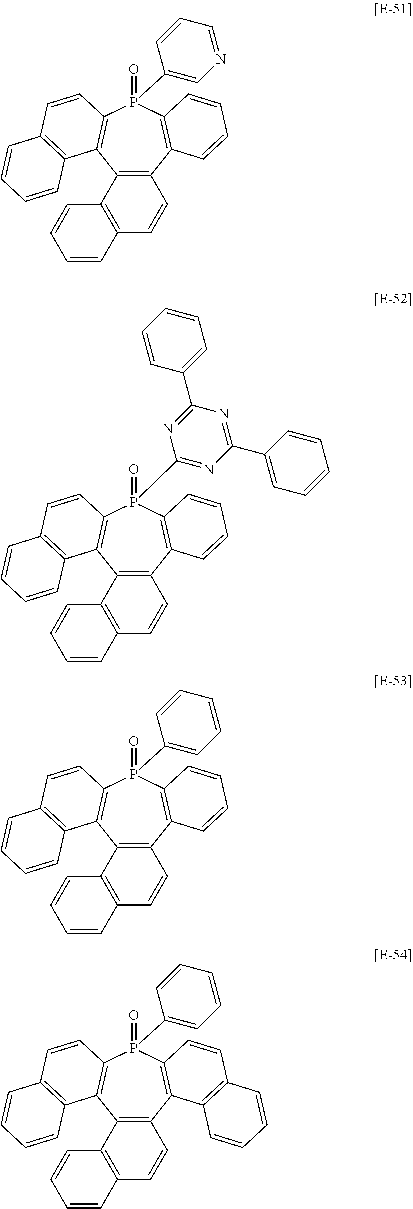 [SMILES: O=P1(c2cccnc2)c2ccccc2-c2ccc3ccccc3c2-c2c1ccc1ccccc21 ; O=P1(c2nc(-c3ccccc3)nc(-c3ccccc3)n2)c2ccccc2-c2ccc3ccccc3c2-c2c1ccc1ccccc21 ; O=P1(c2ccccc2)c2ccccc2-c2ccc3ccccc3c2-c2c1ccc1ccccc21 ; O=P1(c2ccccc2)c2ccc3ccccc3c2-c2ccc3ccccc3c2-c2c1ccc1ccccc21]

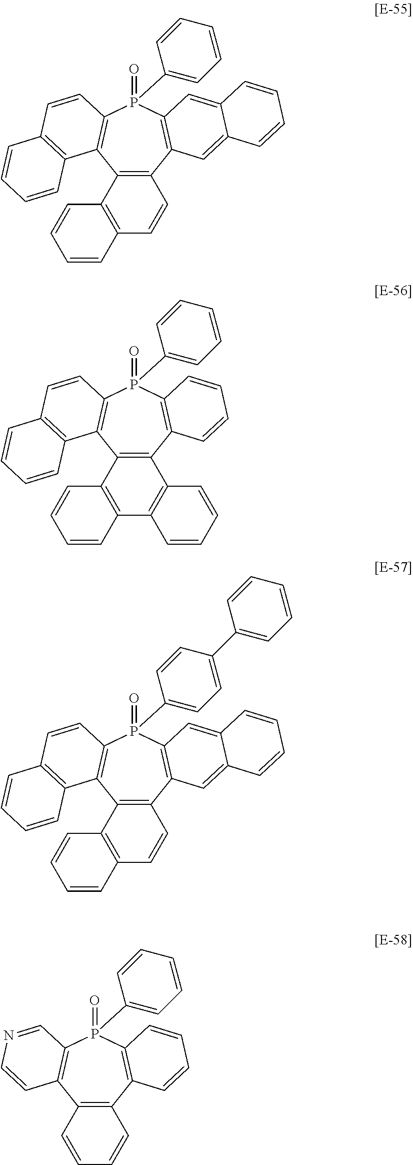 [SMILES: O=P1(c2ccccc2)c2cc3ccccc3cc2-c2ccc3ccccc3c2-c2c1ccc1ccccc21 ; O=P1(c2ccccc2)c2ccccc2-c2c(c3ccccc3c3ccccc23)-c2c1ccc1ccccc21 ; O=P1(c2ccc(-c3ccccc3)cc2)c2cc3ccccc3cc2-c2ccc3ccccc3c2-c2c1ccc1ccccc21 ; O=P1(c2ccccc2)c2ccccc2-c2ccccc2-c2ccncc21]

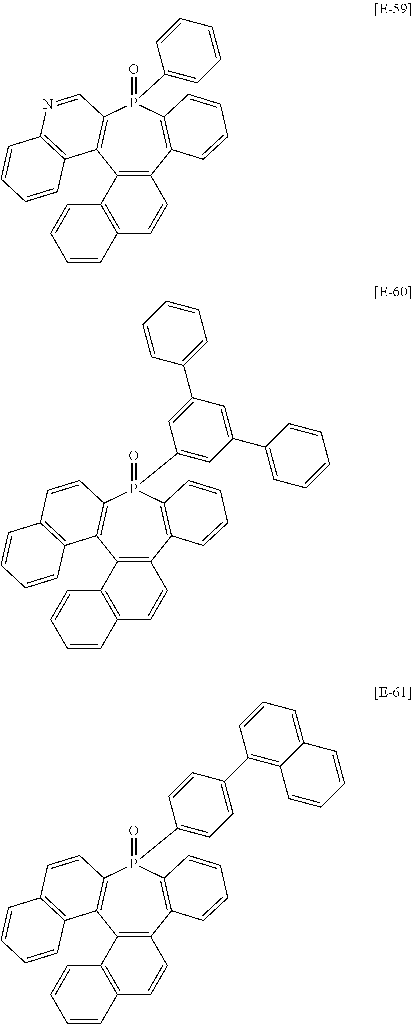 [SMILES: O=P1(c2ccccc2)c2ccccc2-c2ccc3ccccc3c2-c2c1cnc1ccccc21 ; O=P1(c2cc(-c3ccccc3)cc(-c3ccccc3)c2)c2ccccc2-c2ccc3ccccc3c2-c2c1ccc1ccccc21 ; O=P1(c2ccc(-c3cccc4ccccc34)cc2)c2ccccc2-c2ccc3ccccc3c2-c2c1ccc1ccccc21]

In addition, the electron transport layer may include the phosphine oxide compound alone or as a mixture with a dopant.

The dopant may be an n-type dopant that is used in a trace amount in order to make electron extraction from a cathode easy. The dopant may be an alkali metal, an alkali metal compound, an alkaline-earth metal, or an alkaline-earth metal compound.

For example, it may be an organometallic compound represented by Chemical Formula c.

Yₘ-M-(OA)ₙ [Chemical Formula c]

In Chemical Formula c,

Y includes a moiety consisting a single bond by a direct bond between one of C, N, O, and S, and M and a moiety consisting of a coordination bond between one of C, N, O, and S, and M and is a ligand chelated by the single bond and the coordination bond,

M is an alkali metal, an alkali earth metal, aluminum (Al), or a boron (B) atom, OA is a monovalent ligand capable of single-bonding or coordination-bonding with M,

O is oxygen.

A is one of a substituted or unsubstituted C1 to C30 alkyl group, a substituted or unsubstituted C5 to C50 aryl group, a substituted or unsubstituted C2 to C30 alkenyl group, a substituted or unsubstituted C2 to C20 alkynyl group, a substituted or unsubstituted C3 to C30 cycloalkyl group, a substituted or unsubstituted C5 to C30 cycloalkenyl group, and a substituted or unsubstituted C2 to C50 heteroaryl group having O, N, or S as a heteroatom,

when M is one metal selected from the alkali metal, m=1 and n=0,

when M is one metal selected from the alkali earth metal, m=1 and n=1 or m=2 and n=0,

when M is boron or aluminum, m is an integer ranging from 1 to 3 and n is an integer of 0 to 2, and m+n=3, and

‘substituted’ in the ‘substituted or unsubstituted’ refers to substitution with one or more substituent selected from deuterium, a cyano group, a halogen, a hydroxyl group, a nitro group, alkyl group, an alkoxy group, an alkylamino group, an arylamino group, a heteroarylamino group, an alkylsilyl group, an arylsilyl group, an aryloxy group, an aryl group, a heteroaryl group, germanium, phosphorus, and boron.

In the present invention, Y may independently be the same or different and may independently be selected from Chemical Formula c1 to Chemical Formula c39, but is not limited thereto.

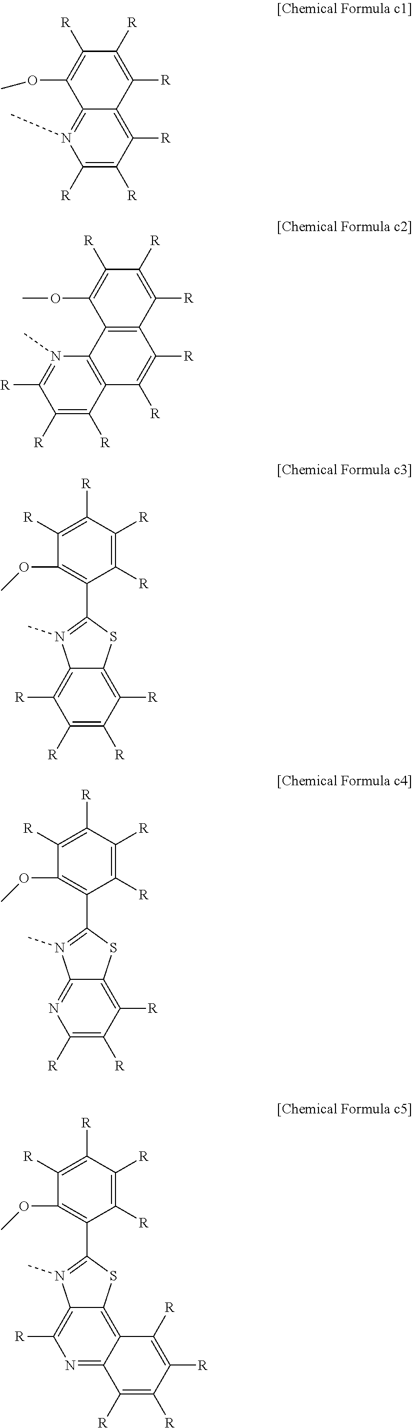

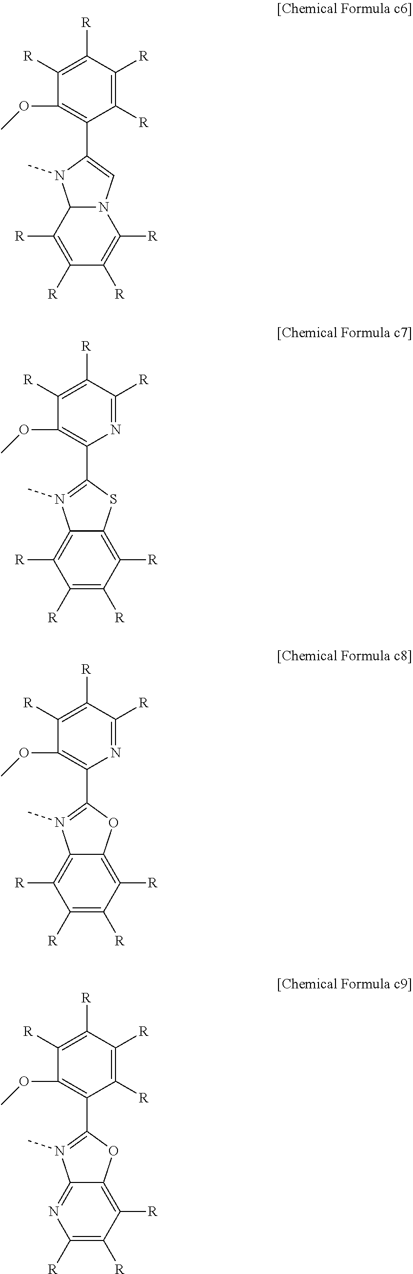

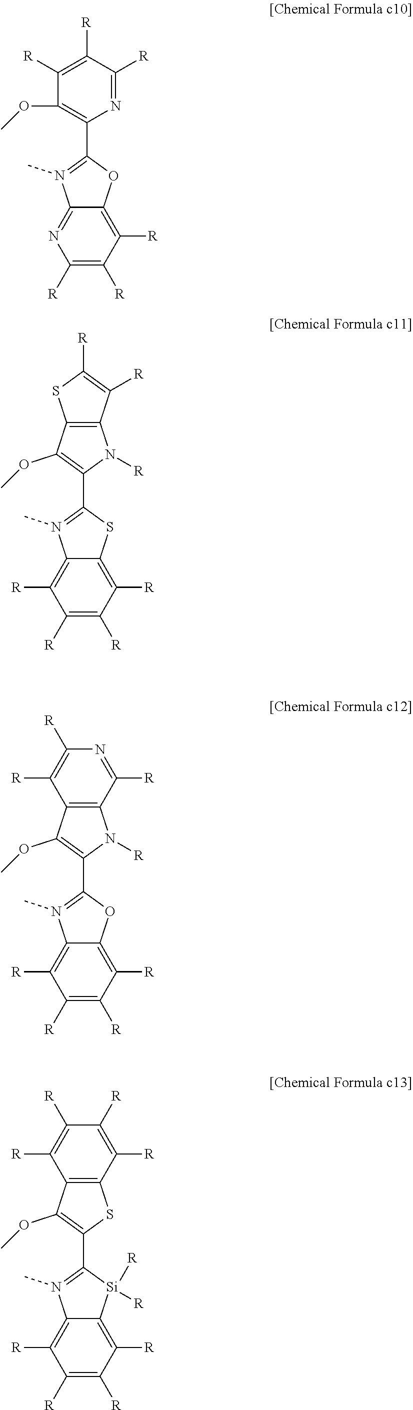

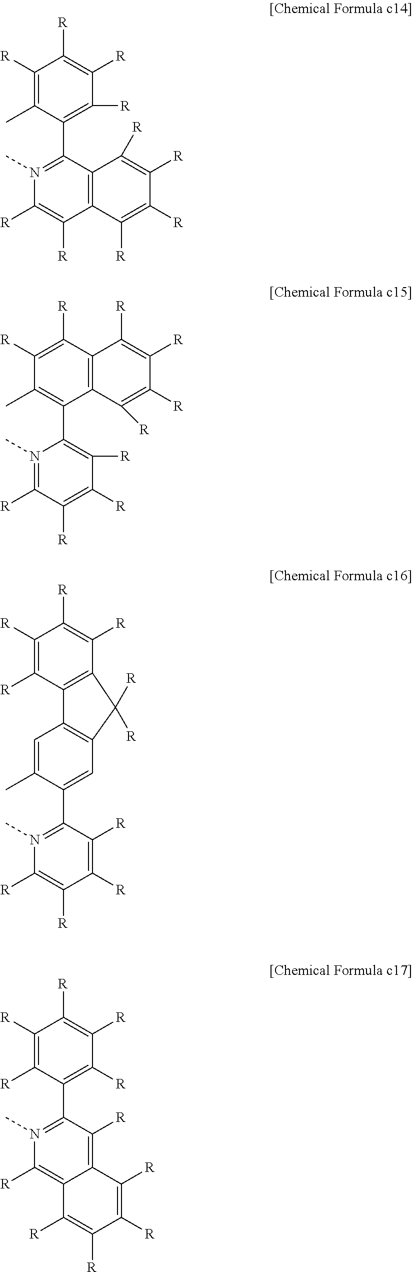

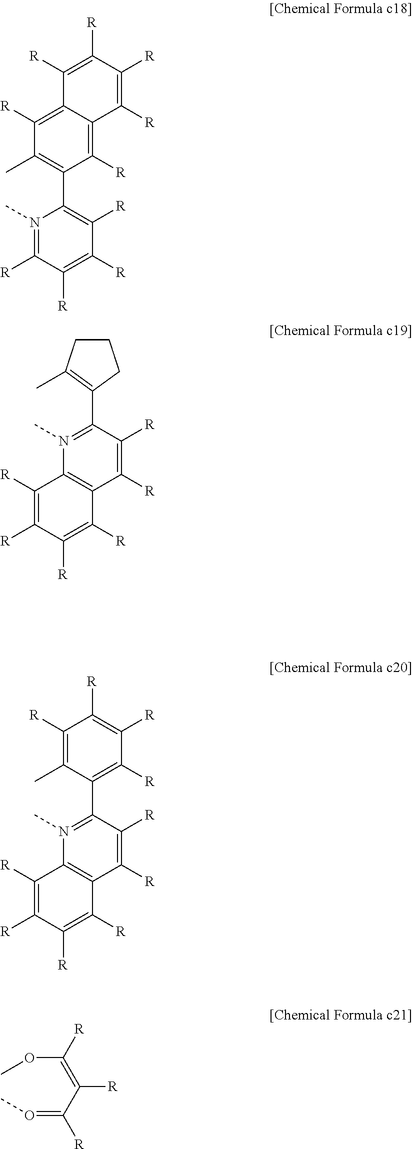

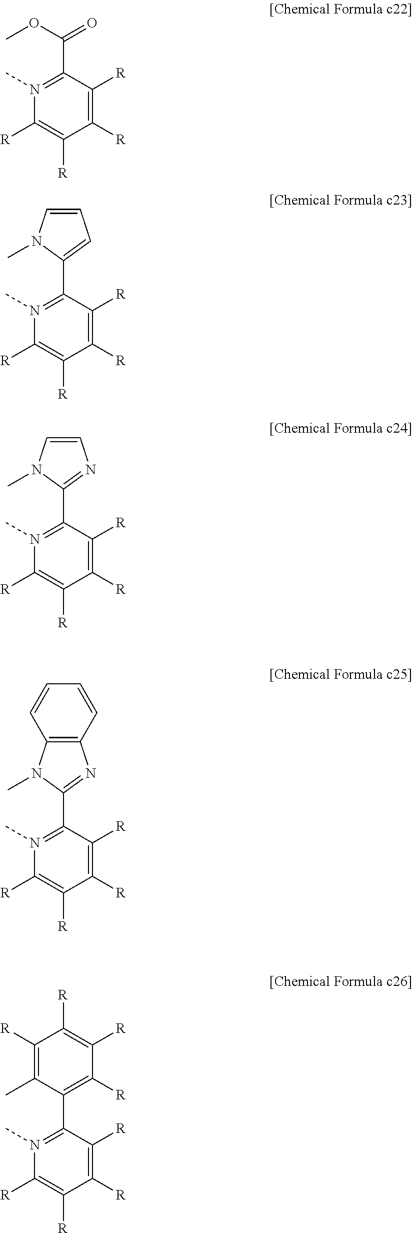

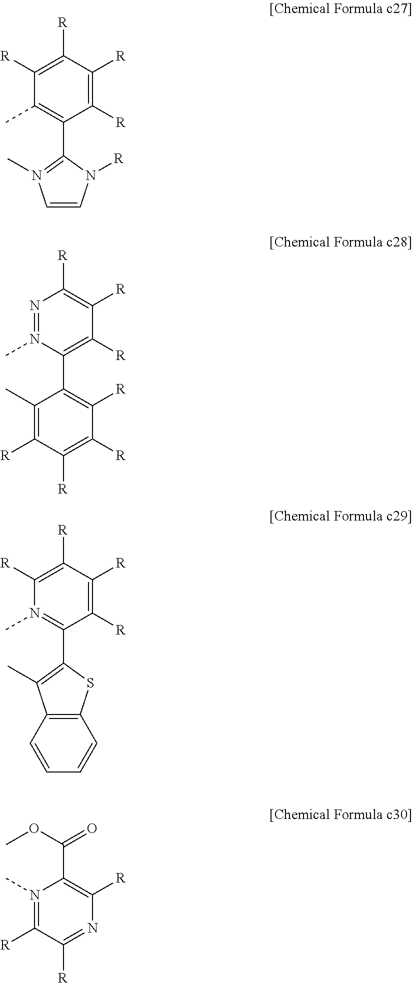

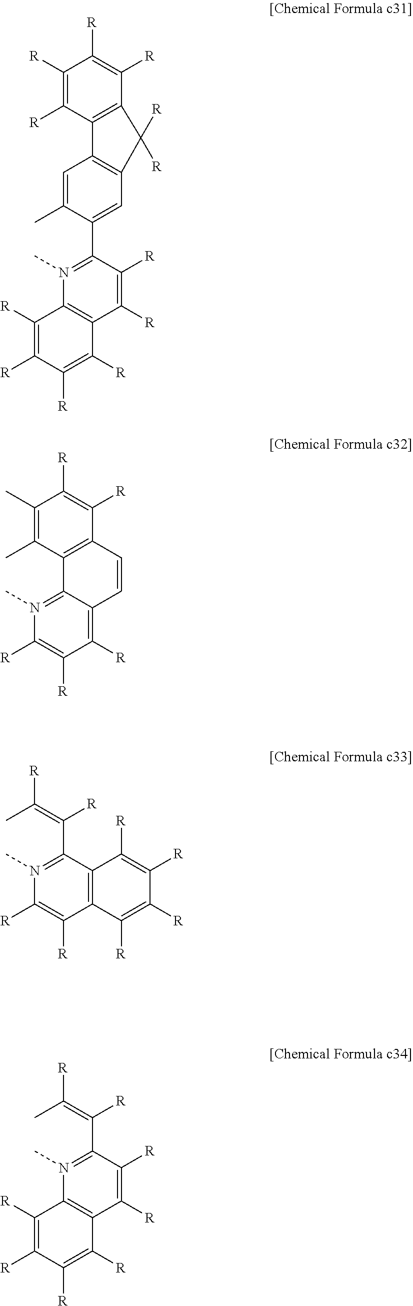

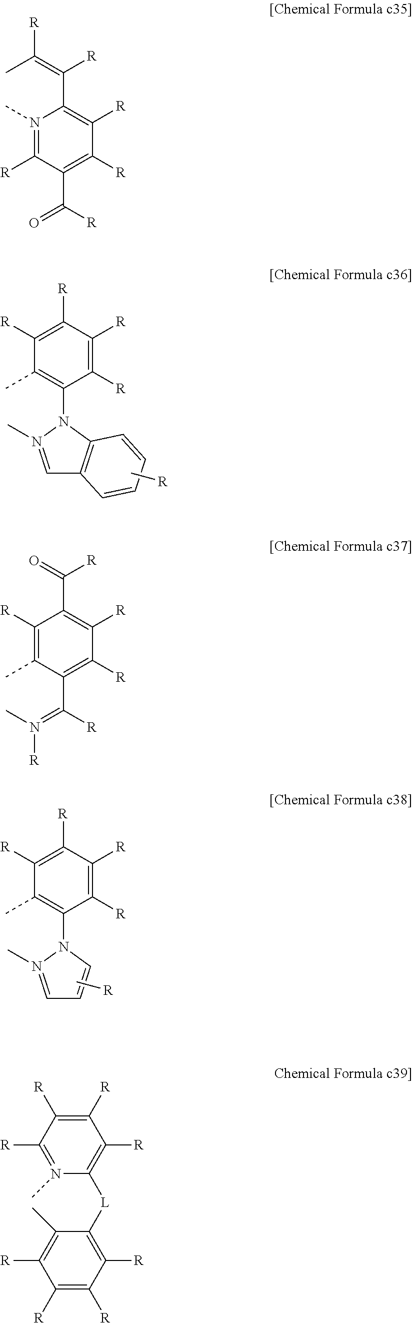

In Chemical Formula c1 to Chemical Formula c39,

R's are the same or different and are independently selected from hydrogen, deuterium, a halogen, a cyano group, a substituted or unsubstituted C1 to C30 alkyl group, a substituted or unsubstituted C6 to C30 aryl group, a substituted or unsubstituted C3 to C30 heteroaryl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C1 to C30 alkylamino group, a substituted or unsubstituted C1 to C30 alkylsilyl group, a substituted or unsubstituted C6 to C30 arylamino group, and a substituted or unsubstituted C6 to C30 arylsilyl group or is linked with an adjacent substitutent with alkylene or alkenylene to from a spiro ring or a fused ring.

In addition, referring to FIG. 2, the organic layer 105 may further include a hole auxiliary layer 150 between the anode 120 and the light emitting layer 130.

The hole auxiliary layer 150 may be at least one selected from a hole injection layer, a hole transport layer, and an electron blocking layer.

The anode 110 may be made of a conductor having a large work function to help hole injection, and may be for example made of a metal, a metal oxide, and/or a conductive polymer. The anode 110 may be for example a metal such as nickel, platinum, vanadium, chromium, copper, zinc, and gold or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; or a conductive polymer such as poly(3-methylthiophene), poly(3,4-(ethylene-1,2-dioxy)thiophene) (PEDT), polypyrrole, and polyaniline, but is not limited thereto.

The cathode 120 may be made of a conductor having a small work function to help electron injection, and may be for example made of a metal, a metal oxide and/or a conductive polymer. The cathode 120 may be for example a metal or an alloy thereof such as magnesium, calcium, sodium, potassium, titanium, indium, yttrium, lithium, gadolinium, aluminum silver, tin, lead, cesium, barium, and the like; a multi-layer structure material such as LiF/Al, LiO₂/Al, LiF/Ca, LiF/Al, and BaF₂/Ca, but is not limited thereto.

The organic optoelectronic device may be any device to convert electrical energy into photoenergy and vice versa without particular limitation, and may be, for example an organic photoelectric device, an organic light emitting diode, an organic solar cell, and an organic photo-conductor drum.

The organic light emitting diodes 100 and 200 may be manufactured by forming an anode or a cathode on a substrate, forming an organic layer using a dry film formation method such as a vacuum deposition method (evaporation), sputtering, plasma plating, and ion plating or a wet coating method such as spin coating, dipping, and flow coating, and forming a cathode or an anode thereon.

The organic light emitting diode may be applied to an organic light emitting diode display.

Hereinafter, the embodiments are illustrated in more detail with reference to examples. These examples, however, are not in any sense to be interpreted as limiting the scope of the invention.

Hereinafter, starting materials and reactants used in Examples and Synthesis Examples were purchased from Sigma-Aldrich Co., Ltd. or TCI Inc. as far as there in no particular comment or were synthesized by known methods.

### (Preparation of Compound for Organic Optoelectronic Device)

The compound as one specific examples of the present disclosure was synthesized through the following steps.

### Synthesis of First Compound for Organic Optoelectronic Device

### Synthesis Example 1: Synthesis of Compound A-1

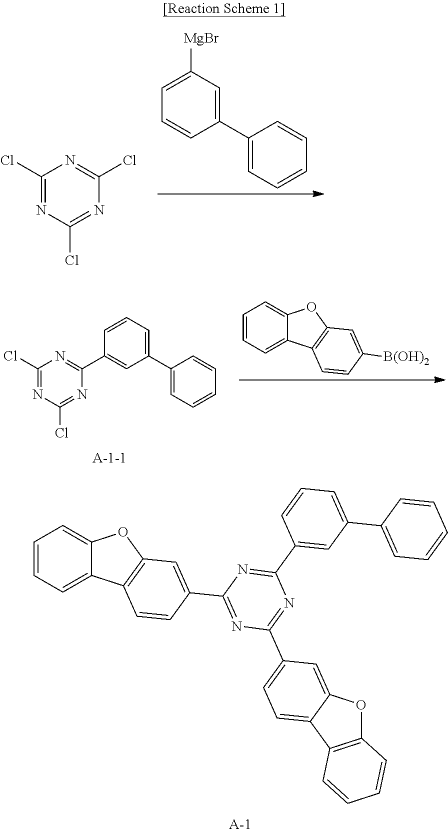

a) Synthesis of Intermediate A-1-1

15 g (81.34 mmol) of cyanuric chloride was dissolved in 200 mL of anhydrous tetrahydrofuran in a 500 mL round-bottomed flask, 1 equivalent of 3-biphenyl magnesium bromide solution (0.5 M tetrahydrofuran) was added thereto in a dropwise fashion at 0° C. under a nitrogen atmosphere, and the mixture was slowly heated up to room temperature. The reaction solution was stirred at room temperature for 1 hour and in 500 mL of ice water to separate layers. After separating an organic layer therefrom, the resultant was treated with anhydrous magnesium sulfate and concentrated. The concentrated residue was recrystallized with tetrahydrofuran and methanol to obtain 17.2 g of Intermediate A-1-1.

b) Synthesis of Compound A-1

17.2 g (56.9 mmol) of Intermediate A-1-1 were put in 200 mL of tetrahydrofuran and 100 mL of distilled water in a 500 mL round-bottomed flask, 2 equivalents of dibenzofuran-3-boronic acid (Cas: 395087-89-5), 0.03 equivalents of tetrakistriphenylphosphine palladium, and 2 equivalents of potassium carbonate were added thereto, and the mixture was heated and refluxed under a nitrogen atmosphere. After 18 hours, the reaction solution was cooled down, and a solid precipitated thereon was filtered and washed with 500 mL of water. The solid was recrystallized with 500 mL of monochlorobenzene to obtain 12.87 g of Compound A-1.

LC/MS calculated for: C39H23N3O2 Exact Mass: 565.1790 found for: 566.18 [M+H].

### Synthesis Example 2: Synthesis of Compound A-2

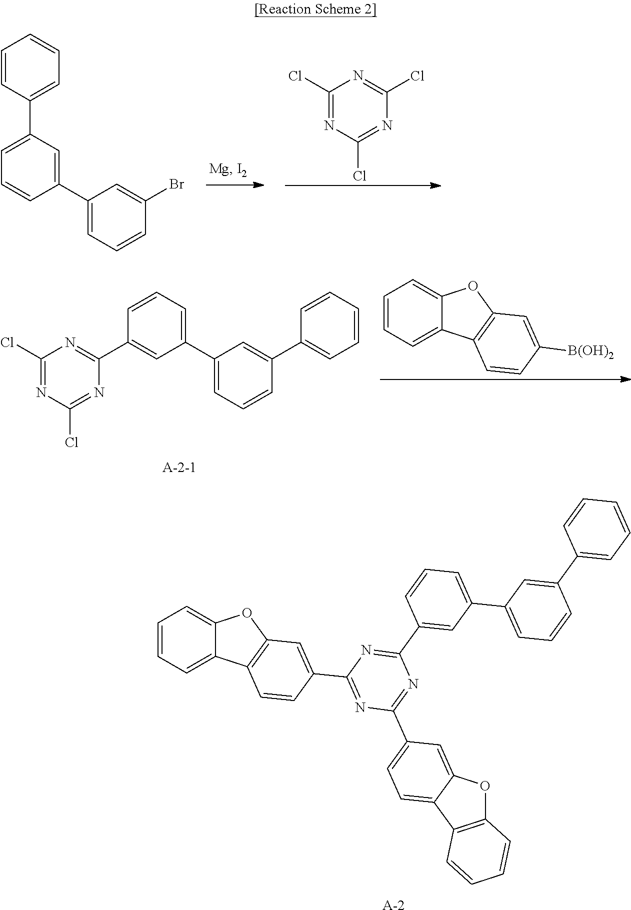

a) Synthesis of Intermediate A-2-1

7.86 g (323 mmol) of magnesium and 1.64 g (6.46 mmol) of iodine were put in 0.1 L of tetrahydrofuran (THF) under a nitrogen environment, the mixture was stirred for 30 minutes, and 100 g (323 mmol) of 3-bromo-tert-phenyl dissolved in 0.3 L of THF was slowly added thereto in a dropwise fashion at 0° C. over 30 minutes. The mixed solution was slowly added in a dropwise fashion to a solution prepared by dissolving 64.5 g (350 mmol) of cyanuric chloride in 0.5 L of THF at 0° C. for 30 minutes. When a reaction was complete, water was added to the reaction solution, and then, an extract was obtained by using dichloromethane (DCM), treated with anhydrous MgSO₄ to remove moisture, and then, filtered and concentrated under a reduced pressure. The obtained residue was separated and purified through flash column chromatography to obtain Intermediate A-2-1 (85.5 g, 70%).

b) Synthesis of Compound A-2

Compound A-2 was synthesized using Intermediate A-2-1 according to the same method as (b) of Synthesis Example 1.

LC/MS calculated for: C45H27N3O2 Exact Mass: 641.2103 found for 642.21 [M+H].

### Synthesis Example 3: Synthesis of Compound A-5

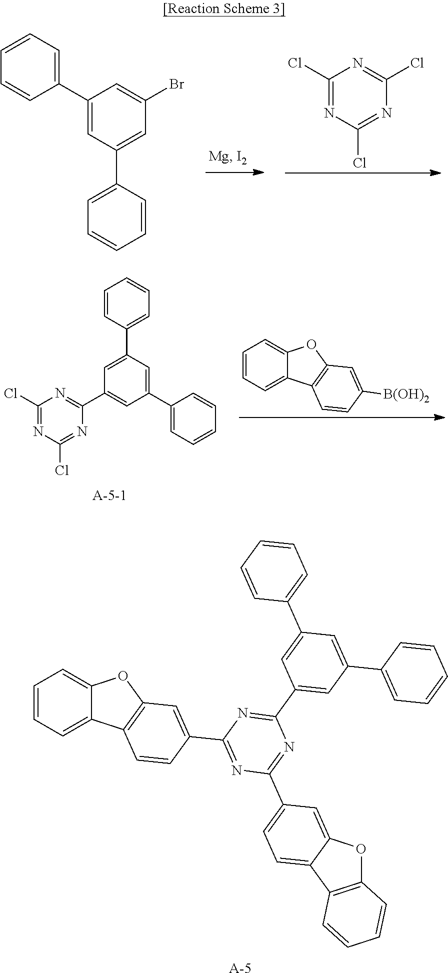

a) Synthesis of Intermediate A-5-1

7.86 g (323 mmol) of magnesium and 1.64 g (6.46 mmol) of iodine were put in 0.1 L of tetrahydrofuran (THF) under a nitrogen environment, the mixture was stirred for 30 minutes, and 100 g (323 mmol) of 1-bromo-3,5-diphenylbenzene dissolved in 0.3 L of THF was slowly added thereto in a dropwise fashion at 0° C. over 30 minutes. This obtained mixed solution was slowly added in a dropwise fashion to a solution prepared by dissolving 64.5 g (350 mmol) of cyanuric chloride in 0.5 L of THF at 0° C. over 30 minutes. When a reaction was complete, water was added to the reaction solution, and an extract was obtained by using dichloromethane (DCM), treated with anhydrous MgSO₄ to remove moisture, and then, filtered and concentrated under a reduced pressure. This obtained residue was separated and purified through flash column chromatography to obtain Intermediate A-5-1 (79.4 g, 65%).

b) Synthesis of Compound A-5

Compound A-5 was synthesized using Intermediate A-5-1 according to the same method as (b) of Synthesis Example 1.

LC/MS calculated for: C45H27N3O2 Exact Mass: 641.2103 found for 642.21 [M+H].

### Synthesis Example 4: Synthesis of Compound A-6

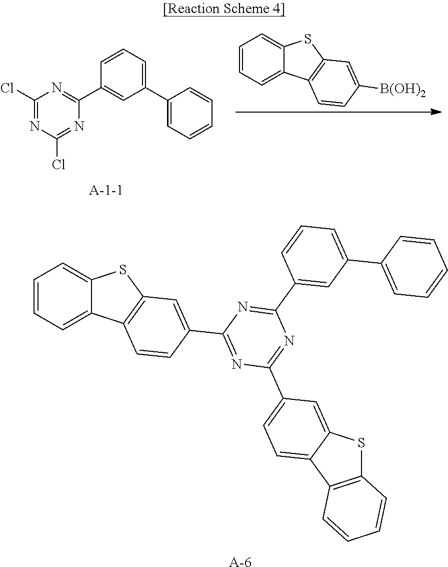

a) Synthesis of Compound A-6

Compound A-6 was synthesized according to the same method as (b) of Synthesis Example 1 by using dibenzothiophene-3-boronic acid (Cas No.: 108847-24-1) instead of Intermediate A-1-1 and dibenzofuran-3-boronic acid (Cas No.: 395087-89-5).

LC/MS calculated for: C39H23N3S2 Exact Mass: 597.1333 found for 598.13 [M+H].

### Synthesis Example 5: Synthesis of Compound A-15

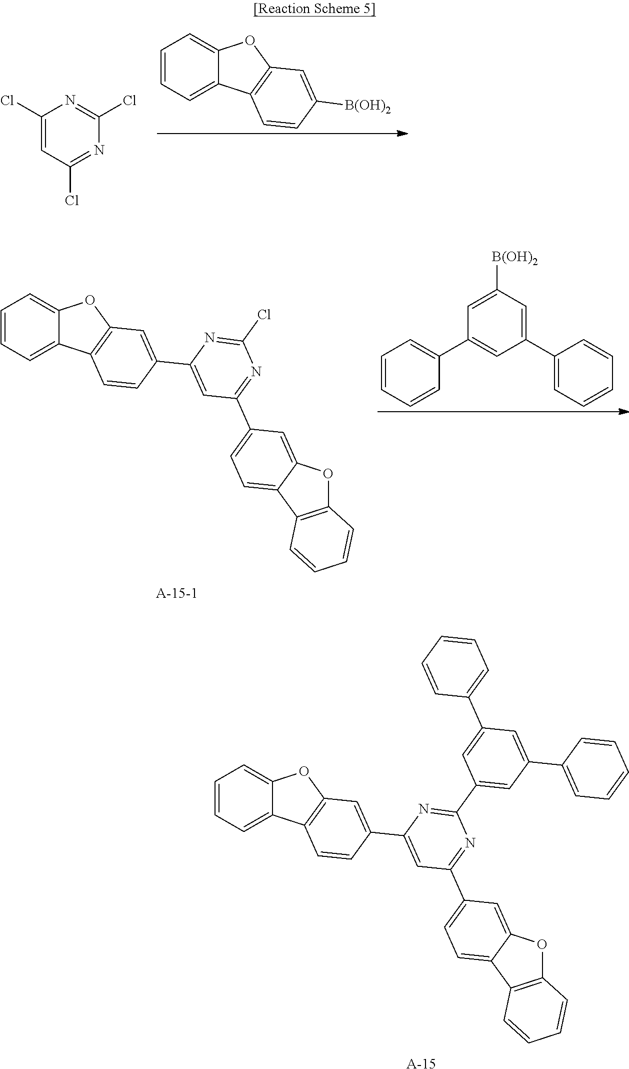

a) Synthesis of Intermediate A-15-1

18.3 g (100 mmol) of 2,4,6-trichloropyrimidine was put in 200 mL of tetrahydrofuran and 100 mL of distilled water in a 500 mL round-bottomed flask. 1.9 equivalents of dibenzofuran-3-boronic acid (Cas No.: 395087-89-5), 0.03 equivalents of tetrakistriphenylphosphine palladium, and 2 equivalents of potassium carbonate were added thereto, and the mixture was heated and refluxed under a nitrogen atmosphere. After 18 hours, the reaction solution was cooled down, and a solid precipitated therein was filtered and washed with 500 mL of water. The solid was recrystallized with 500 mL of monochlorobenzene to obtain 26.8 g of Intermediate A-15-1 (yield of 60%).

b) Synthesis of Compound A-15

Compound A-15 was synthesized according to the same method as (b) of Synthesis Example 1 by using Intermediate A-15-1 and 1.1 equivalents of 3,5-diphenylbenzeneboronic acid.

LC/MS calculated for: C46H28N2O2 Exact Mass: 640.2151 found for 641.21 [M+H].

### Synthesis Example 6: Synthesis of Compound A-21

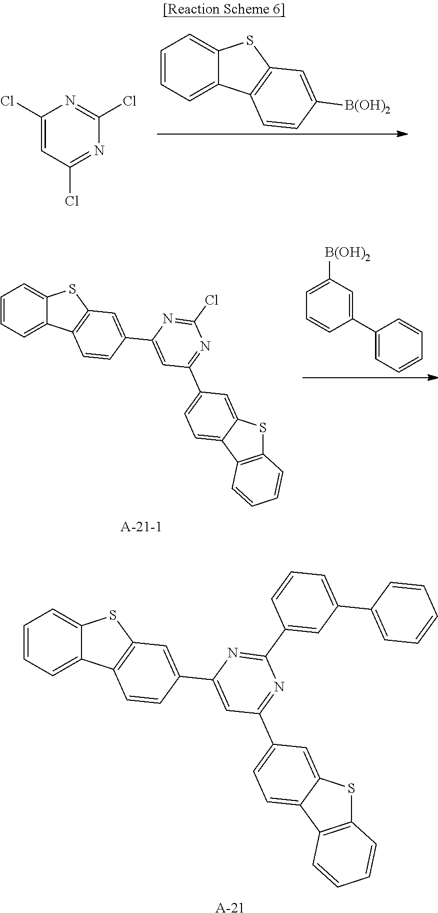

a) Synthesis of Intermediate A-21-1

Intermediate A-21-1 was synthesized according to the same method as (a) of Synthesis Example 5 by using dibenzothiophene-3-boronic acid (Cas No. 108847-24-1) instead of dibenzofuran-3-boronic acid (Cas: 395087-89-5).

b) Synthesis of Compound A-21

Compound A-21 was synthesized according to the same method as (b) of Synthesis Example 5 by using Intermediate A-21-1 and 1.1 equivalents of biphenyl-3-boronic acid.

LC/MS calculated for: C40H24N2S2 Exact Mass: 596.1381 found for 597.14 [M+H].

### Synthesis of Second Compound for Organic Optoelectronic Device

### Synthesis Example 7: Synthesis of Compound B-71

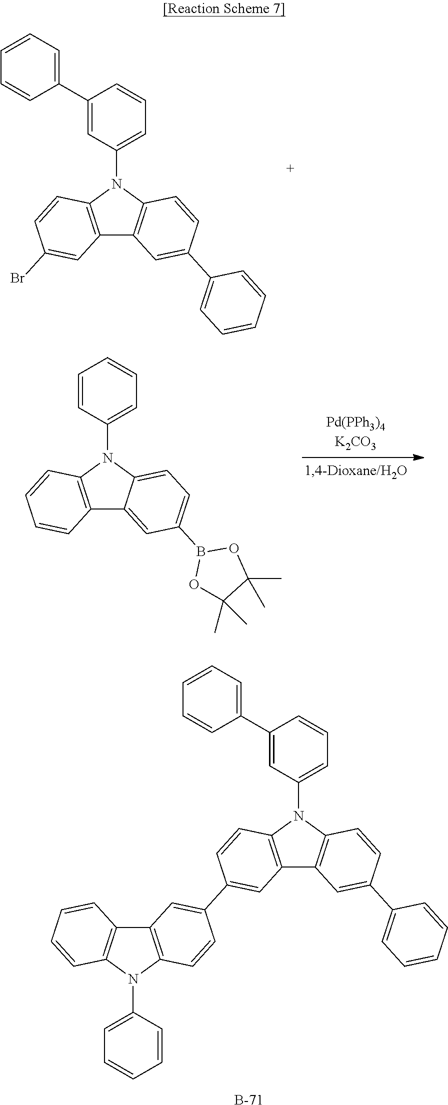

20.00 g (42.16 mmol) of 3-bromo-6-phenyl-N-metabiphenylcarbazole, 17.12 g (46.38 mmol) of N-phenylcarbazole-3-boronic ester, 175 mL of tetrahydrofuran and toluene (1:1), and 75 mL of a 2 M-potassium carbonate aqueous solution were mixed in a 500 mL round-bottomed flask under a nitrogen atmosphere equipped with an agitator, 1.46 g (1.26 mmol) of tetrakistriphenylphosphine palladium (0) was added thereto, and the mixture was heated and refluxed under a nitrogen flow for 12 hours. When a reaction was complete, the reactants were poured into methanol, and a solid therein was filtered and then, sufficiently washed with water and methanol and dried. A resulting material obtained therefrom was heated with and dissolved in 700 mL of chlorobenzene, the solution was silica gel-filtered, and a solid obtained by completely removing a solvent was heated with and dissolved in 400 mL of chlorobenzene and then, recrystallized to obtain 18.52 g of Compound B-71 (yield of 69%).

calcd. C₄₂H₃₂N₂; C, 90.54; H, 5.07; N, 4.40. found: C, 90.54; H, 5.07; N, 4.40.

### Synthesis Example 8: Synthesis of Compound B-78

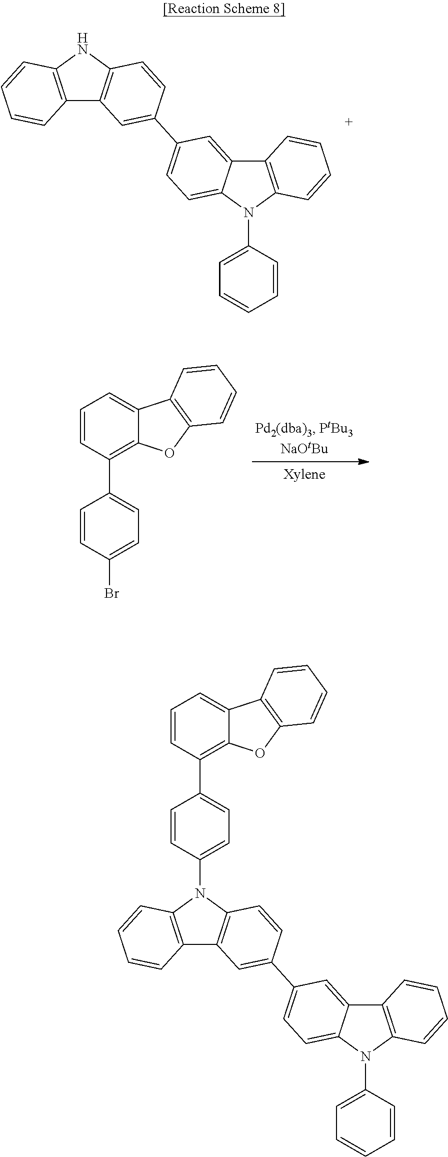 [SMILES: c1ccc(-n2c3ccccc3c3cc(-c4ccc5[nH]c6ccccc6c5c4)ccc32)cc1 ; Brc1ccc(-c2cccc3c2oc2ccccc23)cc1 ; c1ccc(-n2c3ccccc3c3cc(-c4ccc5c(c4)c4ccccc4n5-c4ccc(-c5cccc6c5oc5ccccc56)cc4)ccc32)cc1]

6.3 g (15.4 mmol) of N-phenyl-3,3-bicarbazole, 5.0 g (15.4 mmol) of 4-(4-bromophenyl)dibenzo[b,d]furan, 3.0 g (30.7 mmol) of sodium t-butoxide, 0.9 g (1.5 mmol) of tris(dibenzylideneacetone)dipalladium, and 1.2 mL of tri t-butylphosphine (50% in toluene) were mixed with 100 mL of xylene in a 250 mL round flask, and the mixture was heated and refluxed under a nitrogen flow for 15 hours. The obtained mixture was added to 300 mL of methanol, and a solid crystallized therein was filtered, dissolved in dichlorobenzene, filtered with silica gel/Celite, and after removing an appropriate amount of an organic solvent, recrystallized with methanol to obtain Compound B-78 (7.3 g, a yield of 73%).

calcd. C48H30N2O; C, 88.59; H, 4.65; N, 4.30; 0, 2.46. found: C, 88.56; H, 4.62; N, 4.20; 0, 2.43.

### Synthesis of Third Compound for Organic Optoelectronic Device

### Synthesis Example 9: Synthesis of Compound E-7

13.5 g of Compound E-7 was obtained referring to the synthesis method of B15 compound on page 76 of International Publication WO2016-162440.

LC/MS calculated for: C38H27O1P1 Exact Mass: 530.1800 found for: 531.18 [M+H].

### Synthesis Example 10: Synthesis of Compound E-39

8.3 g of Compound E-39 was obtained referring to the synthesis method of “Synthesis of Structure 34” of paragraph 176 of US Publication No. 2014-0332790.

LC/MS calculated for: C39H26N1O1P1 Exact Mass: 555.1752 found for: 556.18 [M+H].

### Synthesis Example 11: Synthesis of Compound E-53

5.7 g of Compound E-53 was obtained referring to the synthesis method of paragraph 101 of Korean Publication KR2016-0102528.

LC/MS calculated for: C32H21O1P1 Exact Mass: 452.1330 found for: 453.13 [M+H].

### Comparative Synthesis Example 1: Synthesis of Comparative Compound 6 (Comp-6)

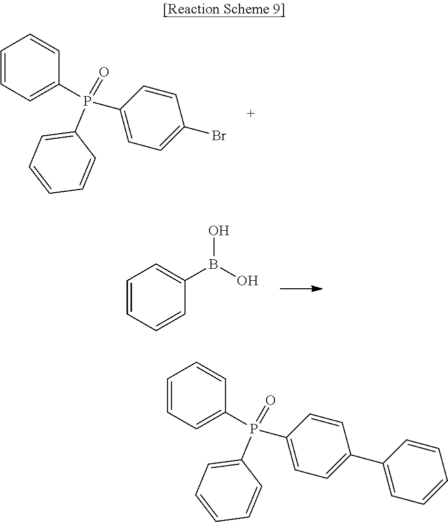 [SMILES: O=P(c1ccccc1)(c1ccccc1)c1ccc(Br)cc1 ; OB(O)c1ccccc1 ; O=P(c1ccccc1)(c1ccccc1)c1ccc(-c2ccccc2)cc1]

20.00 g (56.00 mmol) of (4-bromophenyl)diphenylphosphine oxide, 7.51 g (61.59 mmol) of phenyl boronic acid, and 250 mL of tetrahydrofuran:toluene (1:1), and 100 mL of 2 M-potassium carbonate aqueous solution were mixed in a 500 mL round-bottomed flask equipped with an agitator under a nitrogen atmosphere, 3.00 g (2.59 mmol) of tetrakistriphenylphosphine palladium (0) was added thereto, and the mixture was heated and refluxed under a nitrogen flow for 12 hours. When a reaction was complete, the reactants were poured into methanol, and a solid therein was filtered and then, sufficiently washed with water and methanol and dried. A resulting material obtained therefrom was heated with and dissolved in 700 mL of chlorobenzene, the solution was silica gel-filtered, and a solid obtained by completely removing a solvent was heated with and dissolved in 400 mL of chlorobenzene and then, recrystallized to obtain 14.88 g (yield of 75%) of Comparative Compound 6.

LC/MS calculated for: C2411190P Exact Mass: 354.1220 found for: 354.12 [M+H].

### Manufacture of Organic Light Emitting Diode

### Example 1

A glass substrate was coated with ITO (indium tin oxide) to be 1500 Å thick and then, ultrasonic wave-washed with a distilled water. After washing with distilled water, the glass substrate was ultrasonic wave-washed with a solvent such as isopropyl alcohol, acetone, methanol and the like, dried, moved to a plasma-cleaner, and then, cleaned with oxygen plasma for 10 minutes and moved to a vacuum depositor. The obtained ITO transparent electrode was used as an anode, a 700 Å-thick hole injection layer was formed on the ITO substrate by vacuum-depositing Compound A, and a hole transport layer was formed by depositing Compound B on the injection layer with a 50 Å thickness and then Compound C with a 1020 Å thickness. On the hole transport layer, a 400 Å-thick light emitting layer was formed by vacuum-depositing Compound A-5 of Synthesis Example 3 and Compound B-40 as hosts simultaneously and 10 wt % of tris(2-phenylpyridine)iridium(III) [Ir(ppy)₃] as a dopant. Herein Compound A-5 and Compound B-40 were used in a weight ratio of 25:75 and each ratio of Examples was separately described. Subsequently, on the light emitting layer, Compound E-7 of Synthesis Example 9 and Liq were simultaneously vacuum-deposited in a 1:1 ratio to form a 300 Å-thick electron transport layer, and on the electron transport layer, 15 Å-thick Liq and 1200 Å-thick Al were sequentially vacuum-deposited to form a cathode, manufacturing an organic light emitting diode.

The organic light emitting diode had a structure of having five organic thin layers, specifically a structure of ITO/Compound A (700 Å)/Compound B (50 Å)/Compound C (1020 Å)/EML[Compound A-5:B-40:Ir(ppy)₃=22.5 wt %:67.5 wt %:10 wt %] (400 Å)/Compound E-7:Liq (300 Å)/Liq (15 Å)/Al (1200 Å).

Compound A: N4,N4′-diphenyl-N4,N4′-bis(9-phenyl-9-carbazol-3-yl)biphenyl-4,4′-diamine

Compound B: 1,4,5,8,9,11-hexaazatriphenylene-hexacarbonitrile (HAT-CN),

Compound C: N-(biphenyl-4-yl)-9,9-dimethyl-N-(4-(9-phenyl-9H-carbazol-3-yl)phenyl)-9H-fluoren-2-amine

### Examples 2 to 30

Each device of Examples 2 to 30 was manufactured according to the same method as Example 1 by using hosts and ETL as shown in Table 1.

### Reference Examples 1 to 20

Each device of Reference Examples 1 to 20 was manufactured according to the same method as Example 1 by using hosts in Table 1 and Alq3 (aluminum quinolate) or Comparative Compound 6 as ETL.

### Evaluation 1: Syngeneic Effect of Luminous Efficiency and Life-Span

Luminous efficiency and life-span characteristics of the organic light emitting diodes according to Examples 1 to 30 and Reference Examples 1 to 20 were evaluated. Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and, the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

The luminance, current density, and voltage obtained from the (1) and (2) were used to calculate power efficiency (lm/W) at the same current density (10 mA/cm²). Efficiency was indicated as relative values based on 100% of Reference Example 1.

(4) Measurement of Life-Span

T97 life-spans of the organic light emitting diodes according to Examples 1 to 30 and Reference Examples 1 to 20 were measured as a time when their luminance decreased down to 97% relative to the initial luminance (cd/m²) after emitting light with 18000 cd/m² as the initial luminance (cd/m²) and measuring their luminance decrease depending on a time with a Polanonix life-span measurement system. Life-spans were indicated as relative values based on 100% of Reference Example 1.

TABLE 1

| | | Host | | | | |
|---|---|---|---|---|---|---|
| | | mixing | | | | T97 |
| | | ratio of | | | | (@18000 |
| | | EML | | | lm/W | nit, |
| | | (weight | | Vd | (relative | relative |
| | EML | ratio) | ETL | (V) | value) | value) |
| Reference | A-5:B-40 | 25:75 | AlQ3 | 5.41 | 100% | 100% |
| Example 1 | | | | | | |
| Example 1 | A-5:B-40 | 25:75 | E-7 | 4.48 | 137% | 1000% |
| Example 2 | A-5:B-40 | 25:75 | E-39 | 4.58 | 129% | 522% |
| Example 3 | A-5:B-40 | 25:75 | E-53 | 4.49 | 130% | 544% |
| Reference | A-5:B-40 | 25:75 | Comp-6 | 5.24 | 93% | 50% |
| Example 2 | | | | | | |
| Reference | A-5:B-14 | 30:70 | AlQ3 | 5.00 | 101% | 119% |
| Example 3 | | | | | | |
| Example 4 | A-5:B-14 | 30:70 | E-7 | 4.49 | 130% | 912% |
| Example 5 | A-5:B-14 | 30:70 | E-39 | 4.54 | 124% | 482% |
| Example 6 | A-5:B-14 | 30:70 | E-53 | 4.50 | 124% | 502% |
| Reference | A-5:B-14 | 30:70 | Comp-6 | 5.31 | 72% | 53% |
| Example 4 | | | | | | |
| Reference | A-1:B-71 | 30:70 | AlQ3 | 4.87 | 100% | 91% |
| Example 5 | | | | | | |
| Example 7 | A-1:B-71 | 30:70 | E-7 | 4.43 | 130% | 909% |
| Example 8 | A-1:B-71 | 30:70 | E-39 | 4.48 | 123% | 479% |
| Example 9 | A-1:B-71 | 30:70 | E-53 | 4.43 | 124% | 499% |
| Reference | A-1:B-71 | 30:70 | Comp-6 | 5.11 | 68% | 72% |
| Example 6 | | | | | | |
| Reference | A-2:B-71 | 30:70 | AlQ3 | 4.77 | 102% | 105% |
| Example 7 | | | | | | |
| Example 10 | A-2:B-71 | 30:70 | E-7 | 4.37 | 130% | 911% |
| Example 11 | A-2:B-71 | 30:70 | E-39 | 4.42 | 124% | 481% |
| Example 12 | A-2:B-71 | 30:70 | E-53 | 4.38 | 124% | 501% |
| Reference | A-2:B-71 | 30:70 | Comp-6 | 5.10 | 101% | 13% |
| Example 8 | | | | | | |
| Reference | A-2:B-40 | 30:70 | AlQ3 | 4.58 | 98% | 119% |
| Example 9 | | | | | | |
| Example 13 | A-2:B-40 | 30:70 | E-7 | 4.28 | 130% | 912% |
| Example 14 | A-2:B-40 | 30:70 | E-39 | 4.33 | 123% | 482% |
| Example 15 | A-2:B-40 | 30:70 | E-53 | 4.28 | 124% | 502% |
| Reference | A-2:B-40 | 30:70 | Comp-6 | 4.78 | 66% | 44% |
| Example 10 | | | | | | |
| Reference | A-2:B-14 | 30:70 | AlQ3 | 4.76 | 100% | 129% |
| Example 11 | | | | | | |
| Example 16 | A-2:B-14 | 30:70 | E-7 | 4.37 | 130% | 913% |
| Example 17 | A-2:B-14 | 30:70 | E-39 | 4.42 | 123% | 483% |
| Example 18 | A-2:B-14 | 30:70 | E-53 | 4.37 | 124% | 503% |
| Reference | A-2:B-14 | 30:70 | Comp-6 | 5.01 | 78% | 40% |
| Example 12 | | | | | | |
| Reference | A-2:B-78 | 30:70 | AlQ3 | 5.06 | 98% | 113% |
| Example 13 | | | | | | |
| Example 19 | A-2:B-78 | 30:70 | E-7 | 4.52 | 130% | 911% |
| Example 20 | A-2:B-78 | 30:70 | E-39 | 4.57 | 123% | 481% |
| Example 21 | A-2:B-78 | 30:70 | E-53 | 4.53 | 124% | 501% |
| Reference | A-2:B-78 | 30:70 | Comp-6 | 5.10 | 99% | 42% |
| Example 14 | | | | | | |
| Reference | A-6:B-14 | 30:70 | A1Q3 | 4.70 | 96% | 95% |
| Example 15 | | | | | | |
| Example 22 | A-6:B-14 | 30:70 | E-7 | 4.34 | 129% | 910% |
| Example 23 | A-6:B-14 | 30:70 | E-39 | 4.39 | 123% | 480% |
| Example 24 | A-6:B-14 | 30:70 | E-53 | 4.34 | 123% | 500% |
| Reference | A-6:B-14 | 30:70 | Comp-6 | 4.99 | 43% | 27% |
| Example 16 | | | | | | |
| Reference | A-15:B-14 | 30:70 | AlQ3 | 5.17 | 102% | 68% |
| Example 17 | | | | | | |
| Example 25 | A-15:B-14 | 30:70 | E-7 | 4.57 | 130% | 907% |
| Example 26 | A-15:B-14 | 30:70 | E-39 | 4.62 | 124% | 477% |
| Example 27 | A-15:B-14 | 30:70 | E-53 | 4.58 | 124% | 497% |
| Reference | A-15:B-14 | 30:70 | Comp-6 | 5.10 | 22% | 59% |
| Example 18 | | | | | | |
| Reference | A-21:B-14 | 30:70 | AlQ3 | 5.09 | 104% | 75% |
| Example 19 | | | | | | |
| Example 28 | A-21:B-14 | 30:70 | E-7 | 4.53 | 131% | 908% |
| Example 29 | A-21:B-14 | 30:70 | E-39 | 4.58 | 124% | 478% |
| Example 30 | A-21:B-14 | 30:70 | E-53 | 4.54 | 125% | 498% |
| Reference | A-21:B-14 | 30:70 | Comp-6 | 5.17 | 89% | 55% |
| Example 20 | | | | | | |

Referring to results of Table 1, the organic light emitting diodes using a combination of specific hosts of the present invention combined with a specific electron transport layer material according to examples showed decrease of a driving voltage and increase of efficiency, particularly a life-span compared with generally-used Alq3. In addition, a driving voltage, efficiency, and life-span were improved particularly a life-span was remarkably improved compared with Reference Example using Comparative Compound 6 without a fused ring as electron transport layer material.

These effects were shown in case of a pyrimidine core as well as a triazine core. Therefore, from the device data, it was confirmed that when dibenzofuran or dibenzothiophene of the first host was linked with the ET core group directly, life-spans of the corresponding materials in a device were improved through effective LUMO expansion and fusion of rings.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

### DESCRIPTION OF SYMBOLS

100, 200: organic light emitting diode
105: organic layer
110: cathode
120: anode
130: light emitting layer
140: electron transport layer
150: hole auxiliary layer

1. An organic optoelectronic device, comprising
a cathode and an anode facing each other;
a light emitting layer between the cathode and the anode; and
an electron transport layer between the cathode and the light emitting layer,
wherein the light emitting layer includes at least one of a first compound for an organic optoelectronic device represented by Chemical Formula 1 and at least one of a second compound for an organic optoelectronic device represented by Chemical Formula 2, and
the electron transport layer includes at least one of a third compound for an organic optoelectronic device represented by Chemical Formula 3: 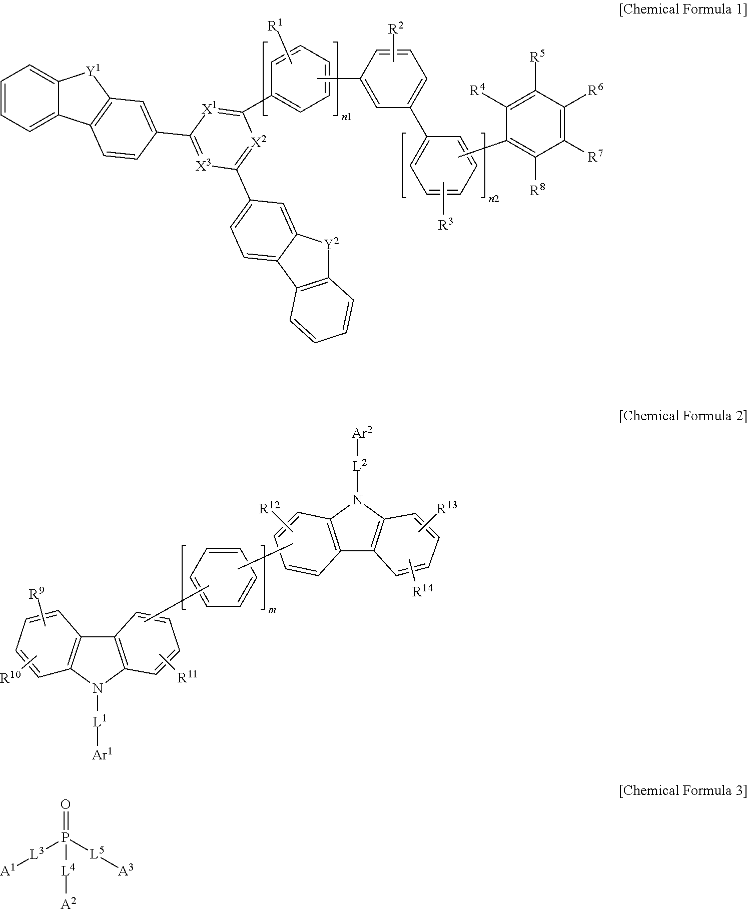
wherein, in Chemical Formula 1,
X¹ to X³ are independently N or CR^{a},
at least two of X¹ to X³ are N,
Y¹ and Y² are independently O or S,
n1 and n2 are independently an integer of 0 or 1, and
R^{a} and R¹ to R⁸ are independently hydrogen, deuterium, a cyano group, a nitro group, a substituted or unsubstituted C1 to C10 alkyl group, a substituted or unsubstituted C6 to C30 aryl group, a substituted or unsubstituted C2 to C30 heterocyclic group, or a combination thereof;
wherein, in Chemical Formula 2,
L¹ and L² are independently a single bond, a substituted or unsubstituted C6 to C30 arylene group, a substituted or unsubstituted C2 to C30 heteroarylene group, or a combination thereof,
Ar¹ and Ar² are independently a substituted or unsubstituted C6 to C30 aryl group, a substituted or unsubstituted carbazolyl group, a substituted or unsubstituted dibenzofuranyl group, a substituted or unsubstituted dibenzothiophenyl group, or a combination thereof,
R⁹ to R¹⁴ are independently hydrogen, deuterium, a substituted or unsubstituted C1 to C20 alkyl group, a substituted or unsubstituted C6 to C30 aryl group, a substituted or unsubstituted C2 to C30 heterocyclic group, or a combination thereof, and
m is an integer of 0 to 2;
wherein, in Chemical Formula 3,
L³ to L⁵ are independently a single bond, a substituted or unsubstituted C6 to C30 arylene group, a substituted or unsubstituted C2 to C30 heteroarylene group, or a combination thereof,
A¹ to A³ are independently a substituted or unsubstituted C6 to C30 aryl group, a substituted or unsubstituted C2 to C30 heterocyclic group, or a combination thereof,
A¹ to A³ are independently present or adjacent groups are linked with each other to form at least one of a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring,
when A¹ to A³ are independently present, at least one of A¹ to A³ is a substituted or unsubstituted fused aryl group or a substituted or unsubstituted fused heterocyclic group, and
“substituted” of Chemical Formulae 1 to 3 refers to replacement of at least one hydrogen by deuterium, a C1 to C4 alkyl group, a C6 to C18 aryl group, or a C2 to C30 heteroaryl group.

2. The organic optoelectronic device as claimed in claim 1, wherein Chemical Formula 1 is represented by Chemical Formula 1-I, Chemical Formula 1-II or Chemical Formula 1-III: 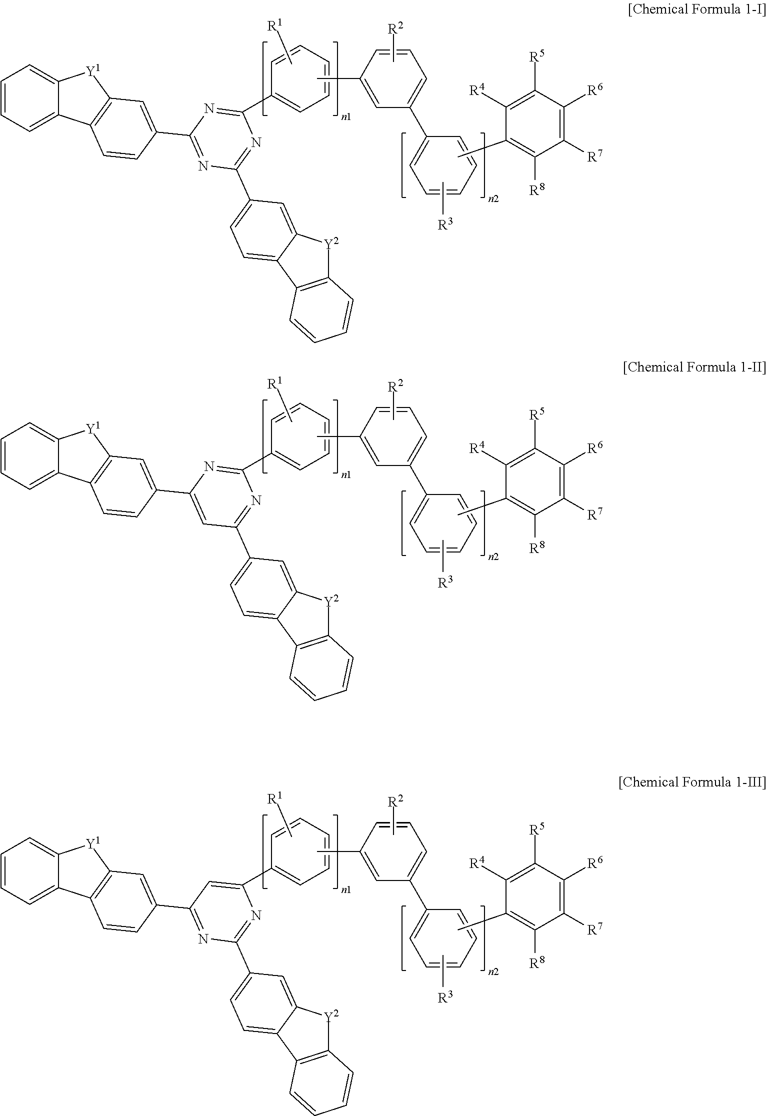
wherein, in Chemical Formula 1-I, Chemical Formula 1-II, and Chemical Formula 1-III,
Y¹ and Y² are independently O or S,
n1 and n2 are independently an integer of 0 or 1, and
R¹ to R⁸ are independently hydrogen, deuterium, a cyano group, a nitro group, a substituted or unsubstituted C1 to C10 alkyl group, a substituted or unsubstituted C6 to C30 aryl group, a substituted or unsubstituted C2 to C30 heterocyclic group, or a combination thereof.

3. The organic optoelectronic device as claimed in claim 1, wherein Chemical Formula 1 is represented by Chemical Formula 1A, Chemical Formula 1B or Chemical Formula 1C: 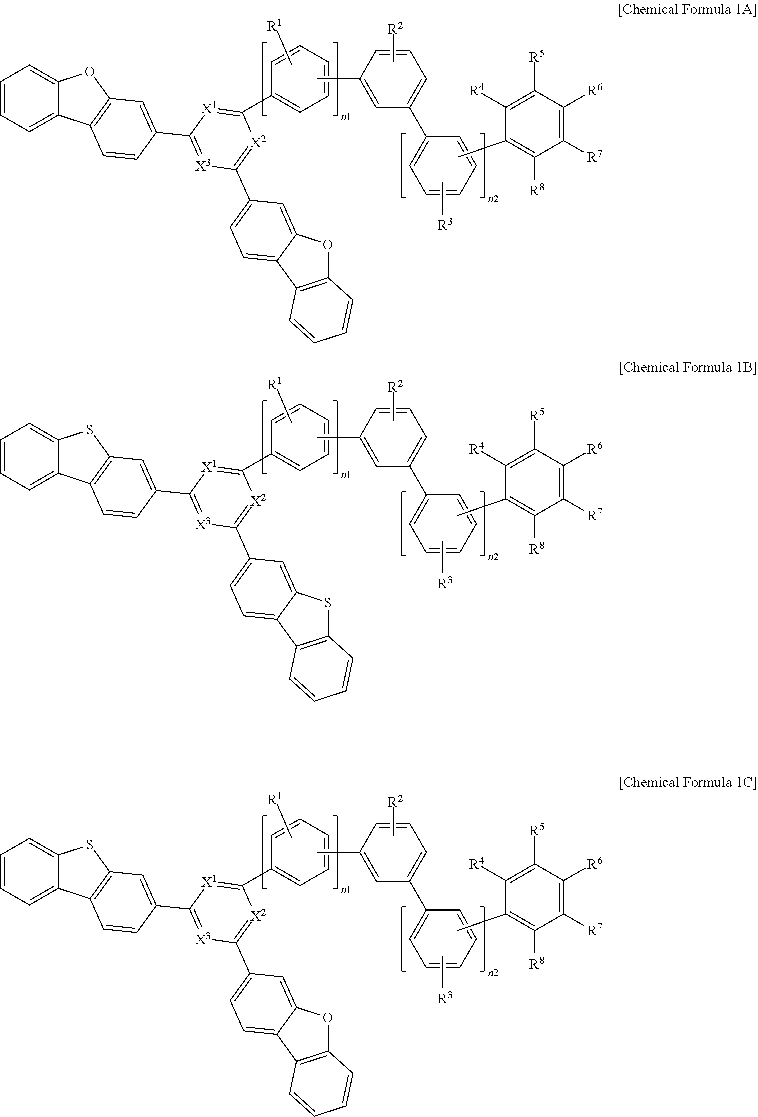
wherein, in Chemical Formula 1A, Chemical Formula 1B, and Chemical Formula 1C,
X¹ to X³ are independently N or CH,
at least two of X¹ to X³ are N,
n1 and n2 are independently an integer of 0 or 1, and
R¹ to R⁸ are independently hydrogen, deuterium, a cyano group, a nitro group, a substituted or unsubstituted C1 to C10 alkyl group, a substituted or unsubstituted C6 to C30 aryl group, a substituted or unsubstituted C2 to C30 heterocyclic group, or a combination thereof.

4. The organic optoelectronic device as claimed in claim 1, wherein Chemical Formula 1 is represented by Chemical Formula 1-1 or 1-2: 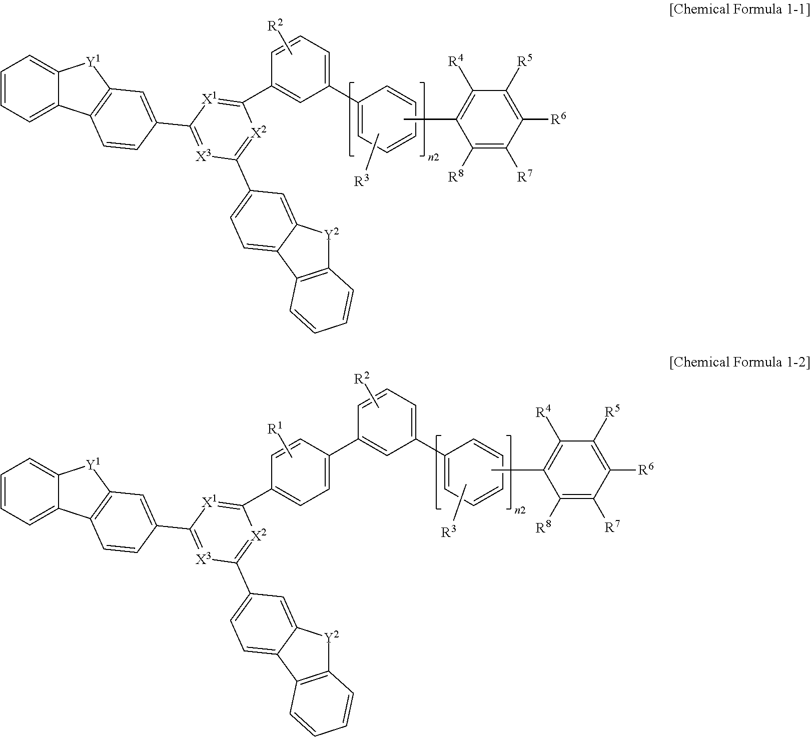
wherein, in Chemical Formulae 1-1 to 1-2,
X¹ to X³ are independently N or CH,
at least two of X¹ to X³ are N,
Y¹ and Y² are independently O or S,
n2 is an integer of 0 or 1, and
R¹ to R⁸ are independently hydrogen, deuterium, a cyano group, a nitro group, a substituted or unsubstituted C1 to C10 alkyl group, a substituted or unsubstituted C6 to C30 aryl group, a substituted or unsubstituted C2 to C30 heterocyclic group, or a combination thereof.

5. The organic optoelectronic device as claimed in claim 1, wherein R¹ to R⁸ of Chemical Formula 1 are independently hydrogen, a substituted or unsubstituted phenyl group, a substituted or unsubstituted biphenyl group, a substituted or unsubstituted naphthyl group, a substituted or unsubstituted p-terphenyl group, a substituted or unsubstituted m-terphenyl group, a substituted or unsubstituted o-terphenyl group, a substituted or unsubstituted anthracenyl group, a substituted or unsubstituted phenanthrenyl group, a substituted or unsubstituted triphenylenyl group, or a substituted or unsubstituted fluorenyl group.

6. The organic optoelectronic device as claimed in claim 1, wherein the first compound for an organic optoelectronic device is selected from compounds of Group 1:

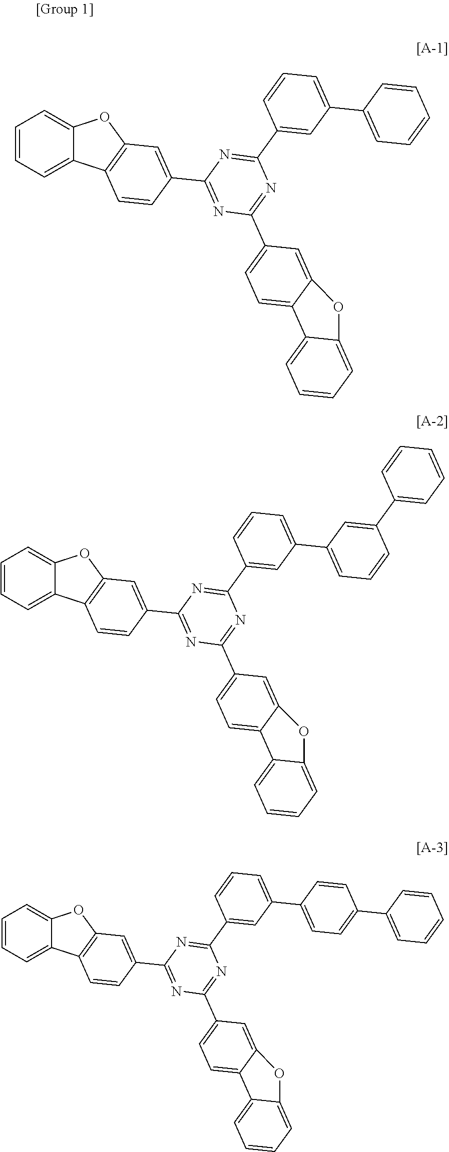 [SMILES: c1ccc(-c2cccc(-c3nc(-c4ccc5c(c4)oc4ccccc45)nc(-c4ccc5c(c4)oc4ccccc45)n3)c2)cc1 ; c1ccc(-c2cccc(-c3cccc(-c4nc(-c5ccc6c(c5)oc5ccccc56)nc(-c5ccc6c(c5)oc5ccccc56)n4)c3)c2)cc1 ; c1ccc(-c2ccc(-c3cccc(-c4nc(-c5ccc6c(c5)oc5ccccc56)nc(-c5ccc6c(c5)oc5ccccc56)n4)c3)cc2)cc1]

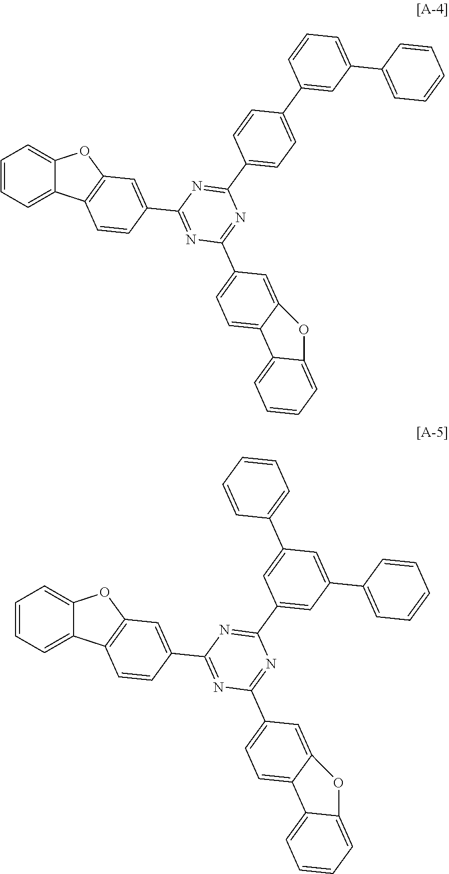 [SMILES: c1ccc(-c2cccc(-c3ccc(-c4nc(-c5ccc6c(c5)oc5ccccc56)nc(-c5ccc6c(c5)oc5ccccc56)n4)cc3)c2)cc1 ; c1ccc(-c2cc(-c3ccccc3)cc(-c3nc(-c4ccc5c(c4)oc4ccccc45)nc(-c4ccc5c(c4)oc4ccccc45)n3)c2)cc1]

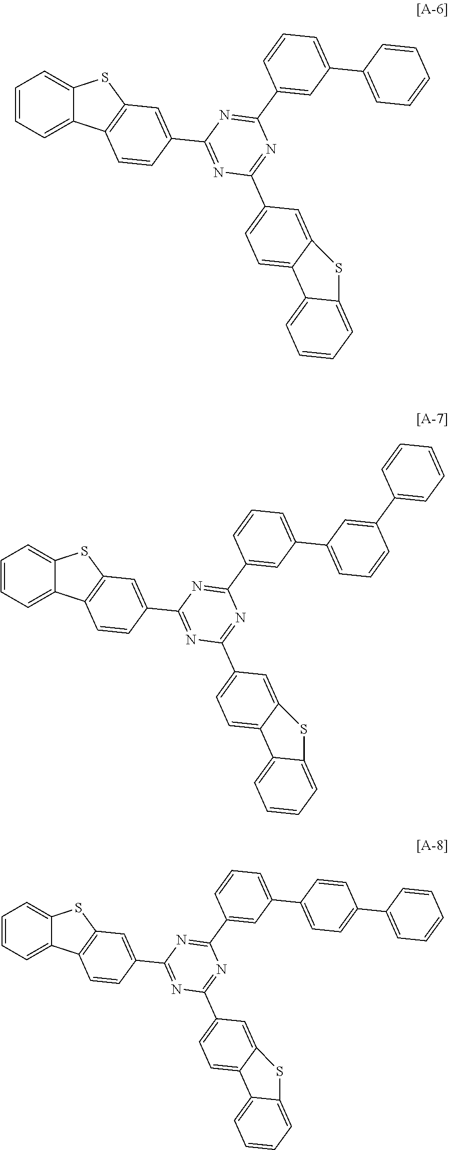 [SMILES: c1ccc(-c2cccc(-c3nc(-c4ccc5c(c4)sc4ccccc45)nc(-c4ccc5c(c4)sc4ccccc45)n3)c2)cc1 ; c1ccc(-c2cccc(-c3cccc(-c4nc(-c5ccc6c(c5)sc5ccccc56)nc(-c5ccc6c(c5)sc5ccccc56)n4)c3)c2)cc1 ; c1ccc(-c2ccc(-c3cccc(-c4nc(-c5ccc6c(c5)sc5ccccc56)nc(-c5ccc6c(c5)sc5ccccc56)n4)c3)cc2)cc1]

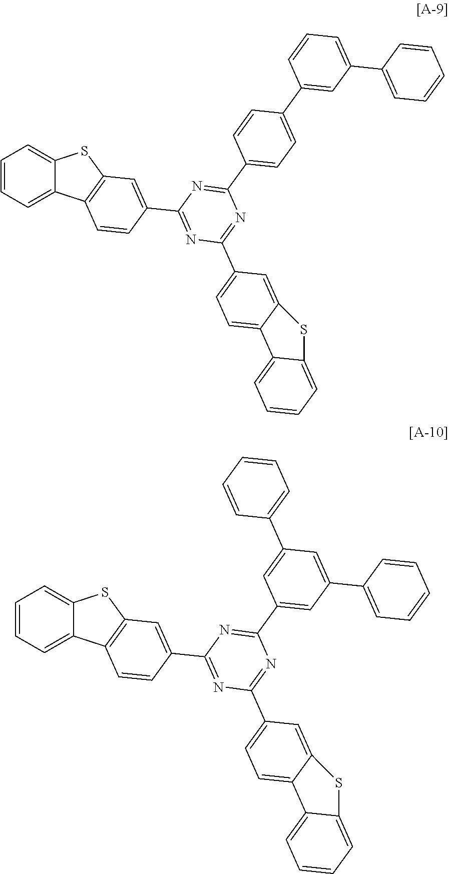 [SMILES: c1ccc(-c2cccc(-c3ccc(-c4nc(-c5ccc6c(c5)sc5ccccc56)nc(-c5ccc6c(c5)sc5ccccc56)n4)cc3)c2)cc1 ; c1ccc(-c2cc(-c3ccccc3)cc(-c3nc(-c4ccc5c(c4)sc4ccccc45)nc(-c4ccc5c(c4)sc4ccccc45)n3)c2)cc1]

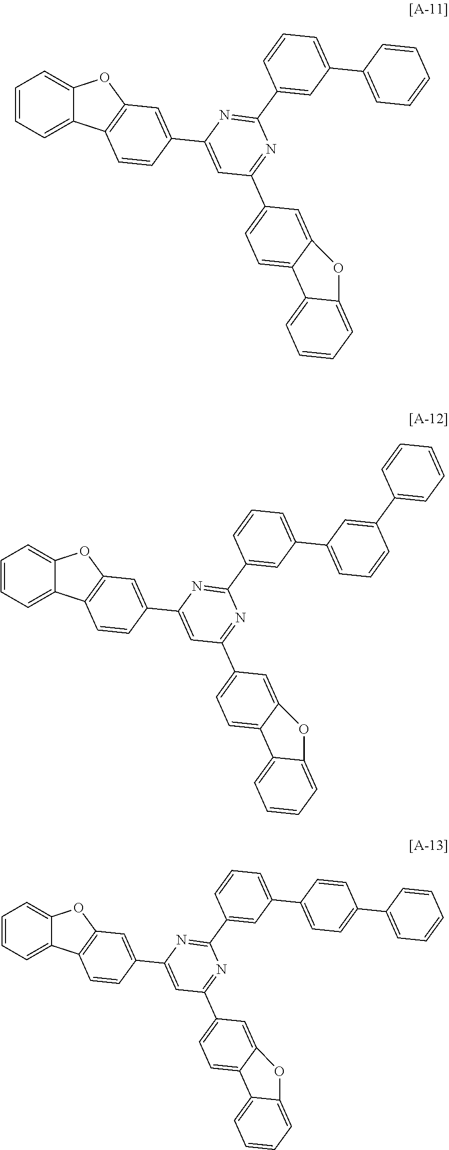 [SMILES: c1ccc(-c2cccc(-c3nc(-c4ccc5c(c4)oc4ccccc45)cc(-c4ccc5c(c4)oc4ccccc45)n3)c2)cc1 ; c1ccc(-c2cccc(-c3cccc(-c4nc(-c5ccc6c(c5)oc5ccccc56)cc(-c5ccc6c(c5)oc5ccccc56)n4)c3)c2)cc1 ; c1ccc(-c2ccc(-c3cccc(-c4nc(-c5ccc6c(c5)oc5ccccc56)cc(-c5ccc6c(c5)oc5ccccc56)n4)c3)cc2)cc1]

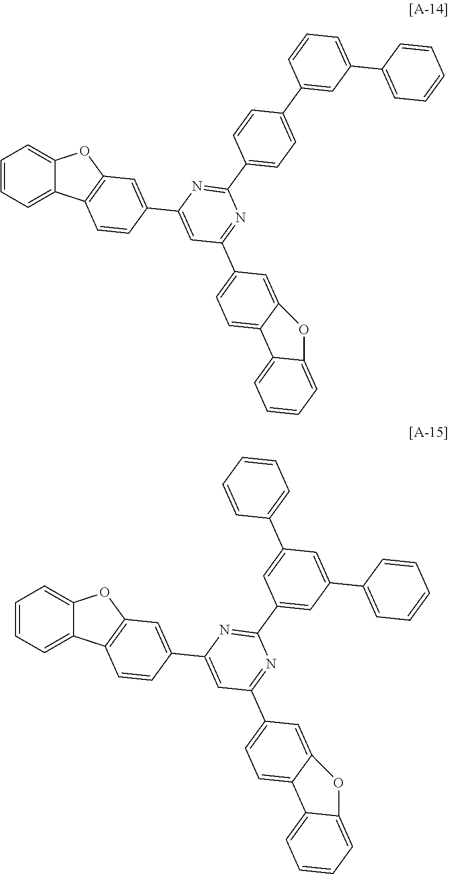 [SMILES: c1ccc(-c2cccc(-c3ccc(-c4nc(-c5ccc6c(c5)oc5ccccc56)cc(-c5ccc6c(c5)oc5ccccc56)n4)cc3)c2)cc1 ; c1ccc(-c2cc(-c3ccccc3)cc(-c3nc(-c4ccc5c(c4)oc4ccccc45)cc(-c4ccc5c(c4)oc4ccccc45)n3)c2)cc1]

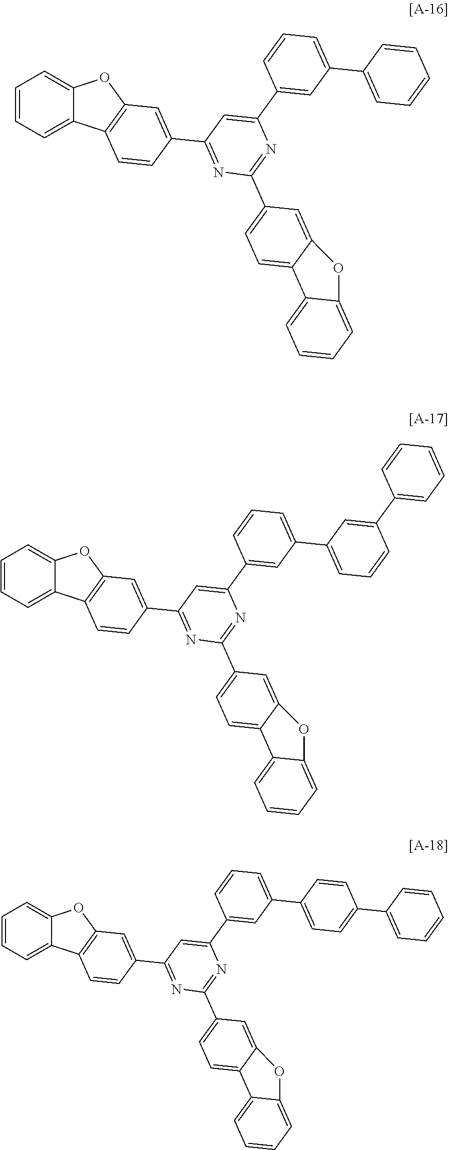 [SMILES: c1ccc(-c2cccc(-c3cc(-c4ccc5c(c4)oc4ccccc45)nc(-c4ccc5c(c4)oc4ccccc45)n3)c2)cc1 ; c1ccc(-c2cccc(-c3cccc(-c4cc(-c5ccc6c(c5)oc5ccccc56)nc(-c5ccc6c(c5)oc5ccccc56)n4)c3)c2)cc1 ; c1ccc(-c2ccc(-c3cccc(-c4cc(-c5ccc6c(c5)oc5ccccc56)nc(-c5ccc6c(c5)oc5ccccc56)n4)c3)cc2)cc1]

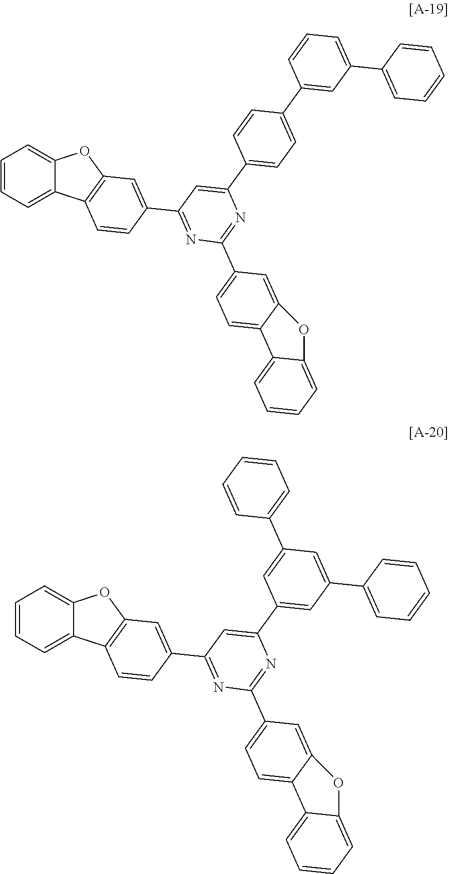 [SMILES: c1ccc(-c2cccc(-c3ccc(-c4cc(-c5ccc6c(c5)oc5ccccc56)nc(-c5ccc6c(c5)oc5ccccc56)n4)cc3)c2)cc1 ; c1ccc(-c2cc(-c3ccccc3)cc(-c3cc(-c4ccc5c(c4)oc4ccccc45)nc(-c4ccc5c(c4)oc4ccccc45)n3)c2)cc1]

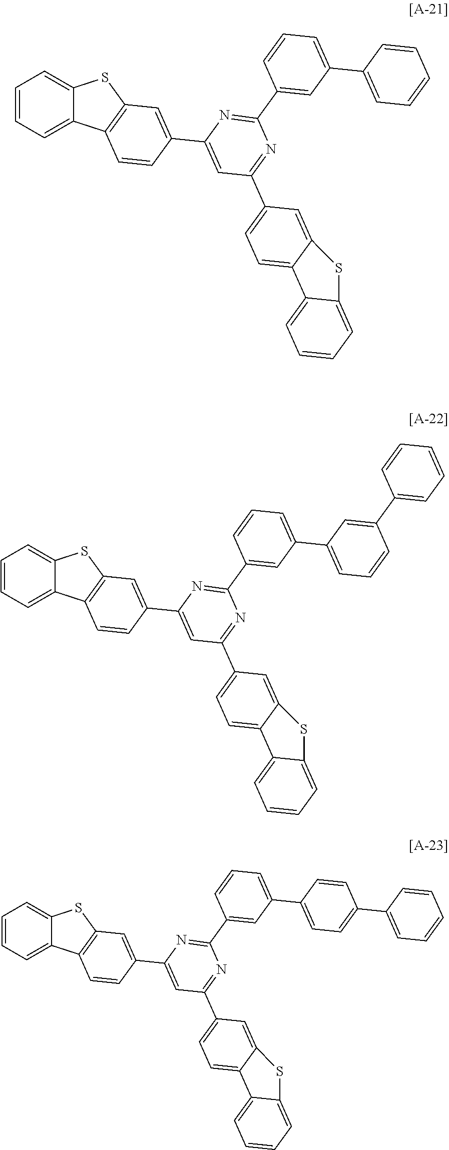 [SMILES: c1ccc(-c2cccc(-c3nc(-c4ccc5c(c4)sc4ccccc45)cc(-c4ccc5c(c4)sc4ccccc45)n3)c2)cc1 ; c1ccc(-c2cccc(-c3cccc(-c4nc(-c5ccc6c(c5)sc5ccccc56)cc(-c5ccc6c(c5)sc5ccccc56)n4)c3)c2)cc1 ; c1ccc(-c2ccc(-c3cccc(-c4nc(-c5ccc6c(c5)sc5ccccc56)cc(-c5ccc6c(c5)sc5ccccc56)n4)c3)cc2)cc1]

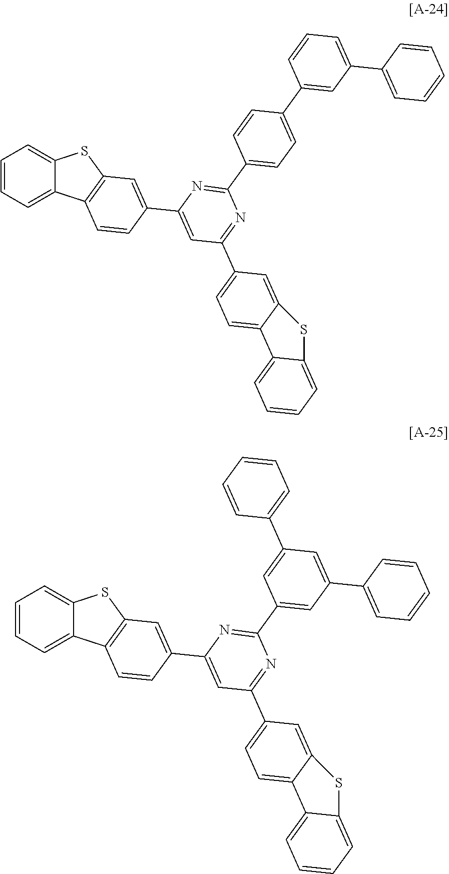 [SMILES: c1ccc(-c2cccc(-c3ccc(-c4nc(-c5ccc6c(c5)sc5ccccc56)cc(-c5ccc6c(c5)sc5ccccc56)n4)cc3)c2)cc1 ; c1ccc(-c2cc(-c3ccccc3)cc(-c3nc(-c4ccc5c(c4)sc4ccccc45)cc(-c4ccc5c(c4)sc4ccccc45)n3)c2)cc1]

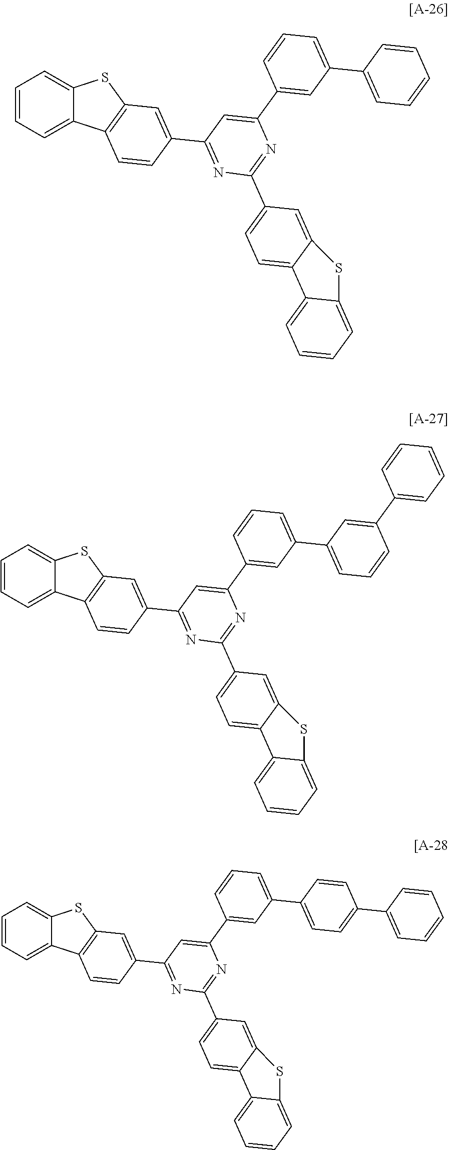 [SMILES: c1ccc(-c2cccc(-c3cc(-c4ccc5c(c4)sc4ccccc45)nc(-c4ccc5c(c4)sc4ccccc45)n3)c2)cc1 ; c1ccc(-c2cccc(-c3cccc(-c4cc(-c5ccc6c(c5)sc5ccccc56)nc(-c5ccc6c(c5)sc5ccccc56)n4)c3)c2)cc1 ; c1ccc(-c2ccc(-c3cccc(-c4cc(-c5ccc6c(c5)sc5ccccc56)nc(-c5ccc6c(c5)sc5ccccc56)n4)c3)cc2)cc1]

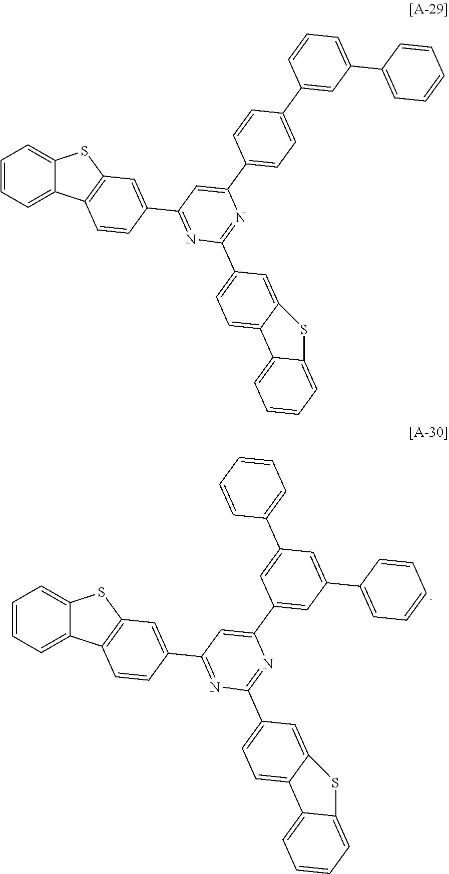 [SMILES: c1ccc(-c2cccc(-c3ccc(-c4cc(-c5ccc6c(c5)sc5ccccc56)nc(-c5ccc6c(c5)sc5ccccc56)n4)cc3)c2)cc1 ; c1ccc(-c2cc(-c3ccccc3)cc(-c3cc(-c4ccc5c(c4)sc4ccccc45)nc(-c4ccc5c(c4)sc4ccccc45)n3)c2)cc1]

7. The organic optoelectronic device as claimed in claim 1, wherein Ar¹ and Ar² of Chemical Formula 2 are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted dibenzothiophenyl group, a substituted or unsubstituted dibenzofuranyl group, a substituted or unsubstituted carbazolyl group, a substituted or unsubstituted fluorenyl group, or a combination thereof.

8. The organic optoelectronic device as claimed in claim 1, wherein Chemical Formula 2 is one of structures of Group I, and
*-L¹-Ar¹ and *-L²-Ar² are one of substituents of Group II: 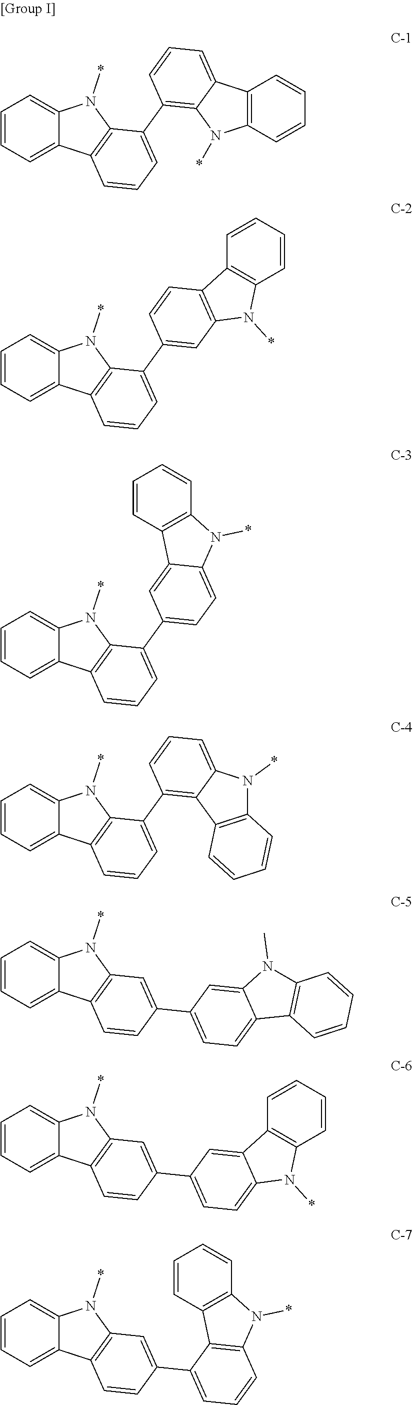 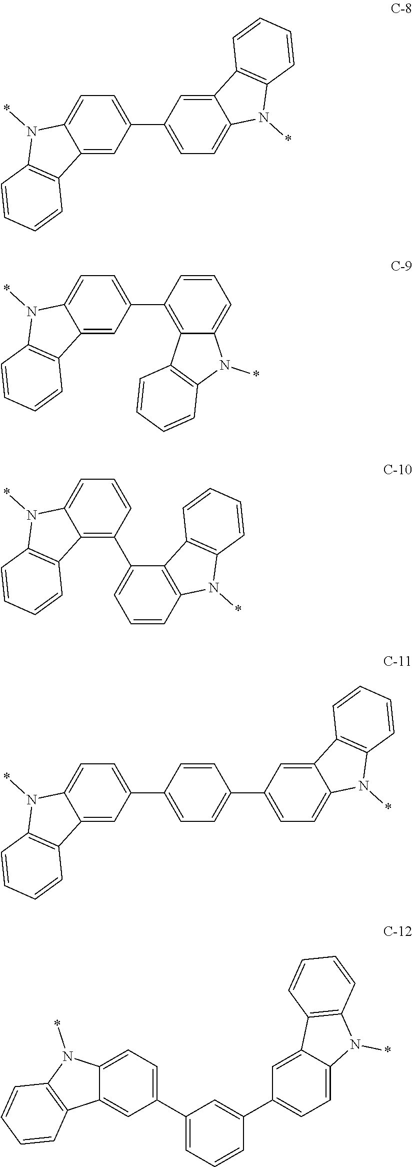 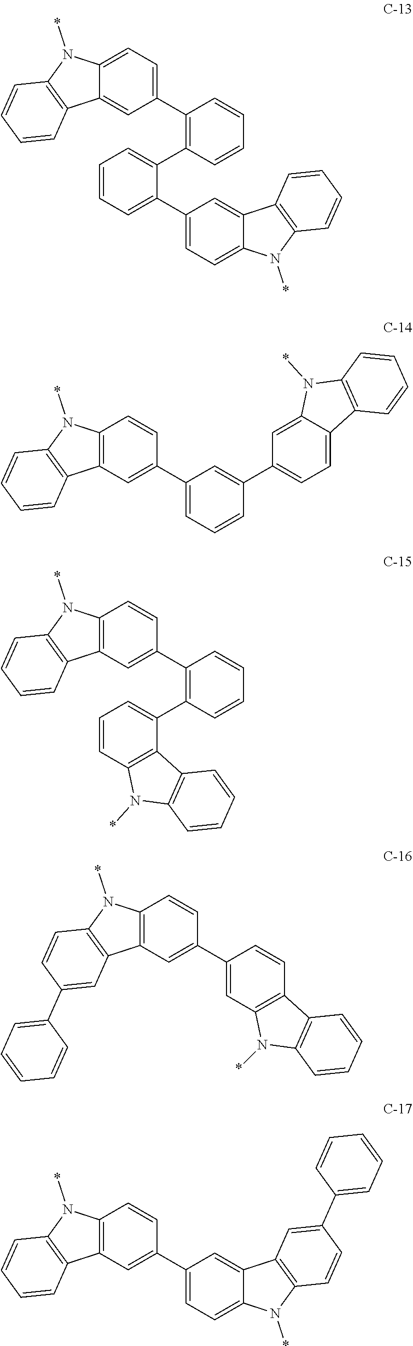 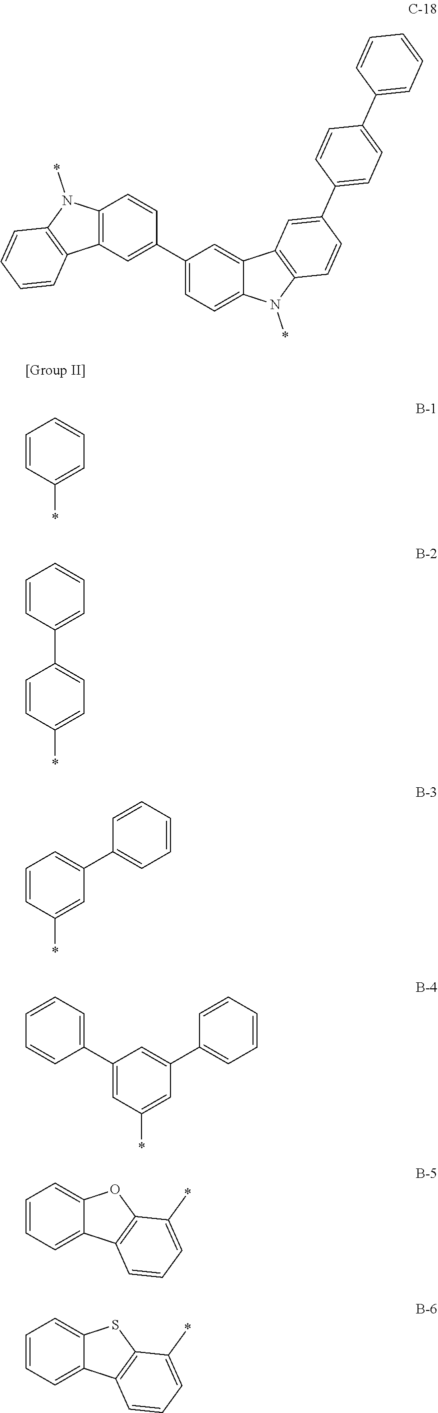 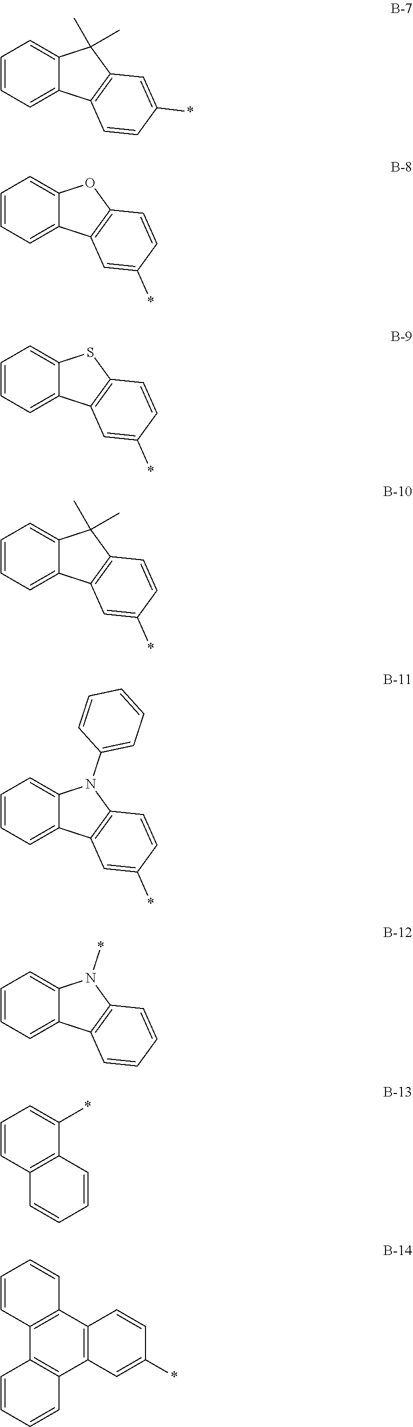 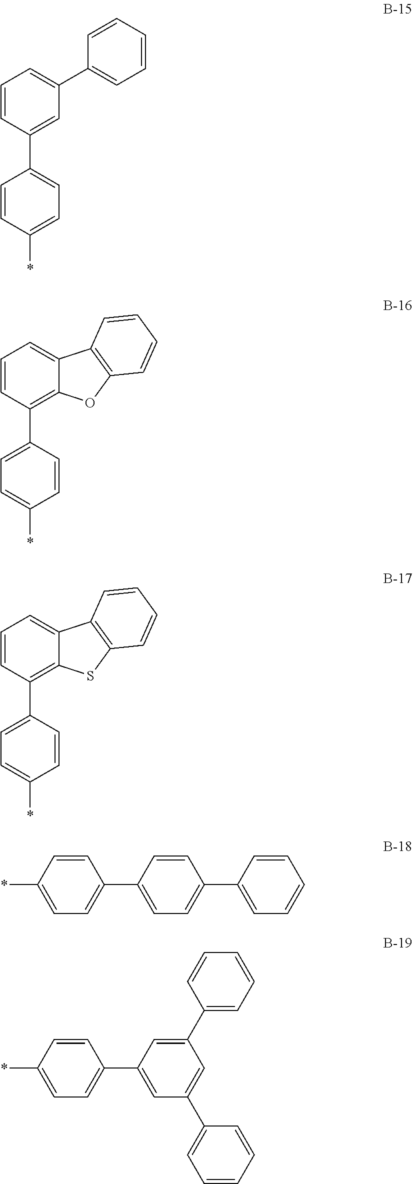
wherein, in Groups I and II, * is a linking point.

9. The organic optoelectronic device as claimed in claim 7, wherein Chemical Formula 2 is represented by Chemical Formula C-8 or Chemical Formula C-17 of Group I,
*-L¹-Ar¹ and *-L²-Ar² are selected from B-1, B-2, B-3, and B-16 of Group II.

10. The organic optoelectronic device as claimed in claim 1, wherein when A¹ to A³ of Chemical Formula 3 are independently present, at least one of A¹ to A³ is a substituted or unsubstituted fused aryl group or a substituted or unsubstituted fused heterocyclic group, and the substituted or unsubstituted fused aryl group or the substituted or unsubstituted fused heterocyclic group is selected from substituents of Group III: 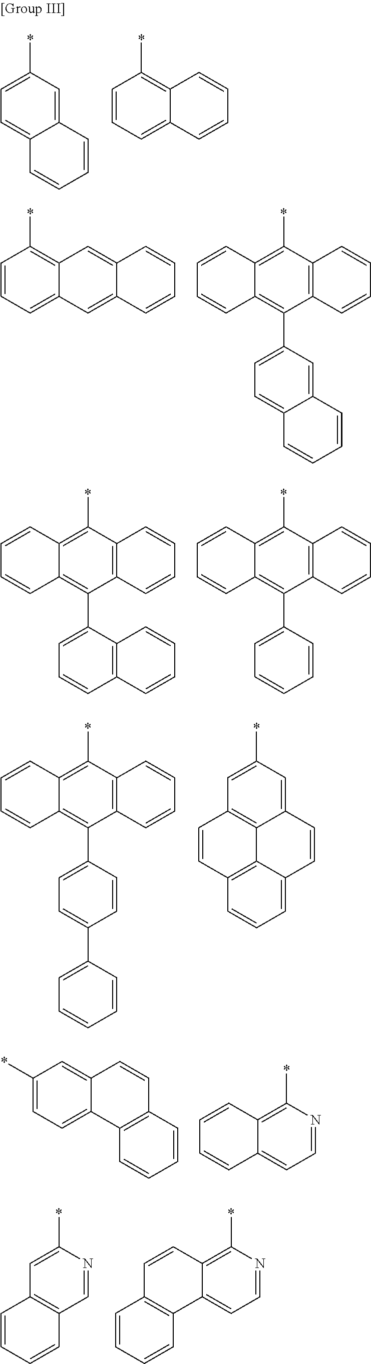 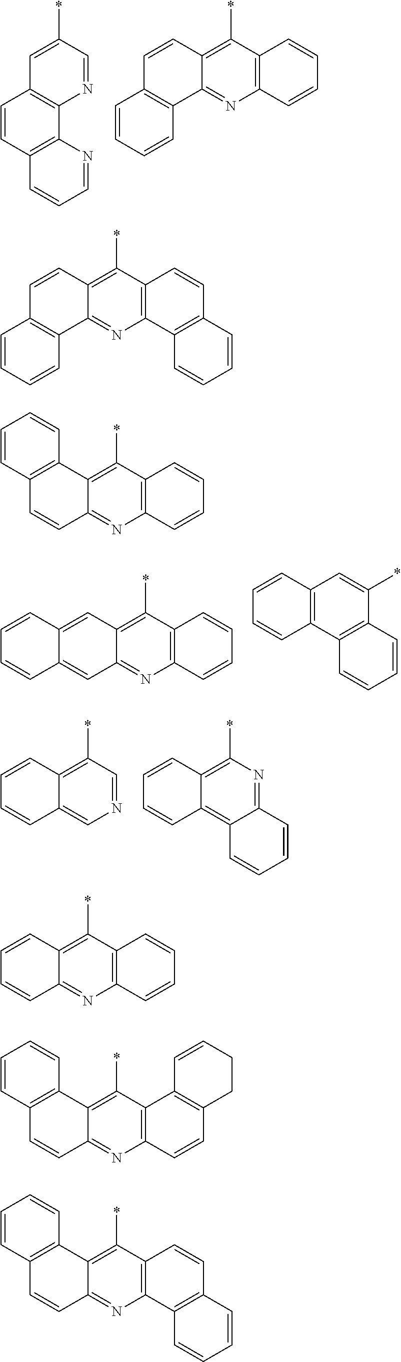
wherein, in Group III, * is a linking point.

11. The organic optoelectronic device as claimed in claim 1, wherein adjacent groups of A¹ to A³ are linked with each other and Chemical Formula 3 is represented by Chemical Formula 3a: 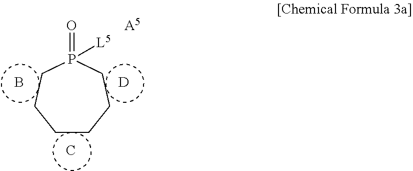
wherein, in Chemical Formula 3a,
B, C, and D are independently a substituted or unsubstituted C6 to C30 aryl or a substituted or unsubstituted C2 to C30 heterocyclic while sharing a septangular core and two carbons,
A³ is a substituted or unsubstituted C6 to C30 aryl group, a substituted or unsubstituted C2 to C30 heterocyclic group, or a combination thereof, and
L⁵ is a single bond, a substituted or unsubstituted C6 to C30 arylene group, a substituted or unsubstituted C2 to C30 heteroarylene group, or a combination thereof.

12. The organic optoelectronic device as claimed in claim 11, wherein B, C, and D are independently selected from substituted or unsubstituted moieties of Group IV: 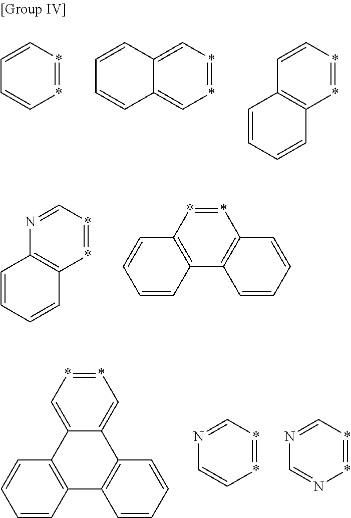
wherein, in Group IV, *'s indicate carbon shared with the septangular core of Chemical Formula 3a.

13. The organic optoelectronic device as claimed in claim 1, wherein the electron transport layer further includes a dopant.

14. The organic optoelectronic device as claimed in claim 1, which further includes a hole auxiliary layer between the anode and the light emitting layer.

15. A display device comprising the organic optoelectronic device as claimed in claim 1.
